(19) United States
(12) Patent Application Publication
(10) Pub. No.: US 2022/0168408 A1
(43) Pub. Date: Jun. 2, 2022

(54) MULTI-VALENT IMMUNOTHERAPY COMPOSITION AND METHODS OF USE FOR TREATING WT1-POSITIVE CANCERS

(71) Applicants: SLSG LIMITED LLC, DOVER, DE (US); MEMORIAL SLOAN KETTERING CANCER CENTER, NEW YORK, NY (US)

(72) Inventors: ANGELOS M. STERGIOU, NEW YORK, NY (US); NICHOLAS J. SARLIS, NEW YORK, NY (US); DAVID A. SCHEINBERG, NEW YORK, NY (US); TAO DAO, NEW YORK, NY (US)

(21) Appl. No.: 17/594,271

(22) PCT Filed: Apr. 10, 2020
(86) PCT No.: PCT/US2020/027681

§ 371 Date: Oct. 8, 2021

Related U.S. Application Data
(60) Provisional application No. 62/832,244, filed on Apr. 10, 2019.

(51) Int. Cl. A61K 39/00; A61K 39/395; A61P 35/00

(52) U.S. Cl. CPC A61K 39/001153; A61K 39/3955; A61P 35/00; A61K 2039/53; A61K 2039/5154; A61K 2039/5158; A61K 2039/572; A61K 2039/55566; A61K 2039/55505; A61K 2039/55544; A61K 2039/55516; A61K 2039/55522; A61K 2039/55527; A61K 2039/55577

(57) ABSTRACT

This invention provides methods of treating, reducing the incidence of, and inducing immune responses to a WT1-expressing cancer, by administering a combination of WT1 peptides including each of: YMFPNAPYL, RSDELVRHHNMHQRNMTKL, PGCNKRYFKLSHLQMHSRKHTG, SGQAYMFPNAPYLPSCLES, NLMNLGATL, WNLMNLGATLKGVAA, and WNYMNLGATLKGVAA, or cytotoxic T cells induced by the combination of WT1 peptides. The combination of WT1 peptides may be administered to the subject via a WT1 delivery agent, i.e., in peptide form, or in the form of nucleic acids encoding the WT1 peptides, or in the form of immune cells comprising nucleic acids encoding the WT1 peptides, and/or comprising or presenting the WT1 peptides. The WT1 delivery agents or CTLs can be administered to the subject in a single composition (as a heptavalent immunotherapy composition), or multiple compositions, resulting in delivery of all seven WT1 peptides and induction of an immune response against the WT1-expressing cancer.

### CROSS-REFERENCE TO RELATED APPLICATION

The present application claims the benefit of U.S. Provisional Application Ser. No. 62/832,244, filed Apr. 10, 2019, which is hereby incorporated by reference herein in its entirety, including any figures, tables, nucleic acid sequences, amino acid sequences, or drawings.

The Sequence Listing for this application is labeled “Seq-List.txt” which was created on Apr. 9, 2020 and is 47 KB. The entire content of the sequence listing is incorporated herein by reference in its entirety.

### BACKGROUND OF THE INVENTION

This invention provides methods of treating, reducing the incidence of, and inducing immune responses against a WT1-expressing cancer, and compositions useful for the same purposes.

### BRIEF SUMMARY OF THE INVENTION

This invention provides methods of treating, reducing the incidence of, and inducing immune responses against a WT1-expressing cancer, and compositions including immunogenic compositions useful for the same purposes. In one embodiment, the present invention provides methods for such use comprising administering to a subject in need thereof a combination of at least the following seven WT1 peptides: YMFPNAPYL (SEQ ID NO: 124), RSDELVRHHNMHQRNMTKL (SEQ ID NO: 1), PGCNKRYFKLSHLQMHSRKHTG (SEQ ID NO: 2), SGQAYMFPNAPYLPSCLES (SEQ ID NO: 125), NLMNLGATL (SEQ ID NO: 21), WNLMNLGATLKGVAA (SEQ ID NO: 26), and WNYMNLGATLKGVAA (SEQ ID NO: 205). In some embodiments, all seven peptides are present in a composition that is administered to the subject, as a multi-valent (heptavalent) immunotherapy composition.

The combination of at least seven WT1 peptides can be administered to the subject by administering one or more WT1 delivery agents to the subject resulting in delivery of the combination of WT1 peptides and induction of an immune response against the WT1-expressing cancer. Examples of these WT1 delivery agents that may be used include: (i) one or more of the isolated WT1 peptides themselves, (ii) one or more nucleic acids encoding the WT1 peptides, and (iii) one or more immune cells comprising or presenting the combination of WT1 peptides or nucleic acid encoding the combination of WT1 peptides, or any combination of two or three of (i), (ii), or (iii).

It may be desired to administer one or more of the at least seven WT1 peptides as peptides, and administer other of the at least seven WT1 peptides via other forms of WT1 deliver agent, such as nucleic acids encoding the WT1 peptides, or via immune cells comprising or presenting the WT1 peptides. Therefore, the at least seven WT1 peptides may be administered in the form of peptides, or in the form of nucleic acids encoding the peptides, or in the form of immune cells comprising or presenting the WT1 peptides or encoding nucleic acids, or in two or all three of these forms (i.e., peptides and nucleic acids; peptides and immune cells; nucleic acids and immune cells; or peptides, nucleic acids, and immune cells). Thus, the combination of at least seven WT1 peptides may be administered: (i) as seven or more peptides, individually or in any one or more combinations; or (ii) as nucleic acids encoding the combination of at least seven WT1 peptides, individually or in any one or more combinations; or (iii) immune cells comprising or presenting the combination of at least seven WT1 peptides or nucleic acids encoding the combination of at least seven or more peptides, individually or in any one or more combinations. In some embodiments, the at least seven WT1 peptides is administered as a combination of two or all three of these forms (i.e., (i) and (ii); or (i) and (iii); or (ii) and (iii); or (i), (ii), and (iii)).

Optionally, alternatively or in addition to the WT1 delivery agents (peptides, nucleic acids encoding the peptides, and immune cells), cytotoxic T cells (CTLs) against the WT1-expressing cancer may be administered to the subject, wherein the CTLs have been induced by the combination of the at least seven isolated peptides: YMFPNAPYL (SEQ ID NO: 124), RSDELVRHHNMHQRNMTKL (SEQ ID NO: 1), PGCNKRYFKLSHLQMHSRKHTG (SEQ ID NO: 2), SGQAYMFPNAPYLPSCLES (SEQ ID NO: 125), NLMNLGATL (SEQ ID NO: 21), WNLMNLGATLKGVAA (SEQ ID NO: 26), and WNYMNLGATLKGVAA (SEQ ID NO: 205).

Optionally, in addition to the combination of seven WT1 peptides, nucleic acids encoding the seven WT1 peptides, immune cells comprising or presenting the seven WT1 peptides and/or comprising nucleic acids encoding the WT1 peptides, or CTLs induced by the seven WT1 peptides, one or more additional WT1 peptides, or CTLs induced by the one or more additional WT1 peptides may be included in the composition or otherwise administered to the subject. The one or more additional WT1 peptides may be native peptides which are fragments of the WT1 protein, or they may be such peptides with one or more modifications that may enhance the immunogenicity thereof, or a mixture. Such modifications may be amino acid changes (e.g., heteroclitic peptides), or any other modification. The one or more additional WT1 peptides may be administered to the subject via a WT1 delivery agent, or CTLs against the WT1-expressing cancer and that have been induced by the one or more additional WT1 peptides may be administered.

Optionally, one or more checkpoint inhibitors may be administered to the subject before, during, or after administration of the WT1 delivery agents and/or CTLs. The one or more checkpoint inhibitors (also known as an immune checkpoint inhibitor) is a compound or agent that blocks or inhibits immune checkpoint proteins. Non-limiting examples of compounds or agents that are checkpoint inhibitors include small molecules, peptides, and antibodies. Non-limiting examples of antibodies include nivolumab (OPDIVO), pembrolizumab (KEYTRUDA), pidilizumab (CT-011), MEDI0680 (AMP-514), AMP-224, AUNP-12, BMS 936559, atezolizumab (MPDL3280A), durvalumab (MEDI4736), avelumab (MSB0010718C), BMS935559 (MDX-1105), rHIgM12B7, BMS-986016, GSK2831781, IMP321, lirilumab (BMS-986015), IPH2101 (1-7F9), Indoximod (NLG 9189), NLG 919, INCB024360, PF-05082566, Urelumab (BMS-663513), and MEDI6469.

In one embodiment, methods are embodied in which the one or more WT1 delivery agents or CTLs, and the one or more checkpoint inhibitor, are each administered to a subject according to a schedule that maximally benefits the subject. The one or more WT1 delivery agents or CTLs and the one or more checkpoint inhibitors are therefore not necessarily administered at the same time, or even in the same composition, or each for the same duration, or each by the same route. Each WT1 peptide may be administered in accordance with a particular schedule, as may be each checkpoint inhibitor. In one embodiment, the dosing schedules of the at least one WT1 peptide and the at least one checkpoint inhibitor are concurrent. In one embodiment, the dosing schedules of the at least one WT1 peptide and the at least one checkpoint inhibitor overlap. In one embodiment, at least one WT1 delivery agent or CTL and at least one checkpoint inhibitor are present in the same composition. In one embodiment, the methods embodied herein provide an enhanced or increased ability for treating, reducing the incidence of, and inducing immune responses against a WT1-expressing cancer, than the WT1 delivery agent(s) or CTLs and checkpoint inhibitor(s) alone. In one embodiment, the ability for treating, reducing the incidence of, and inducing immune responses against a WT1-expressing cancer provided by the methods described herein are greater than combination of the effect of the WT1 delivery agent(s) or CTLs alone and the checkpoint inhibitor(s) alone.

The dose level and dosing schedule of the WT1 delivery agent, or CTLs, and that of the checkpoint inhibitor, the route of administration, and other aspects of administration are optimized for maximal benefit to the subject. The embodiments herein provide improved methods of treating, reducing the incidence of, and inducing immune responses against a WT1-expressing cancer, and improved compositions useful for the same purposes.

Cancers amenable to the methods embodied herein are any cancers that express the WT1 protein or a fragment thereof. In one embodiment, the cancer is ovarian cancer. In another embodiment, the cancer is breast cancer. In another embodiment, the cancer is colon cancer or colorectal cancer. In another embodiment, the cancer is mesothelioma. In another embodiment, the cancer is leukemia. In other embodiments, the cancer is Wilms' tumor, acute myeloid leukemia (AML), multiple myeloma, chronic myeloid leukemia (CIVIL), myelodysplastic syndrome (MDS), melanoma, mesothelioma (e.g., malignant pleural mesothelioma), stomach cancer, prostate cancer, biliary cancer, urinary system cancer, glioblastoma, soft tissue sarcoma, osteosarcoma, or non-small cell lung cancer (NSCLC).

### DETAILED DESCRIPTION OF THE INVENTION

This invention provides methods of treating, reducing the incidence of, and inducing immune responses against a WT1-expressing cancer, and compositions including immunogenic compositions useful for the same purposes. In one embodiment, the present invention provides methods for such use comprising administering to a subject in need thereof a combination of at least seven WT1 peptides or cytotoxic T cells (CTLs) thereto, wherein the combination includes each of YMFPNAPYL (SEQ ID NO: 124; also referred to as WT1-A1), RSDELVRHHNMHQRNMTKL (SEQ ID NO: 1; also referred to as WT1-427 long), PGCNKRYFKLSHLQMHSRKHTG (SEQ ID NO: 2; also referred to as WT1-331 long), SGQAYMFPNAPYLPSCLES (SEQ ID NO: 125; also referred to as WT1-122A1 long), NLMNLGATL (SEQ ID NO: 21; also referred to as NLM short), WNLMNLGATLKGVAA (SEQ ID NO: 26; also referred to as WNLM or NLM long), and WNYMNLGATLKGVAA (SEQ ID NO: 205; also referred to as WNYM or NYM long). The combination of seven WT1 peptides may be administered with or without one or more checkpoint inhibitors. In some embodiments, the immunotherapy composition used for treating a WT1-expressing tumor or inducing in vitro, ex vivo or in vivo the formation and proliferation of T cells specific for a WT1-expressing cancer, and wherein the combination has a synergistic effect on one or more of the foregoing.

The combination of at least seven WT1 peptides can be administered to the subject by administering one or more agents to the subject resulting in delivery of the combination of at least seven WT1 peptides and induction of an immune response against the WT1-expressing cancer (i.e., one or more WT1 delivery agents). Each of the at least seven WT1 peptides may be delivered in one or more of the same or different WT1 delivery agents, and in one or more combinations thereof. Examples of these WT1 delivery agents that may be used include: (i) an isolated WT1 peptide, (ii) a nucleic acid encoding the at least one WT1 peptide, and (iii) an immune cell comprising or presenting the at least one WT1 peptide or nucleic acid encoding the at least one WT1 peptide. Thus, in some embodiments, the combination of at least seven WT1 peptides is administered in the form of seven isolated WT1 peptides. In some embodiments, a composition is administered to the subject, and the composition includes all seven isolated WT1 peptides.

Optionally, alternatively or in addition to the WT1 delivery agents (peptides, nucleic acids encoding the peptides, and immune cells), cytotoxic T cells (CTLs) against the WT1-expressing cancer may be administered to the subject, wherein the CTLs are induced by the combination of the at least seven isolated peptides: YMFPNAPYL (SEQ ID NO: 124), RSDELVRHHNMHQRNMTKL (SEQ ID NO: 1), PGCNKRYFKLSHLQMHSRKHTG (SEQ ID NO: 2), SGQAYMFPNAPYLPSCLES (SEQ ID NO: 125), NLMNLGATL (SEQ ID NO: 21), WNLMNLGATLKGVAA (SEQ ID NO: 26), and WNYMNLGATLKGVAA (SEQ ID NO: 205). CTLs include WT1-specific CTLs that are made in vitro or ex vivo or they may be made in vivo in a donor and obtained from the donor.

The WT1 delivery agents or CTLs may be provided in a composition with a carrier, excipient or diluent, among which may be an adjuvant. Non-limiting selections of the peptide component used in the methods and compositions embodied herein are described herein below.

Ovarian cancer is one of the most common gynecologic malignancies and the fifth most frequent cause of cancer death in women in the United States. Over 22,000 cases are diagnosed annually, and there are an estimated 15,500 deaths per year [1]. The majority of patients have widespread disease at presentation [2]. The 5-year survival for advanced-stage disease remains less than 30% [1]. Although a complete clinical remission following initial chemotherapy can be anticipated for many patients, a review of second-look laparotomy when it was often performed as a matter of routine care indicates that less than 50% of patients are actually free of disease [3]. Furthermore, nearly half of patients with a negative second look procedure relapse and require additional treatment [4]. Many patients will achieve a second complete clinical response with additional chemotherapy. However, almost all patients will relapse after a short remission interval of 9-11 months. [5]. Effective strategies to prolong remission or to prevent relapse are required, as subsequent remissions are of progressively shorter duration until chemotherapy resistance broadly develops [2].

Both antibody and T cell effectors have been shown to provide benefit in ovarian cancer models. Antibodies have been noted to curtail early tissue invasion [6]. Preclinical models have also demonstrated the clearance of circulating tumor cells and the elimination of systemic micro metastasis through the use of both passively administered and vaccine induced antibodies. With regards to T cell effectors, a globally activated immune response has been shown to be associated with improved clinical outcome in patients with advanced ovarian cancer. Zhang et al showed that the presence of tumor infiltrating T cells within tumor cell islets was associated with improvement in both progression free and overall survival [7]. Conversely, the infiltration of T-regulatory cells confers a worse prognosis [8].

Data in patients with ovarian cancer in second or greater remission confirms them to relapse in a predictable fashion [9]. In recent years, ovarian cancer has been targeted by a variety of novel immune based approaches. Antibody therapy has included oregovomab [10] which is a monoclonal antibody therapy targeting the CA125 antigen; abagovomab [11] which is an anti-idiotypic antibody targeting CA-125; and trastuzumab [12] which is a monoclonal humanized anti-HER2 antibody. Other strategies have included cytokine therapy such as Interferon-γ [13, 14] and IL-2 [15]. Active immunization with other antigens such as Lewis y [16], MUC1 [17], the HLA restricted peptide NY-ESO-1b [18] and the KH-1-KLH conjugate have also been evaluated. Previous strategies have been ineffective and new therapeutic modalities are needed to increase the efficacy of therapies for ovarian as well as numerous other cancers that are ineffectively treated with currently available therapies.

WT1 refers to Wilms' tumor 1 or the gene product of the WT1 gene. The Wilms' tumor suppressor gene, WT1, was first identified in childhood renal tumors, but WT1 is also highly expressed in multiple other hematologic malignancies and solid tumors including mesothelioma [19, 20]. WT1 was originally identified by cDNA mapping to a region of chromosome 11p13. The WT1 cDNA encodes a protein containing four Kruppel zinc fingers and contains a complex pattern of alternative splicing resulting in four different transcription factors. Each WT1 isoform has different DNA binding and transcriptional activities [21], and can positively or negatively regulate various genes involved in cellular proliferation, differentiation, apoptosis, organ development and sex determination. WT1 is normally expressed in tissues of the mesodermal origin during embryogenesis including the kidney, gonads, heart, mesothelium and spleen [22]. In normal adult tissues, WT1 expression is limited to low levels in the nuclei of normal CD34+ hematopoietic stem cells, myoepithelial progenitor cells, renal podocytes and some cells in the testis and ovary [23]. WT1 is highly homologous in mice and humans (96% at the amino acid level) and has similar tissue distribution and function [24, 25]. Although originally described as a tumor suppressor gene, the WT1 proteins appear to be involved in tumorigenesis.

The strong expression of WT1 protein in ovarian cancer coupled with its proposed mechanism of action make it a rational target for immunotherapy, among many other cancers that also express WT1 protein, such as but not limited to mesothelioma, leukemia, Wilms' tumor, acute myelogenous leukemia (AML), chronic myeloid leukemia (CML), myelodysplastic syndrome (MDS), melanoma, stomach cancer, prostate cancer, biliary cancer, urinary system cancer, glioblastoma, soft tissue sarcoma, osteosarcoma, and non-small cell lung cancer (NSCLC). In ovarian cancer, the expression is so frequent that pathologists routinely use immunohistochemical stains for WT1 (with a standardized convention for describing expression and determining as “positive” or “negative” to help distinguish epithelial ovarian cancers from other tumors. WT1 is a particularly sensitive and specific marker for serous ovarian cancer [26]. Ovarian tissue microarrays suggest that 70-80% of serous ovarian cancers express WT1 such that the majority of patients will have the target and be eligible for study participation.

One or more additional WT1 peptides that may be used in combination with the seven WT1 peptides may be native peptides which are fragments of the WT1 protein. In one embodiment, the additional one or more WT1 peptides is LVRHHNMHQRNMTKL (SEQ ID NO:3) or NKRYFKLSHLQMHSR (SEQ ID NO:4). In another embodiment the one or more additional peptides is SGQARMFPNAPYLPSCLES (SEQ ID NO:5) or QARMFPNAPYLPSCL (SEQ ID NO:6). In another embodiment, the additional one or more peptides is selected from among RMFPNAPYL (SEQ ID NO:7), SLGEQQYSV (SEQ ID NO:8), ALLPAVPSL (SEQ ID NO:9), NLGATLKGV (SEQ ID NO:10), DLNALLPAV (SEQ ID NO:11), GVFRGIQDV (SEQ ID NO:12), KRYFKLSHL (SEQ ID NO:13), ALLLRTPYS (SEQ ID NO:14), CMTWMQMNL (SEQ ID NO:15), NMHQRNMTK (SEQ ID NO:16), QMNLGATLK (SEQ ID NO:17), FMCAYPGCNK (SEQ ID NO:18), or KLSHLQMHSR (SEQ ID NO:19).

In another embodiment, the additional one or more WT1 peptides is selected from among NQMNLGATL (SEQ ID NO:20), NYMNLGATL (SEQ ID NO:22), CMTWNQMNLGATLKG (SEQ ID NO:23), CMTWNLMNLGATLKG (SEQ ID NO:24), WNQMNLGATLKGVAA (SEQ ID NO:25), MTWNQMNLGATLKGV (SEQ ID NO:27), TWNQMNLGATLKGVA (SEQ ID NO:28), CMTWNLMNLGATLKG (SEQ ID NO:29), MTWNLMNLGATLKGV (SEQ ID NO:30), TWNLMNLGATLKGVA (SEQ ID NO:31), WNLMNLGATLKGVAA (SEQ ID NO:32), MTWNYIVINLGATLKGV (SEQ ID NO:33), TWNYMNLGATLKGVA (SEQ ID NO:34), CMTWNQMNLGATLKGVA (SEQ ID NO:35), WNQMNLGAT (SEQ ID NO:36), TWNQMNLGA (SEQ ID NO:37), MTWNQMNLG (SEQ ID NO:38), CMTWNLMNLGATLKGVA (SEQ ID NO:39), WNLMNLGAT (SEQ ID NO:40), MNLGATLKG (SEQ ID NO:41), MTWNQMNLG (SEQ ID NO:42), CMTWNYMNLGATLKGVA (SEQ ID NO:43), MNLGATLKG (SEQ ID NO:44), MTWNQMNLG (SEQ ID NO:45), GALRNPTAC (SEQ ID NO:46), GYLRNPTAC (SEQ ID NO:47), GALRNPTAL (SEQ ID NO:48), YALRNPTAC (SEQ ID NO:49), GLLRNPTAC (SEQ ID NO:50), RQRPHPGAL (SEQ ID NO:51), RYRPHPGAL (SEQ ID NO:52), YQRPHPGAL (SEQ ID NO:53), RLRPHPGAL (SEQ ID NO:54), RIRPHPGAL (SEQ ID NO:55), GALRNPTAC (SEQ ID NO:56), GALRNPTAL (SEQ ID NO:57), RQRPHPGAL (SEQ ID NO:58), RLRPHPGAL (SEQ ID NO:59), RIRPHPGAL (SEQ ID NO:60), QFPNHSFKHEDPMGQ (SEQ ID NO:61), QFPNHSFKHEDPMGQ (SEQ ID NO:62), HSFKHEDPM (SEQ ID NO:63), HSFKHEDPY (SEQ ID NO:64), HSFKHEDPK (SEQ ID NO:65), KRPFMCAYPGCYKRY (SEQ ID NO:66), SEKRPFMCAYPGCNK (SEQ ID NO:67), KRPFMCAYPGCNK (SEQ ID NO:68), FMCAYPGCN (SEQ ID NO:69), FMCAYPGCY (SEQ ID NO:70), or FMCAYPGCK (SEQ ID NO:71).

In another embodiment, the one or more additional WT1 peptides is selected from among RQRPHPGAL (SEQ ID NO:72), GALRNPTAC (SEQ ID NO:73), PLPHFPPSL (SEQ ID NO:74), HFPPSLPPT (SEQ ID NO:75), THSPTHPPR (SEQ ID NO:76), AILDFLLLQ (SEQ ID NO:77), PGCLQQPEQ (SEQ ID NO:78), PGCLQQPEQQG (SEQ ID NO:79), KLGAAEASA (SEQ ID NO:80), ASGSEPQQM (SEQ ID NO:81), RDLNALLPAV (SEQ ID NO:82), GGCALPVSGA (SEQ ID NO:83), GAAQWAPVL (SEQ ID NO:84), LDFAPPGAS (SEQ ID NO:85), LDFAPPGASAY (SEQ ID NO:86), SAYGSLGGP (SEQ ID NO:87), PAPPPPPPP (SEQ ID NO:88), ACRYGPFGP (SEQ ID NO:89), SGQARMFPN (SEQ ID NO:90), RMFPNAPYL (SEQ ID NO:91), PSCLESQPA (SEQ ID NO:92), NQGYSTVTF (SEQ ID NO:93), HHAAQFPNH (SEQ ID NO:94), HSFKHEDPM (SEQ ID NO:95), CHTPTDSCT (SEQ ID NO:96), CTGSQALLL (SEQ ID NO:97), TDSCTGSQA (SEQ ID NO:98), RTPYSSDNL (SEQ ID NO:99), NLYQMTSQLE (SEQ ID NO:100), WNQMNLGAT (SEQ ID NO:101), NQMNLGATL (SEQ ID NO:102), WNQMNLGATLK (SEQ ID NO:103), CMTWNQMNLGATLKG (SEQ ID NO:104), NLGATLKGV (SEQ ID NO:105), LGATLKGVAA (SEQ ID NO:106), TLGVAAGS (SEQ ID NO:107), GYESDNHTT (SEQ ID NO:108), FMCAYPGCNK (SEQ ID NO:109), KRPFMCAYPGC (SEQ ID NO:110), RKFSRSDHL (SEQ ID NO:111), LKTHTTRTHT (SEQ ID NO:112), NMHQRNHTKL (SEQ ID NO:113), LLAAILDFL (SEQ ID NO:114), CLQQPEQQGV (SEQ ID NO:115), DLNALLPAV (SEQ ID NO:116), ALLPAVPSL (SEQ ID NO:117), VLDFAPPGA (SEQ ID NO:118), CMTWNQMNL (SEQ ID NO:119), QARMFPNAPY (SEQ ID NO:120), ALRNPTACPL (SEQ ID NO:121), YPGCNKRYF (SEQ ID NO:122) or APVLDFAPPGASAYG (SEQ ID NO:123).

In another embodiment, the one or more additional WT1 peptides is any peptide described in WO2017087857, WO2014113490, or WO2019006401. The foregoing are incorporated herein by reference in their entireties.

In another embodiment, the one or more additional WT1 peptides is any native WT1 peptide described in WO2005053618, WO2007047763, WO2007047764, WO2007120673, US20060084609, WO2014113490 and WO2013106834. The foregoing are incorporated herein by reference in their entireties.

In another embodiment, the one or more additional WT1 peptides is any native WT1 peptide described in US20110070251A1, U.S. Pat. Nos. 7,063,854B1, 7,063,854, 7,901,693, 7,662,386, 7,063,854, 7,115,272, 7,368,119, 7,329,410, 7,144,581, 7,323,181, 7,655,249, 7,553,494, 7,608,685, 7,380,871, 7,030,212, 7,807,792, 7,517,950, US2010/0166738, US2011/0070251, US2009/0143291 and WO2003037060. The foregoing are incorporated herein by reference in their entireties.

In another embodiment, the one or more additional WT1 peptides is any native WT1 peptide described in U.S. Pat. No. 7,666,985B2, US20080070835A1, US20070128207A1, U.S. Pat. No. 7,915,393B2, US20110136141A1, U.S. Pat. No. 7,598,221B2, US20100111986A1, US20100092522A1, US20030082194A1 and WO2001025273A2. The foregoing are incorporated herein by reference in their entireties.

The one or more additional WT1 peptides may be a modified WT1 peptide fragment, such as containing one or more heteroclitic modifications to enhance immunogenicity against the native peptide sequence. In another embodiment the additional WT1 peptide is QAYMFPNAPYLPSCL (SEQ ID NO:126). In another embodiment, the one or more additional peptides is any of among YLGEQQYSV (SEQ ID NO:127), YLLPAVPSL (SEQ ID NO:128), YLGATLKGV (SEQ ID NO:129), YLNALLPAV (SEQ ID NO:130), GLRRGIQDV (SEQ ID NO:131), KLYFKLSHL (SEQ ID NO:132), ALLLRTPYV (SEQ ID NO:133), YMTWNQMNL (SEQ ID NO:134), NMYQRNMTK (SEQ ID NO:135), NMHQRVMTK (SEQ ID NO:136), NMYQRVMTK (SEQID NO: 137), QMYLGATLK (SEQ ID NO:138), QMNLGVTLK (SEQ ID NO:139), QMYLGVTLK (SEQ ID NO: 140), FMYAYPGCNK (SEQ ID NO:141), FMCAYPFCNK (SEQ ID NO:142), FMYAYPFCNK (SEQ ID NO:143), KLYHLQMHSR (SEQ ID NO:144), KLSHLQMHSK (SEQ ID NO:145), and KLYHLQMHSK (SEQ ID NO:146).

In another embodiment, the one or more additional WT1 peptides is any modified WT1 peptide from among NQMNLGATL (SEQ ID NO:147), NYMNLGATL (SEQ ID NO:149), CMTWNQMNLGATLKG (SEQ ID NO:150), CMTWNLMNLGATLKG (SEQ ID NO:151), WNQMNLGATLKGVAA (SEQ ID NO:152), MTWNQMNLGATLKGV (SEQ ID NO:154), TWNQMNLGATLKGVA (SEQ ID NO:155), CMTWNLMNLGATLKG (SEQ ID NO:156), MTWNLMNLGATLKGV (SEQ ID NO:157), TWNLMNLGATLKGVA (SEQ ID NO:158), WNLMNLGATLKGVAA (SEQ ID NO:159), MTWNYMNLGATLKGV (SEQ ID NO:160), TWNYMNLGATLKGVA (SEQ ID NO:161), CMTWNQMNLGATLKGVA (SEQ ID NO:162), WNQMNLGAT (SEQ ID NO:163), TWNQMNLGA (SEQ ID NO:164), MTWNQMNLG (SEQ ID NO:165), CMTWNLMNLGATLKGVA (SEQ ID NO:166), WNLMNLGAT (SEQ ID NO:167), MNLGATLKG (SEQ ID NO:168), MTWNQMNLG (SEQ ID NO:169), CMTWNYMNLGATLKGVA (SEQ ID NO:170), MNLGATLKG (SEQ ID NO:171), MTWNQMNLG (SEQ ID NO:172), GALRNPTAC (SEQ ID NO:173), GYLRNPTAC (SEQ ID NO:174), GALRNPTAL (SEQ ID NO:175), YALRNPTAC (SEQ ID NO:176), GLLRNPTAC (SEQ ID NO:177), RQRPHPGAL (SEQ ID NO:178), RYRPHPGAL (SEQ ID NO:179), YQRPHPGAL (SEQ ID NO:180), RLRPHPGAL (SEQ ID NO:181), RIRPHPGAL (SEQ ID NO:182), GALRNPTAC (SEQ ID NO:183), GALRNPTAL (SEQ ID NO:184), RQRPHPGAL (SEQ ID NO:185), RLRPHPGAL (SEQ ID NO:186), RIRPHPGAL (SEQ ID NO:187), QFPNHSFKHEDPMGQ (SEQ ID NO:188), QFPNHSFKHEDPMGQ (SEQ ID NO:189), HSFKHEDPM (SEQ ID NO:190), HSFKHEDPY (SEQ ID NO:191), HSFKHEDPK (SEQ ID NO:192), KRPFMCAYPGCYKRY (SEQ ID NO:193), SEKRPFMCAYPGCNK (SEQ ID NO:194), KRPFMCAYPGCNK (SEQ ID NO:195), FMCAYPGCN (SEQ ID NO:196), FMCAYPGCY (SEQ ID NO:197), or FMCAYPGCK (SEQ ID NO:198).

In another embodiment, the WT1 peptide is any modified WT1 peptide described in WO2005053618, WO2007047763, WO2007047764, WO2007120673, US20060084609, WO2014113490 and WO2013106834. The foregoing are incorporated herein by reference in their entireties.

In another embodiment, the WT1 peptide is any modified WT1 peptide described in US20110070251A1, U.S. Pat. No. 7,063,854B1, U.S. Pat. Nos. 7,063,854, 7,901,693, 7,662,386, 7,063,854, 7,115,272, 7,368,119, 7,329,410, 7,144,581, 7,323,181, 7,655,249, 7,553,494, 7,608,685, 7,380,871, 7,030,212, 7,807,792, 7,517,950, US2010/0166738, US2011/0070251, US2009/0143291 and WO2003037060. The foregoing are incorporated herein by reference in their entireties.

In another embodiment, the WT1 peptide is any modified WT1 peptide described in U.S. Pat. No. 7,666,985B2, US20080070835A1, US20070128207A1, U.S. Pat. No. 7,915,393B2, US20110136141A1, U.S. Pat. No. 7,598,221B2, US20100111986A1, US20100092522A1, US20030082194A1 and WO2001025273A2. The foregoing are incorporated herein by reference in their entireties.

The one or more additional WT1 peptides useful for the purposes described herein may be a single peptide or a combination of peptides. Each of the one or more additional WT1 peptides may be a native WT1 peptide or a modified WT1 peptide. If two or more peptides are used, each may be administered individually (in separate formulations) or in a combination with another one or more peptides (in the same formulation). The one or more peptides may be administered in combination with a carrier, diluent or excipient. In one embodiment, the peptide is administered in combination with an adjuvant. Each peptide may be administered with a different adjuvant or combination of adjuvants, or peptides may be administered in a combination of two or more peptides, with an adjuvant of combination of adjuvants. The immunogen or composition containing the one or more peptides may be referred to herein as a vaccine, a peptide vaccine, a WT1 vaccine, and the like.

The adjuvant may be of any class such as alum salts and other mineral adjuvants, bacterial products or bacteria-derived adjuvants, tensoactive agents (e.g., saponins), oil-in-water (o/w) and water-in-oil (w/o) emulsions, liposome adjuvants, cytokines (e.g., IL-2, GM-CSF, IL-12, and IFN-gamma), and alpha-galactosylceramide analogs. Nonlimiting examples of adjuvants include Montanide emulsions, QS21, Freund's complete or incomplete adjuvant, aluminum phosphate, aluminum hydroxide, Bacillus Calmette-Guerin (BCG), and alum. In one embodiment, the adjuvant is an agent that enhances the immune system's CTL response against the WT1 peptide, such as the surfactant mannide monooleate containing vegetable-grade (VG) oleic acid derived from olive oil (Montanide ISA 51 VG w/o emulsion). The adjuvant may be administered in the same composition as the one or more WT1 peptides, or in the same composition as the one or more checkpoint inhibitors, or in the same composition as both the one or more WT1 peptides and the one or more checkpoint inhibitors, or in a composition separate from the one or more WT1 peptides and one or more checkpoint inhibitors. In another embodiment, any of the aforementioned peptides (the combination of seven WT1 peptides, and, optionally, one or more additional WT1 peptides, has one or more point mutations in a primary or secondary anchor residue of the HLA class I binding motif. In one embodiment, the peptide has a point mutation at position 2 or 9 of the class I binding motif, or in secondary anchor residue position 1, 3, 4, 5, 6, 7 or 8 of the class I binding motif. In one embodiment, the peptide, position 1 of the HLA class I binding motif is changed to glycine, threonine or phenylalanine; in one embodiment, position 2 of the HLA class I binding motif is changed to leucine or isoleucine; in one embodiment, position 6 of the HLA class I binding motif is changed to valine, glutamine or histidine; or in one embodiment, position 9 of the HLA class I binding motif is changed to valine, alanine, threonine, isoleucine, or cysteine.

Optionally, the combination of seven WT1 peptides further includes one or more native or modified WT1 peptides from among those disclosed in WO2014113490, such as NQMNLGATL (SEQ ID NO:147), NLMNLGATL (SEQ ID NYMNLGATL (SEQ ID NO:149), CMTWNQMNLGATLKG (SEQ ID NO:150), CMTWNLMNLGATLKG (SEQ ID NO:151), WNQMNLGATLKGVAA (SEQ ID NO:152), MTWNQMNLGATLKGV (SEQ ID NO:154), TWNQMNLGATLKGVA (SEQ ID NO:155), CMTWNLMNLGATLKG (SEQ ID NO:156), MTWNLMNLGATLKGV (SEQ ID NO:157), TWNLMNLGATLKGVA (SEQ ID NO:158), WNLMNLGATLKGVAA (SEQ ID NO:159), MTWNYMNLGATLKGV (SEQ ID NO:1260), TWNYMNLGATLKGVA (SEQ ID NO:161), CMTWNQMNLGATLKGVA (SEQ ID NO:162), WNQMNLGAT (SEQ ID NO:163), TWNQMNLGA (SEQ ID NO:164), MTWNQMNLG (SEQ ID NO:165), CMTWNLMNLGATLKGVA (SEQ ID NO:166), WNLMNLGAT (SEQ ID NO:167), MNLGATLKG (SEQ ID NO:168), MTWNQMNLG (SEQ ID NO:169), CMTWNYMNLGATLKGVA (SEQ ID NO:170), MNLGATLKG (SEQ ID NO:171), MTWNQMNLG (SEQ ID NO:172), GALRNPTAC (SEQ ID NO:173), GYLRNPTAC (SEQ ID NO:174), GALRNPTAL (SEQ ID NO:175), YALRNPTAC (SEQ ID NO:176), GLLRNPTAC (SEQ ID NO:177), RQRPHPGAL (SEQ ID NO:178), RYRPHPGAL (SEQ ID NO:179), YQRPHPGAL (SEQ ID NO:180), RLRPHPGAL (SEQ ID NO:181), RIRPHPGAL (SEQ ID NO:182), GALRNPTAC (SEQ ID NO:183), GALRNPTAL (SEQ ID NO:184), RQRPHPGAL (SEQ ID NO:185), RLRPHPGAL (SEQ ID NO:186), RIRPHPGAL (SEQ ID NO:187), QFPNHSFKHEDPMGQ (SEQ ID NO:188), QFPNHSFKHEDPMGQ (SEQ ID NO:189), HSFKHEDPM (SEQ ID NO:190), HSFKHEDPY (SEQ ID NO:191), HSFKHEDPK (SEQ ID NO:192), KRPFMCAYPGCYKRY (SEQ ID NO:194), SEKRPFMCAYPGCNK (SEQ ID NO:194), KRPFMCAYPGCNK (SEQ ID NO:195), FMCAYPGCN (SEQ ID NO:196), FMCAYPGCY (SEQ ID NO:197), or FMCAYPGCK (SEQ ID NO:198).

Each peptide of a combination may be administered separately within its own formulation, or two, three, four, five, six, or seven, or more peptides of a combination may be administered together within the same formulation. In one embodiment, the combination of at least seven WT1 peptides is administered within the same formulation.

The dose level of each peptide, the frequency of administration of each individual or any one or more combinations of up to seven or more peptides, the duration of administration and other aspects of the immunization with the seven or more WT1 peptides may be optimized in accordance with the patient's clinical presentation, duration or course of the disease, comorbidities, and other aspects of clinical care. The invention is not so limiting with regard to the particular aspects of the immunization component of the methods embodied herein.

In one embodiment, the multi-valent immunotherapy composition comprises 280 mcg of each of the seven aforementioned peptides: YMFPNAPYL (SEQ ID NO: 124), RSDELVRHHNMHQRNMTKL (SEQ ID NO: 1), PGCNKRYFKLSHLQMHSRKHTG (SEQ ID NO: 2), SGQAYMFPNAPYLPSCLES (SEQ ID NO: 125), NLMNLGATL (SEQ ID NO: 21), WNLMNLGATLKGVAA (SEQ ID NO: 26), and WNYMNLGATLKGVAA (SEQ ID NO: 205). In one embodiment, the composition further comprises one or more additional WT1 peptides. In one embodiment, the composition includes no further WT1 peptides. In one embodiment, the composition includes no further peptides.

In one embodiment, 200 mcg of each peptide is administered at each dose (0.5 ml). In one embodiment 100 to 2000 mcg of each peptide is administered at each dose. In one embodiment, the foregoing dose is administered every other week over a course of 10 weeks (i.e., 6 administrations). In one embodiment, administration is subcutaneous. In one embodiment, an adjuvant is mixed (emulsified) with the vaccine before dosing. In one embodiment 0.5 mL of immunotherapy composition (i.e., 200 mcg of each peptide) is emulsified with 1.0 mL of adjuvant before administration. In another embodiment, the adjuvant is injected at the same site as the vaccine, before or after the immunotherapy composition is injected. In one embodiment, the adjuvant is an emulsion. In one embodiment, the emulsion is a Montanide emulsion. In one embodiment, the Montanide emulsion is the immunologic adjuvant Montanide ISA 51 VG. Optionally, in the practice of the invention, one or more checkpoint inhibitors is also administered to the subject with the immunotherapy composition, as described further below.

As noted above, immunotherapy composition comprising or consisting of the combination of seven WT1 peptides may be administered as an immunogenic composition to elicit an immune response against a WT1 expressing cancer, or in another embodiment, the combination of WT1 peptides may be used to prepare WT1-specific CTLs using in vitro or ex vivo methods, said CTLs upon administration to the patient will be directed against a WT1 expressing cancer. In one embodiment, the combination of at least seven WT1 peptides is used to induce the production of CTLs in vitro, using cells from a cell line, for example. In another embodiment, the combination of at least seven WT1 peptides is used to induce the production of CTLs in a sample of cells taken from the patient, wherein the CTLs induced ex vivo are infused back into the same patient in need thereof. In another embodiment, the combination of at least seven WT1 peptides is used to induce the production of CTLs in a sample of cells taken from a donor, wherein the CTLs induced ex vivo are infused into a patient in need thereof who is not the donor. In another embodiment, a subject who is not the patient in need of therapy, is administered the combination of seven or more WT1 peptides described here in order to induce the formation of CTLs, which are then transferred from the donor to the patient. Each of these embodiments are other aspects of the invention, and sources of WT1 specific cells useful in treating cancer or reducing the incidence of cancer or its relapse as described herein. In any of the foregoing embodiments, CTLs to each of the at least seven WT1 peptides may be prepared individually or in combination. CTLs that are prepared individually can be administered to a subject separately, or may be combined prior to administration to the subject.

In any of the foregoing methods, whether conducting immunotherapy on the patient to induce a CTL response against a WT1 expressing cancer, or obtaining WT1 specific CTLs from a donor, from an in vitro or ex vivo method using immune cells from a cell line, the patient, or a donor who is not the patient, the combined use of a checkpoint inhibitor may optionally embodied herein, whether the methods for treating, reducing the incidence of cancer or its relapse is by immunizing the subject in need thereof with the combination of seven or more WT1 peptides, or producing CTLs in vitro ex vivo or in a donor subject. In any of these methods, the combined use of one or more checkpoint inhibitors may optionally be embodied herein. The one or more checkpoint inhibitor may be administered to the patient that is being immunized with the one or more WT1 peptides. The checkpoint inhibitor may be used in vitro or ex vivo to enhance the formation of WT1 specific CTLs that are subsequently infused into the patient. The one or more checkpoint inhibitors may be used in the donor subject to enhance the formation of WT1 specific CTLs that will then be transferred into the patient. The checkpoint inhibitor may be used in the patient receiving CTLs prepared in vitro, ex vivo, or in a donor, whether or not the in vitro, ex vivo, or donor was also administered a checkpoint inhibitor. In the latter embodiments, the same or different one or more checkpoint inhibitors may be used in the in vitro, ex vivo or donor subject, and in the patient.

Immune checkpoints regulate T cell function in the immune system. T cells play a central role in cell-mediated immunity. Checkpoint proteins interact with specific ligands which send a signal into the T cell and essentially switch off or inhibit T cell function. Cancer cells take advantage of this system by driving high levels of expression of checkpoint proteins on their surface which results in control of the T cells expressing checkpoint proteins on the surface of T cells that enter the tumor microenvironment, thus suppressing the anticancer immune response. As such, inhibition of checkpoint proteins would result in restoration of T cell function and an immune response to the cancer cells. An immune checkpoint inhibitor (or checkpoint inhibitor) is a compound or agent that blocks or inhibits immune checkpoint proteins (i.e., that blocks or inhibits checkpoint receptors or checkpoint receptor ligands). Examples of checkpoint proteins include, but are not limited to, CTLA-4, PD-L1, PD-L2, PD1, B7-H3, B7-H4, BTLA, HVEM, TIM3, GALS, LAG3, VISTA, IDO, KIR, 2B4 (belongs to the CD2 family of molecules and is expressed on all NK cells, and memory CD8⁺ T cells), CD160 (also referred to as BY55), CGEN-15049, CHK 1 and CHK2 kinases, A2aR, and various B-7 family ligands. Programmed Death-1 (PD-1) is a member of the immunoglobulin superfamily (IGSF) of molecules involved in regulation of T cell activation. PD-1 acquired its name ‘programmed death’ when it was identified in 1992 as a gene upregulated in T cell hybridoma undergoing cell death. The structure of PD-1 is composed of one IGSF domain, a transmembrane domain, and an intracellular domain containing an immunoreceptor tyrosine-based inhibitory motif (ITIM) and an immunoreceptor tyrosine-based switch motif (ITSM) [38]. PD-1 has two binding partners: PD-L1 (B7-H1, CD274) and PD-L2 (B7-DC, CD273). PD-L1 is expressed broadly on both hematopoietic and non-hematopoietic lineages [39, 40]. It is found on T cell, B cells, macrophages, NK cells, DCs, and mast cells as well as in peripheral tissues [41, 42]. PD-1 engagement represents one means by which tumors evade immunosurveillance and clearance [43]. Blockade of the PD-1 pathway has been demonstrated by nivolumab, which shows activity in immunocompetent mouse cancer models [44].

Non-limiting examples of checkpoint inhibitors include small molecules, peptides, and antibodies. Non-limiting examples of antibodies include nivolumab (OPDIVO), pembrolizumab (KEYTRUDA), pidilizumab (CT-011), MEDI0680 (AMP-514), AMP-224, AUNP-12, BMS 936559, atezolizumab (MPDL3280A), durvalumab (MEDI4736), avelumab (MSB0010718C), BMS935559 (MDX-1105), rHIgM12B7, BMS-986016, GSK2831781, IMP321, lirilumab (BMS-986015), IPH2101 (1-7F9), Indoximod (NLG 9189), NLG 919, INCB024360, PF-05082566, Urelumab (BMS-663513), and MEDI6469.

Nivolumab (OPDIVO) is a fully human IgG4 monoclonal antibody targeted against PD-1 receptor on activated T and B lymphocytes[47]. Pembrolizumab (KEYTRUDA) is another non-limiting example of an antibody that targets PD-1. Other compounds and agents that block, inhibit or target checkpoint proteins include compounds undergoing testing and not yet available on the market. The invention is not limited by the specific checkpoint inhibitor. Non-limiting examples of checkpoint inhibitors that may be used are listed in Table 1.

TABLE 1
Examples of Checkpoint Inhibitors

| Name | Class of Agent | Target |
|---|---|---|
| Ipilumumab (a.k.a. | IgG1 human mAb | Cytotoxic |
| MDX-010; MDX-101; | | T-lymphocyte |
| BMS-734016; | | antigen 4 |
| marketed as Yervoy) | | (CTLA-4) |
| Tremelimumab (a.k.a. | IgG2 human mAb | CTLA-4 |
| ticilimumab; CP-675-206) | | |
| Nivolumab (a.k.a. | IgG4 human mAb | Programmed death-1 |
| ONO-4538; BMS- | | (PD-1) |
| 936558; MDX1106; | | |
| marketed as Opdivo) | | |
| Pembrolizumab | IgG4 humanized mAb | PD-1 |
| (a.k.a., MK-3475; | | |
| lambrolizumab; | | |
| marketed as Keytruda) | | |
| Pidlizumab | IgG1 humanized mAb | PD-1 |
| (a.k.a. CT-011) | | |
| MEDI0680 | IgG4 humanized mAb | PD-1 |
| (a.k.a. AMP-514) | | |
| AMP-224 | Fc-PD-L2 fusion | PD-1 |
| | protein | |
| AUNP-12 | Branched, 29-amino | PD-1 |
| | acid peptide | |
| BMS-936559 | IgG4 human mAb | Programmed death |
| | | ligand-1 (PD-L1) |
| Atezolizumab (a.k.a. | IgG1 humanized mAb | PD-Ll |
| MPDL3280A; RG7446) | | |
| Durvalumab | IgG1 human mAb | PD-Ll |
| (a.k.a. MEDI4736) | | |
| Avelumab | IgG1 human mAb | PD-Ll |
| (a.k.a. MSB0010718C) | | |
| BMS935559 | IgG4 human mAb | PD-Ll |
| (a.k.a. MDX-1105) | | |
| rHIgMl2B7 | IgM human mAb | Programmed death |
| | | ligand-2 (PD-L2) |
| BMS-986016 | mAB | Lymphocyte |
| | | activation |
| | | gene-3 (LAG-3; |
| | | a.k.a. CD223) |
| GSK2831781 | Humanized | LAG-3 |
| | afuscated mAb | |
| IMP321 | Soluble LAG-3 | LAG-3 |
| Lirilumab | IgG4 human mAb | Killer cell |
| (a.k.a. BMS-986015) | | immunoglobulin- |
| | | like receptor (KIR) |
| IPH2101 | Anti-inhibitor | KIR |
| (a.k.a. 1-7F9) | monoclonal Ab | |
| Indoximod | Small molecule (D | Indoleamine- |
| (a.k.a. NLG 9189; | isomer of 1- | 2,3-dioxygenase 1 |
| CAS #110117-83-4) | methyl-tryptophan) | (IDO1) |
| NLG 919 | Small molecule | IDO1 |
| (CAS #1402836-58-1) | | |
| INCB024360 | Small molecule | IDO1 |
| (CAS #914471-09-3) | | |
| PF-05082566 | IgG2 human mAB | 4-1BB (a.k.a. CD137) |
| Urelumab | IgG4 humanized mAb | 4-1BB |
| (a.k.a. BMS-663513) | | |
| MEDI6469 | IgG1 mouse | OX40 (a.k.a. CD134) |
| | anti-human Ab | |

In one embodiment, a combination of two or more checkpoint inhibitors is administered to the subject. In one embodiment, the combination of checkpoint inhibitors is selected from among those in Table 1. The two or more checkpoint inhibitors can be administered simultaneously or consecutively with respect to one another and with respect to the one or more WT1 peptides. In a further embodiment, the combination of two or more checkpoint inhibitors target two different checkpoint proteins, such as PD-1 (e.g., nivolumab or other PD-1 inhibitor) and CTLA-4 (e.g., ipilumumab or other CTLA-4 inhibitor), are administered to the subject simultaneously or consecutively with respect to one another and with respect to the one or more WT1 peptides. In one embodiment, the combination of two or more checkpoint inhibitors target two or more different checkpoint proteins from among: CTLA-4, PD-L1, PD-L2, PD1, B7-H3, B7-H4, BTLA, HVEM, TIM3, GALS, LAG3, VISTA, KIR, 2B4, CD160, CGEN-15049, CHK 1 kinase, CHK2 kinase, A2aR, and B-7 family ligands. In one embodiment, the combination of two or more checkpoint inhibitors targeting two or more different checkpoint proteins is selected from among those in Table 1.

The dose level, frequency of dosing, duration of dosing and other aspects of administration of the checkpoint inhibitor may be optimized in accordance with the patient's clinical presentation, duration or course of the disease, comorbidities, and other aspects of clinical care. The invention is not so limiting with regard to the particular aspects of the checkpoint inhibitor component of the methods embodied herein.

In one embodiment, a nivolumab dose and schedule selection of 3 mg/kg every 2 weeks over a course of 12 weeks. In one embodiment, administration is intravenous. In one embodiment, the course of checkpoint inhibitor administration is concurrent with that of the WT1 vaccine administration. In one embodiment the course of checkpoint inhibitor administration overlaps with that of the WT1 vaccine administration. In one embodiment the course of checkpoint inhibitor administration starts at about the same time as the course of the WT1 vaccine administration.

In one embodiment, the immunotherapy composition comprises 200 mcg of each of the peptides YMFPNAPYL (SEQ ID NO: 124), RSDELVRHHNMHQRNMTKL (SEQ ID NO: 1), PGCNKRYFKLSHLQMHSRKHTG (SEQ ID NO: 2), SGQAYMFPNAPYLPSCLES (SEQ ID NO: 125), NLMNLGATL (SEQ ID NO: 21), WNLMNLGATLKGVAA (SEQ ID NO: 26), and WNYMNLGATLKGVAA (SEQ ID NO: 205) combined in a total volume of 0.5 ml emulsified with 1.0 mL Montanide ISA 51 VG and administered subcutaneously every 2 weeks for 6 administrations; and nivolumab, 3 mg/kg, is administered intravenously by 60 minute infusion every two weeks for 7 administrations, starting at the same time as the WT1 immunotherapy.

In one embodiment, methods are embodied herein in which the combination of seven or more WT1 peptides, and optionally, the one or more checkpoint inhibitors are each administered to a subject according to a schedule that maximally benefits the patient. The one or more WT1 peptide and the one or more checkpoint inhibitor are therefore not necessarily administered at the same time or even in the same composition or each for the same duration. Each WT1 peptide, or combination of WT1 peptides, may be administered in accordance with a particular schedule, as may be each checkpoint inhibitor. In one non-limiting embodiment, the combination of seven or more WT1 peptides and one or more checkpoint inhibitors are present in the same composition.

As noted herein, the dose level and dosing schedule including frequency and duration of the WT1 peptide or peptides (separately or administered together) and that of the one or more checkpoint inhibitors (administered separately or together), the route of administration, and other aspects of administration are optimized for maximal benefit to the patient subject. These same aspects are also considered when a donor subject is the recipient of the WT1 peptide or peptides and the checkpoint inhibitor or inhibitors for the purpose of generating WT1 specific CTLs to administer to the patient.

In one embodiment, compositions are provided containing the combination of at least seven WT1 peptides and at least one checkpoint inhibitor. In one embodiment, the WT1 peptide or peptides in the composition are among those disclosed herein. In one embodiment, the checkpoint inhibitor is among those disclosed herein. In one embodiment the composition comprises the checkpoint inhibitor nivolumab, pembrolizumab, or the combination thereof. The composition may further comprise an excipient, diluent or carrier. The composition may also comprise one or more adjuvants.

The foregoing embodiments provide improved methods of treating, reducing the incidence of, and inducing immune responses against a WT1-expressing cancer, and compositions useful for the same purposes. Other aspects of the invention are described further below.

In one embodiment, a modified WT1 peptide has one or more altered amino acids, referred to herein as a mutated WT1 peptide. In one embodiment the mutated WT1 peptide comprise: (a) a binding motif of a human leukocyte antigen (HLA) Class II molecule; and (b) a binding motif of an HLA class I molecule comprising a point mutation in one or more anchor residues of the binding motif of an HLA class I molecule. In another embodiment, the peptide is 11 or more amino acids in length. In certain other embodiments, the peptide is 11-22, 11-30, 16-22 or 16-30 amino acids in length. In another embodiment, the point mutation is in 1-3 anchor residues of the HLA class I molecule binding motif. In another embodiment, the point mutation is in 1 anchor residue of the HLA class I molecule binding motif. In another embodiment, the point mutation is in 2 anchor residues of the HLA class I molecule binding motif. In another embodiment, the point mutation is in 1-2 anchor residues of the HLA class I molecule binding motif. In another embodiment, the point mutation is in 2-3 anchor residues of the HLA class I molecule binding motif. In another embodiment, the point mutation is in 1-4 anchor residues of the HLA class I molecule binding motif. Each possibility represents a separate embodiment of the present invention.

In another embodiment, the present invention provides a method of treating a subject with a WT1-expressing cancer, the method comprising administering to the subject the combination of at least seven WT1 peptides and, optionally, at least one checkpoint inhibitor, thereby treating a subject with a WT1-expressing cancer.

In another embodiment, the present invention provides a method of reducing the incidence of a WT1-expressing cancer, or its relapse, in a subject, the method comprising administering to the subject the combination of at least seven WT1 peptides and, optionally, at least one checkpoint inhibitor, thereby reducing the incidence of a WT1-expressing cancer, or its relapse, in a subject.

In another embodiment, the present invention provides a method of inducing formation and proliferation of a WT1 protein-specific CTL, the method comprising contacting a lymphocyte population with the combination of at least seven WT1 peptides and, optionally, at least one checkpoint inhibitor, thereby inducing formation and proliferation of a WT1 protein-specific CTL.

In another embodiment, the present invention provides a method of inducing formation and proliferation of (a) a WT1 protein-specific CD8⁺ lymphocyte; and (b) a CD4⁺ lymphocyte specific for the WT1 protein, the method comprising contacting a lymphocyte population with the combination at least seven WT1 peptides and, optionally, at least one checkpoint inhibitor, thereby inducing formation and proliferation of (a) a WT1 protein-specific CD8⁺ lymphocyte; and (b) a CD4⁺ lymphocyte specific for the WT1 protein.

In one embodiment, the aforementioned methods for treating a WT1 expressing cancer, reducing the incidence of a WT1 expressing cancer or inducing the formation and proliferation of a WT1 protein specific T cell response, are achieved with greater effect than if such methods employ only the combination of at least seven WT1 peptides alone or the checkpoint inhibitor(s) alone. In one embodiment, the course of administration of the WT1 immunotherapy and the course of administration of the one or more checkpoint inhibitors are concurrent, overlap, or are contemporaneous such that the biological response to the vaccine is enhanced by the administration of the one or more checkpoint inhibitors. Contemporaneous administration embraces a course of WT1 immunotherapy to induce WT1 specific CTLs, and administration of the one or more checkpoint inhibitor to enhance the activity of the CTLs against the cancer. In one embodiment, the course of WT1 vaccine administration can end before the course of checkpoint inhibitor therapy begins, insofar as the effectiveness of the CTLs elicited by the WT1 immunotherapy administration is enhanced by the checkpoint inhibitor therapy. In one embodiment, the first administration of checkpoint inhibitor therapy is on the same day as the last WT1 immunotherapy administration. In one embodiment the end of WT1 immunotherapy and the start of checkpoint inhibitor therapy is separated by from 1-7 days or from 1-4 weeks.

As noted herein, the one or more additional WT1 peptide(s) may be native fragments, or contiguous amino acid sequences, of the WT1 protein, or they may have one or more modifications of the amino acid sequence to enhance immunogenicity or any other beneficial property to the peptide and the development of immunity to a WT1 expressing cancer. In certain embodiments, one or amino acids are changed to enhance immunogenicity. In one embodiment, the methods of use employ an isolated, mutated WT1 peptide, comprising: (a) a binding motif of a human leukocyte antigen (HLA) Class II molecule; and (b) a binding motif of an HLA class I molecule, having a point mutation in 1 or more anchor residues of the binding motif of an HLA class I molecule. In another embodiment, the peptide is 11 or more aa in length. Each possibility represents a separate embodiment of the present invention.

The “point mutation,” in another embodiment, indicates that the fragment is mutated with respect to the native sequence of the protein, thus creating the HLA class I molecule binding motif. In another embodiment, the “point mutation” strengthens the binding capacity of an HLA class I molecule binding motif present in the native sequence. Each possibility represents a separate embodiment of the methods of use of present invention.

In another embodiment, the point mutation is in 1-3 anchor residues of the HLA class I molecule binding motif. In another embodiment, the point mutation is in 1 anchor residue of the HLA class I molecule binding motif. In another embodiment, the point mutation is in 2 anchor residues of the HLA class I molecule binding motif. In another embodiment, the point mutation is in 1-2 anchor residues of the HLA class I molecule binding motif. In another embodiment, the point mutation is in 2-3 anchor residues of the HLA class I molecule binding motif. In another embodiment, the point mutation is in 1-4 anchor residues of the HLA class I molecule binding motif. Each possibility represents a separate embodiment of the present invention.

In another embodiment, a peptide of the present invention is 11-453 amino acids (AA) in length. In another embodiment, the length is 12-453 AA. In another embodiment, the length is 13-453 AA. In another embodiment, the length is 14-453 AA. In another embodiment, the length is 15-453 AA. In another embodiment, the length is 16-453 AA. In another embodiment, the length is 17-453 AA. In another embodiment, the length is 18-453 AA. In another embodiment, the length is 19-453 AA. In another embodiment, the length is 20-453 AA.

In another embodiment, the length is 11-449 AA. In another embodiment, the length is 12-449 AA. In another embodiment, the length is 13-449 AA. In another embodiment, the length is 14-449 AA. In another embodiment, the length is 15-449 AA. In another embodiment, the length is 16-449 AA. In another embodiment, the length is 17-449 AA. In another embodiment, the length is 18-449 AA. In another embodiment, the length is 19-449 AA. In another embodiment, the length is 20-449 AA.

In another embodiment, the length is 11-30 AA. In another embodiment, the length is 16-22 AA. In another embodiment, the length is 19 AA. In another embodiment, the peptide is 15-23 AA in length. In another embodiment, the length is 15-24 AA. In another embodiment, the length is 15-25 AA. In another embodiment, the length is 15-26 AA. In another embodiment, the length is 15-27 AA. In another embodiment, the length is 15-28 AA. In another embodiment, the length is 14-30 AA. In another embodiment, the length is 14-29 AA. In another embodiment, the length is 14-28 AA. In another embodiment, the length is 14-26 AA. In another embodiment, the length is 14-24 AA. In another embodiment, the length is 14-22 AA. In another embodiment, the length is 14-20 AA. In another embodiment, the length is 16-30 AA. In another embodiment, the length is 16-28 AA. In another embodiment, the length is 16-26 AA. In another embodiment, the length is 16-24 AA. In another embodiment, the length is 16-22 AA. In another embodiment, the length is 18-30 AA. In another embodiment, the length is 18-28 AA. In another embodiment, the length is 18-26 AA. In another embodiment, the length is 18-24 AA. In another embodiment, the length is 18-22 AA. In another embodiment, the length is 18-20 AA. In another embodiment, the length is 20-30 AA. In another embodiment, the length is 20-28 AA. In another embodiment, the length is 20-26 AA. In another embodiment, the length is 20-24 AA. In another embodiment, the length is 22-30 AA. In another embodiment, the length is 22-28 AA. In another embodiment, the length is 22-26 AA. In another embodiment, the length is 24-30 AA. In another embodiment, the length is 24-28 AA. In another embodiment, the length is 24-26 AA.

In another embodiment, a peptide useful for the methods and compositions of the present invention is longer than the minimum length for binding to an HLA class II molecule, which is, in another embodiment, about 12 AA. In another embodiment, increasing the length of the HLA class II-binding peptide enables binding to more than one HLA class II molecule. In another embodiment, increasing the length enables binding to an HLA class II molecule whose binding motif is not known. In another embodiment, increasing the length enables binding to an HLA class I molecule. In another embodiment, the binding motif of the HLA class I molecule is known. In another embodiment, the binding motif of the HLA class I molecule is not known. Each possibility represents a separate embodiment of the present invention.

Each of the above peptide lengths represents a separate embodiment of the present invention.

HLA molecules, known in another embodiment as major histocompatibility complex (MHC) molecules, bind peptides and present them to immune cells. Thus, in another embodiment, the immunogenicity of a peptide is partially determined by its affinity for HLA molecules. HLA class I molecules interact with CD8 molecules, which are generally present on cytotoxic T lymphocytes (CTL). HLA class II molecules interact with CD4 molecules, which are generally present on helper T lymphocytes.

In another embodiment, a peptide of the present invention is immunogenic. In another embodiment, the term “immunogenic” refers to an ability to stimulate, elicit or participate in an immune response. In another embodiment, the immune response elicited is a cell-mediated immune response. In another embodiment, the immune response is a combination of cell-mediated and humoral responses.

In another embodiment, T cells that bind to the HLA molecule-peptide complex become activated and induced to proliferate and lyse cells expressing a protein comprising the peptide. T cells are typically initially activated by “professional” antigen presenting cells (“APC”; e.g. dendritic cells, monocytes, and macrophages), which present costimulatory molecules that encourage T cell activation rather than anergy or apoptosis. In another embodiment, the response is heteroclitic, as described herein, such that the CTL lyses a neoplastic cell expressing a protein which has an AA sequence homologous to a peptide of this invention, or a different peptide than that used to first stimulate the T cell.

In another embodiment, an encounter of a T cell with a peptide of this invention induces its differentiation into an effector and/or memory T cell. Subsequent encounters between the effector or memory T cell and the same peptide, or, in another embodiment, with a heteroclitic peptide of this invention, leads to a faster and more intense immune response. Such responses are gauged, in another embodiment, by measuring the degree of proliferation of the T cell population exposed to the peptide. In another embodiment, such responses are gauged by any of the methods enumerated herein below.

In another embodiment, as described herein, the subject is exposed to a peptide, or a composition/cell population comprising a peptide of this invention, which differs from the native protein expressed, wherein subsequently a host immune response cross-reactive with the native protein/antigen develops.

In another embodiment, peptides, compositions, and vaccines of this invention stimulate an immune response that results in tumor cell lysis. In all of the foregoing embodiments, the concurrent use of a checkpoint inhibitor enhances the immune response against the tumor.

In another embodiment, the HLA class I molecule binding motif of a peptide of the present invention is contained within the HLA class II molecule binding motif of the peptide. In another embodiment, the HLA class I molecule binding motif overlaps with the HLA class II molecule binding motif. In another embodiment, the HLA class I molecule binding motif does not overlap with the HLA class II molecule binding motif. Each possibility represents a separate embodiment of the present invention.

The HLA class II molecule whose binding motif is contained in a peptide of the present invention is, in another embodiment, an HLA-DR molecule. In another embodiment, the HLA class II molecule is an HLA-DP molecule. In another embodiment, the HLA class II molecule is an HLA-DQ molecule.

In another embodiment, the HLA class II molecule is an HLA-DRB molecule. In another embodiment, the HLA class II molecule is DRB101. In another embodiment, the HLA class II molecule is DRB301. In another embodiment, the HLA class II molecule is DRB401. In another embodiment, the HLA class II molecule is DRB701. In another embodiment, the HLA class II molecule is DRB1101. In another embodiment, the HLA class II molecule is DRB1501. In another embodiment, the HLA class II molecule is any other HLA-DRB molecule known in the art. In another embodiment, the HLA class II molecule is an HLA-DRA molecule. In another embodiment, the HLA class II molecule is an HLA-DQA1 molecule. In another embodiment, the HLA class II molecule is an HLA-DQB1 molecule. In another embodiment, the HLA class II molecule is an HLA-DPA1 molecule. In another embodiment, the HLA class II molecule is an HLA-DPB1 molecule. In another embodiment, the HLA class II molecule is an HLA-DMA molecule. In another embodiment, the HLA class II molecule is an HLA-DMB molecule. In another embodiment, the HLA class II molecule is an HLA-DOA molecule. In another embodiment, the HLA class II molecule is an HLA-DOB molecule. In another embodiment, the HLA class II molecule is any other HLA class II-molecule known in the art.

In another embodiment, a peptide of the present invention binds to 2 distinct HLA class II molecules. In another embodiment, the peptide binds to three distinct HLA class II molecules. In another embodiment, the peptide binds to four distinct HLA class II molecules. In another embodiment, the peptide binds to five distinct HLA class II molecules. In another embodiment, the peptide binds to six distinct HLA class II molecules. In another embodiment, the peptide binds to more than six distinct HLA class II molecules.

In another embodiment, the HLA class II molecules that are bound by a peptide of the present invention are encoded by two or more distinct alleles at a given HLA class II locus. In another embodiment, the HLA class II molecules are encoded by three distinct alleles at a locus. In another embodiment, the HLA class II molecules are encoded by four distinct alleles at a locus. In another embodiment, the HLA class II molecules are encoded by five distinct alleles at a locus. In another embodiment, the HLA class II molecules are encoded by six distinct alleles at a locus. In another embodiment, the HLA class II molecules are encoded by more than six distinct alleles at a locus.

In another embodiment, the HLA class II molecules bound by the peptide are encoded by HLA class II genes at two distinct loci. In another embodiment, the HLA class II molecules are encoded by HLA class II genes at 2 or more distinct loci. In another embodiment, the HLA class II molecules are encoded by HLA class II genes at 3 distinct loci. In another embodiment, the HLA class II molecules are encoded by HLA class II genes at 3 or more distinct loci. In another embodiment, the HLA class II molecules are encoded by HLA class II genes at 4 distinct loci. In another embodiment, the HLA class II molecules are encoded by HLA class II genes at 4 or more distinct loci. In another embodiment, the HLA class II molecules are encoded by HLA class II genes at 5 distinct loci. In another embodiment, the HLA class II molecules are encoded by HLA class II genes at 5 or more distinct loci. In another embodiment, the HLA class II molecules are encoded by HLA class II genes at 6 distinct loci. In another embodiment, the HLA class II molecules are encoded by HLA class II genes at 6 or more distinct loci. In another embodiment, the HLA class II molecules are encoded by HLA class II genes at more than 6 distinct loci. Each possibility represents a separate embodiment of the present invention.

In another embodiment, a peptide of the present invention binds to 2 distinct HLA-DRB molecules. In another embodiment, the peptide binds to three distinct HLA-DRB molecules. In another embodiment, the peptide binds to four distinct HLA-DRB molecules. In another embodiment, the peptide binds to five distinct HLA-DRB molecules. In another embodiment, the peptide binds to six distinct HLA-DRB molecules. In another embodiment, the peptide binds to more than six distinct HLA-DRB molecules.

In another embodiment, the HLA class II molecules bound by the WT1 peptide are encoded by HLA class II genes at 2 distinct loci. In another embodiment, the HLA molecules bound are encoded by HLA class II genes at 2 or more distinct loci. In another embodiment, the HLA molecules bound are encoded by HLA class II genes at 3 distinct loci. In another embodiment, the HLA molecules bound are encoded by HLA class II genes at 3 or more distinct loci. In another embodiment, the HLA molecules bound are encoded by HLA class II genes at 4 distinct loci. In another embodiment, the HLA molecules bound are encoded by HLA class II genes at 4 or more distinct loci. In another embodiment, the HLA molecules bound are encoded by HLA class II genes at more than 4 distinct loci. In other embodiments, the loci are selected from HLA-DRB loci. In another embodiment, the HLA class II-binding peptide is an HLA-DRA binding peptide. In another embodiment, the peptide is an HLA-DQA1 binding peptide. In another embodiment, the peptide is an HLA-DQB1 binding peptide. In another embodiment, the peptide is an HLA-DPA1 binding peptide. In another embodiment, the peptide is an HLA-DPB1 binding peptide. In another embodiment, the peptide is an HLA-DMA binding peptide. In another embodiment, the peptide is an HLA-DMB binding peptide. In another embodiment, the peptide is an HLA-DOA binding peptide. In another embodiment, the peptide is an HLA-DOB binding peptide. In another embodiment, the peptide binds to any other HLA class II molecule known in the art. Each possibility represents a separate embodiment of the present invention.

In another embodiment, a peptide of the present invention binds to HLA-DRB molecules that are encoded by 2 distinct HLA-DRB alleles selected from DRB 101, DRB 301, DRB 401, DRB 701, DRB 1101, and DRB 1501. In another embodiment, the peptide binds to HLA-DRB molecules encoded by 3 distinct HLA-DRB alleles selected from DRB 101, DRB 301, DRB 401, DRB 701, DRB 1101, and DRB 1501. In another embodiment, the peptide binds to HLA-DRB molecules encoded by 4 distinct HLA-DRB alleles selected from DRB 101, DRB 301, DRB 401, DRB 701, DRB 1101, and DRB 1501. In another embodiment, the peptide binds to HLA-DRB molecules encoded by 5 distinct HLA-DRB alleles selected from DRB 101, DRB 301, DRB 401, DRB 701, DRB 1101, and DRB 1501. In another embodiment, the peptide binds to HLA-DRB molecules encoded by each of the following HLA-DRB alleles: DRB 101, DRB 301, DRB 401, DRB 701, DRB 1101, and DRB 1501. Each possibility represents a separate embodiment of the present invention.

Each of the above HLA class II molecule, types, classes, and combinations thereof represents a separate embodiment of the present invention.

The HLA class I molecule whose binding motif is contained in a peptide of the present invention is, in another embodiment, an HLA-A molecule. In another embodiment, the HLA class I molecule is an HLA-B molecule. In another embodiment, the HLA class I molecule is an HLA-C molecule. In another embodiment, the HLA class I molecule is an HLA-A0201 molecule. In another embodiment, the molecule is HLA A1. In another embodiment, the HLA class I molecule is HLA A2. In another embodiment, the HLA class I molecule is HLA A2.1. In another embodiment, the HLA class I molecule is HLA A3. In another embodiment, the HLA class I molecule is HLA A3.2. In another embodiment, the HLA class I molecule is HLA A11. In another embodiment, the HLA class I molecule is HLA A24. In another embodiment, the HLA class I molecule is HLA B7. In another embodiment, the HLA class I molecule is HLA B27. In another embodiment, the HLA class I molecule is HLA B8. Each possibility represents a separate embodiment of the present invention.

In another embodiment, the HLA class I molecule-binding WT1 peptide of methods and compositions of the present invention binds to a superfamily of HLA class I molecules. In another embodiment, the superfamily is the A2 superfamily. In another embodiment, the superfamily is the A3 superfamily. In another embodiment, the superfamily is the A24 superfamily. In another embodiment, the superfamily is the B7 superfamily. In another embodiment, the superfamily is the B27 superfamily. In another embodiment, the superfamily is the B44 superfamily. In another embodiment, the superfamily is the C1 superfamily. In another embodiment, the superfamily is the C4 superfamily. In another embodiment, the superfamily is any other superfamily known in the art. Each possibility represents a separate embodiment of the present invention.

In another embodiment, an HLA class I molecule binding motif of a peptide of the present invention exhibits an increased affinity for the HLA class I molecule, relative to the unmutated counterpart of the peptide. In another embodiment, the point mutation increases the affinity of the isolated, mutated WT1 peptide for the HLA class I molecule. In another embodiment, the increase in affinity is relative to the affinity (for the same HLA class I molecule) of the isolated, unmutated WT1 peptide wherefrom the isolated, mutated WT1 peptide was derived. Each possibility represents a separate embodiment of the present invention.

In another embodiment, an HLA class I molecule-binding WT peptide of methods and compositions of the present invention has a length of 9-13 AA. In another embodiment, the length is 8-13 AA. In another embodiment, the peptide has any of the lengths of a peptide of the present invention enumerated herein.

In another embodiment, the HLA class I molecule-binding WT peptide has length of 8 AA. In another embodiment, the peptide has length of 9 AA. In another embodiment, the peptide has length of 10 AA. As provided herein, native and heteroclitic peptides of 9-10 AA exhibited substantial binding to HLA class I molecules and ability to elicit cytokine secretion and cytolysis by CTL.

In another embodiment, an HLA class I molecule-binding WT1 peptide embedded within a WT1 peptide of the present invention has 1 of the above lengths. Each possibility represents a separate embodiment of the present invention. In one embodiment, the WT1 peptide is a peptide of longer length than an HLA class I molecule-binding WT1 peptide. The longer length peptide is degraded by cells to the appropriate length to be presented by a HLA class 1 molecule.

In another embodiment, the HLA class I molecule that is bound by the HLA class I molecule-binding WT1 peptide is an HLA-A molecule. In another embodiment, the HLA class I-molecule is an HLA-A2 molecule. In another embodiment, the HLA class I-molecule is an HLA-A3 molecule. In another embodiment, the HLA class I-molecule is an HLA-A11 molecule. In another embodiment, the HLA class I-molecule is an HLA-B8 molecule. In another embodiment, the HLA class I-molecule is an HLA-0201 molecule. In another embodiment, the HLA class I-molecule binds any other HLA class I molecule known in the art. Each possibility represents a separate embodiment of the present invention.

In another embodiment, a peptide of the present invention retains ability to bind multiple HLA class II molecules, as exhibited by the isolated WT1 peptide wherefrom the peptide of the present invention was derived.

In all of the aspects herein, the one or more WT1 peptides useful in the vaccine herein or for generating CTLs in vitro, ex vivo or in a donor, the selection of the peptide or peptides sequences, whether native or modified, to match the HLA type(s) of the patient or donor is embodied herein.

The WT1 molecule from which a peptide of the present invention may be derived has, in another embodiment, the sequence:

MGSDVRDLNALLPAVPSLGGGGGCALPVSGAAQWAPVLDFAPPGASAYG
SLGGPAPPPAPPPPPPPPPHSFIKQEPSWGGAEPHEEQCLSAFTVHFSG
QFTGTAGACRYGPFGPPPPSQASSGQARMFPNAPYLPSCLESQPAIRNQ
GYSTVTFDGTPSYGHTPSHHAAQFPNHSFKHEDPMGQQGSLGEQQYSVP
PPVYGCHTPTDSCTGSQALLLRTPYSSDNLYQMTSQLECMTWNQMNLGA
TLKGVAAGSSSSVKWTEGQSNHSTGYESDNHTTPILCGAQYRIHTHGVF
RGIQDVRRVPGVAPTLVRSASETSEKRPFMCAYPGCNKRYFKLSHLQMH
SRKHTGEKPYQCDFKDCERRFSRSDQLKRHQRRHTGVKPFQCKTCQRKF
SRSDHLKTHTRTHTGKTSEKPFSCRWPSCQKKFARSDELVRHHNMHQRN
MTKLQLAL (SEQ ID NO: 199; GenBank Accession
number AY245105).

In another embodiment, the WT1 molecule has the sequence:

AAEASAERLQGRRSRGASGSEPQQMGSDVRDLNALLPAVPSLGGGGGCA
LPVSGAAQWAPVLDFAPPGASAYGSLGGPAPPPAPPPPPPPPPHSFIKQ
EPSWGGAEPHEEQCLSAFTVHFSGQFTGTAGACRYGPFGPPPPSQASSG
QARMFPNAPYLPSCLESQPAIRNQGYSTVTFDGTPSYGHTPSHHAAQFP
NHSFKHEDPMGQQGSLGEQQYSVPPPVYGCHTPTDSCTGSQALLLRTPY
SSDNLYQMTSQLECMTWNQMNLGATLKGHSTGYESDNHTTPILCGAQYR
IHTHGVFRGIQDVRRVPGVAPTLVRSASETSEKRPFMCAYPGCNKRYFK
LSHLQMHSRKHTGEKPYQCDFKDCERRFSRSDQLKRHQRRHTGVKPFQC
KTCQRKFSRSDHLKTHTRTHTGEKPFSCRWPSCQKKFARSDELVRHHNM
HQRNMTKLQLAL (SEQ ID NO: 200; GenBank Accession
number NM_000378).

In another embodiment, the WT1 molecule has the sequence:

MQDPASTCVPEPASQHTLRSGPGCLQQPEQQGVRDPGGIWAKLGAAEAS
AERLQGRRSRGASGSEPQQMGSDVRDLNALLPAVPSLGGGGGCALPVSG
AAQWAPVLDFAPPGASAYGSLGGPAPPPAPPPPPPPPPHSFIKQEPSWG
GAEPHEEQCLSAFTVHFSGQFTGTAGACRYGPFGPPPPSQASSGQARMF
PNAPYLPSCLESQPAIRNQGYSTVTFDGTPSYGHTPSHHAAQFPNHSFK
HEDPMGQQGSLGEQQYSVPPPVYGCHTPTDSCTGSQALLLRTPYSSDNL
YQMTSQLECMTWNQMNLGATLKGVAAGSSSSVKWTEGQSNHSTGYESDN
HTTPILCGAQYRIHTHGVFRGIQDVRRVPGVAPTLVRSASETSEKRPFM
CAYPGCNKRYFKLSHLQMHSRKHTGEKPYQCDFKDCERRFSRSDQLKRH
QRRHTGVKPFQCKTCQRKFSRSDHLKTHTRTHTGEKPFSCRWPSCQKKF
ARSDELVRHEINMHQRNMTKLQLAL (SEQ ID NO: 201;
GenBank Accession number NP_077742).

In another embodiment, the WT1 molecule comprises the sequence:

(SEQ ID NO: 202)
MGHHHHHHHHHHSSGHIEGRHMRRVPGVAPTLVRSASETSEKRPFMCAY
PGCNKRYFKLSHLQMEISRKHTGEKPYQCDFKDCERRFFRSDQLKRHQR
RHTGVKPFQCKTCQRKFSRSDHLKTHTRTHTGEKPFSCRWPSCQKKFAR
SDELVRHEINMHQRNMTKLQLAL.

In other embodiments, the WT1 protein comprises one of the sequences set forth in one of the following GenBank sequence entries: NM_024426, NM_024425, NM_024424, NM_000378, S95530, D13624, D12496, D12497, AH003034, or X77549. In other embodiments, the WT1 protein has one of the sequences set forth in one of the above GenBank sequence entries. In another embodiment, the WT1 protein is any WT1 protein known in the art. In another embodiment, the WT1 protein has any other WT1 sequence known in the art.

In another embodiment, a peptide useful for the purposes of the present invention is derived from a fragment of a WT1 protein. In another embodiment, the process of derivation comprises introduction of the point mutation in the anchor residues of the HLA class I molecule binding motif. In another embodiment, the process of derivation consists of introduction of the point mutation in the anchor residues of the HLA class I molecule binding motif. In another embodiment, a peptide of the present invention differs from the corresponding fragment of a WT1 protein only by the point mutation in the HLA class I molecule binding motif anchor residue. In another embodiment, an HLA class I molecule binding motif of a peptide of the present invention differs from the corresponding WT1 sequence only by the point mutation in the anchor residue. Each possibility represents a separate embodiment of the present invention.

In another embodiment, the process of derivation of a peptide of the present invention further comprises one or more modifications of an amino acid (AA) to an AA analogue. In another embodiment, the process of derivation further comprises a modification of one or more peptide bond connecting two or more of the AA. In another embodiment, the AA analogue or peptide bond modification is one of the AA analogues or peptide bond modifications enumerated below. Each possibility represents a separate embodiment of the present invention.

The unmutated fragment of a WT1 protein wherefrom a peptide of the present invention (the “counterpart” in the wild-type sequence) is derived, in another embodiment, has the sequence SGQARMFPNAPYLPSCLES (SEQ ID NO: 5). In another embodiment, the unmutated WT1 fragment has the sequence QARMFPNAPYLPSCL (SEQ ID NO:6). In another embodiment, the unmutated WT1 fragment has the sequence LVRHHNMHQRNMTKL (SEQ ID NO:3). In another embodiment, the unmutated WT1 fragment has the sequence RSDELVRHHNMHQRNMTKL (SEQ ID NO:1). In another embodiment, the unmutated WT1 fragment has the sequence NKRYFKLSHLQMHSR (SEQ ID NO:4). In another embodiment, the unmutated WT1 fragment has the sequence PGCNKRYFKLSHLQMHSRKHTG (SEQ ID NO:2). In another embodiment, the unmutated WT1 fragment is any other WT1 fragment that contains an HLA class II molecule binding motif. In another embodiment, the unmutated WT1 fragment is any other WT1 fragment that contains an HLA-DR molecule binding motif. In another embodiment, the unmutated WT1 fragment contains multiple HLA-DR molecule binding motifs. In another embodiment, the unmutated WT1 fragment is any other WT1 fragment that contains an HLA-DRB molecule binding motif. In another embodiment, the unmutated WT1 fragment contains multiple HLA-DRB molecule binding motifs. In another embodiment, a peptide of the present invention differs from its counterpart only in the point mutation that it contains. In another embodiment, a peptide of the present invention differs from its counterpart only in a mutation in HLA class I anchor residue(s). Each possibility represents a separate embodiment of the present invention.

In another embodiment, a peptide of the present invention retains the ability to bind an HLA class II molecule, as exhibited by the unmutated WT1 fragment wherefrom the peptide was derived. In another embodiment, a peptide of the present invention retains ability to bind multiple HLA class II molecules, as exhibited by the unmutated WT1 fragment. Each possibility represents a separate embodiment of the present invention.

In another embodiment, the present invention provides an isolated peptide comprising the AA sequence GATLKGVAAGSSSSVKWT (SEQ ID NO:203) and LKGVAAGSSSSVKWT (SEQ ID NO:204).

“Peptide,” in another embodiment of methods and compositions of the present invention, refers to a compound of subunit AA connected by peptide bonds. In another embodiment, the peptide comprises an AA analogue. In another embodiment, the peptide is a peptidomimetic. In another embodiment, a peptide of the present invention comprises one of the AA analogues enumerated below. The subunits are, in another embodiment, linked by peptide bonds. In another embodiment, the subunit is linked by another type of bond, e.g. ester, ether, etc. In another embodiment, a peptide of the present invention is one of the types of peptidomimetics enumerated below. Each possibility represents a separate embodiment of the present invention.

In another embodiment, a peptide of methods and compositions of the present invention binds with high affinity to the HLA class I molecule whose binding motif is contained therein. In other embodiments, the HLA class I molecule is any HLA class I molecule enumerated herein. In another embodiment, the peptide binds to the HLA class I molecule with medium affinity. In another embodiment, the peptide binds to the HLA class I molecule with significant affinity. In another embodiment, the peptide binds to the HLA class I molecule with measurable affinity. In another embodiment, the peptide exhibits stable binding to the HLA class I molecule. Each possibility represents a separate embodiment of the present invention.

In another embodiment, a peptide of methods and compositions of the present invention binds with high affinity to the HLA class II molecule whose binding motif is contained therein. In other embodiments, the HLA class II molecule is any HLA class II molecule enumerated herein. In another embodiment, the peptide binds with high affinity to more than 1 HLA class II molecules. In another embodiment, the peptide binds to the HLA class II molecule with medium affinity. In another embodiment, the peptide binds with medium affinity to more than 1 HLA class II molecules. In another embodiment, the peptide binds to the HLA class II molecule with significant affinity. In another embodiment, the peptide binds with significant affinity to more than 1 HLA class II molecules. In another embodiment, the peptide binds to the HLA class II molecule with measurable affinity. In another embodiment, the peptide binds with measurable affinity to more than 1 HLA class II molecules. In another embodiment, the peptide exhibits stable binding to the HLA class II molecule. In another embodiment, the peptide exhibits stable binding to more than 1 HLA class II molecules. Each possibility represents a separate embodiment of the present invention.

In another embodiment, a peptide of methods and compositions of the present invention binds to both an HLA class I molecule and an HLA class II molecule with significant affinity. In another embodiment, the peptide binds to both an HLA class I molecule and an HLA class II molecule with high affinity. In another embodiment, the peptide binds to both an HLA class I molecule and an HLA class II molecule with medium affinity. In another embodiment, the peptide binds to both an HLA class I molecule and an HLA class II molecule with measurable affinity. Each possibility represents a separate embodiment of the present invention.

“Fragment,” in another embodiment, refers to a peptide of 11 or more AA in length. In another embodiment, a peptide fragment of the present invention is 16 or more AA long. In another embodiment, the fragment is 12 or more AA long. In another embodiment, the fragment is 13 or more AA. In another embodiment, the fragment is 14 or more AA. In another embodiment, the fragment is 15 or more AA. In another embodiment, the fragment is 17 or more AA. In another embodiment, the fragment is 18 or more AA. In another embodiment, the fragment is 19 or more AA. In another embodiment, the fragment is 22 or more AA. In another embodiment, the fragment is 8-12 AA. In another embodiment, the fragment is about 8-12 AA. In another embodiment, the fragment is 16-19 AA. In another embodiment, the fragment is about 16-19 AA. In another embodiment, the fragment 10-25 AA. In another embodiment, the fragment is about 10-25 AA. In another embodiment, the fragment has any other length. Each possibility represents a separate embodiment of the present invention.

In one embodiment, the present invention provides a composition comprising:
(a) a combination of at least seven isolated peptides consisting of:

(SEQ ID NO: 124)
YMFPNAPYL,
(SEQ ID NO: 1)
RSDELVREIFINMHQRNMTKL,
(SEQ ID NO: 2)
PGCNKRYFKLSHLQMEISRKHTG,
(SEQ ID NO: 125)
SGQAYMFPNAPYLPSCLES,
(SEQ ID NO: 21)
NLMNLGATL,
(SEQ ID NO: 26)
WNLMNLGATLKGVAA,
and
(SEQ ID NO: 205)
WNYMNLGATLKGVAA;

(b) a nucleic acid encoding the combination of at least seven isolated peptides of (a); or
(c) an immune cell comprising a nucleic acid encoding the combination of at least seven peptides of (a), and/or comprising or presenting the at least seven peptides of (a); or
(d) cytotoxic T cells (CTLs) induced by the combination of the at least seven isolated peptides of (a); or
(e) a combination of two, three, or all four from among (a), (b), (c), and (d).

In one embodiment, the composition comprises the combination of WT1 peptides including each of YMFPNAPYL (SEQ ID NO: 124; also referred to as WT1-A1), RSDELVRHHNMHQRNMTKL (SEQ ID NO: 1; also referred to as WT1-427 long), PGCNKRYFKLSHLQMHSRKHTG (SEQ ID NO: 2; also referred to as WT1-331 long), SGQAYMFPNAPYLPSCLES (SEQ ID NO: 125; also referred to as WT1-122A1 long), NLMNLGATL (SEQ ID NO: 21; also referred to as NLM short), WNLMNLGATLKGVAA (SEQ ID NO: 26; also referred to as WNLM or NLM long), and WNYMNLGATLKGVAA (SEQ ID NO: 205; also referred to as WNYM or NYM long). Optionally, the composition further comprises at least 1 additional WT1 peptide. In certain embodiments, a composition comprising at least 2 different isolated peptides of the present invention is provided. In certain embodiments, a composition comprising at least 3 or at least 4 different isolated peptides of the present invention is provided. Each possibility represents a separate embodiment of the present invention. In certain embodiments, the composition of the present invention is a vaccine.

In another embodiment, each peptide of the methods and compositions of the present invention independently binds an HLA class II molecule with significant affinity, while each peptide derived from the original peptide independently binds an HLA class I molecule with significant affinity, and pertains independently to each peptide comprising the combination.

In another embodiment, “affinity” refers to the concentration of peptide necessary for inhibiting binding of a standard peptide to the indicated MHC molecule by 50%. In another embodiment, “high affinity” refers to an affinity is such that a concentration of about 500 nanomolar (nM) or less of the peptide is required for 50% inhibition of binding of a standard peptide. In another embodiment, a concentration of about 400 nM or less of the peptide is required. In another embodiment, the binding affinity is 300 nM. In another embodiment, the binding affinity is 200 nM. In another embodiment, the binding affinity is 150 nM. In another embodiment, the binding affinity is 100 nM. In another embodiment, the binding affinity is 80 nM. In another embodiment, the binding affinity is 60 nM. In another embodiment, the binding affinity is 40 nM. In another embodiment, the binding affinity is 30 nM. In another embodiment, the binding affinity is 20 nM. In another embodiment, the binding affinity is 15 nM. In another embodiment, the binding affinity is 10 nM. In another embodiment, the binding affinity is 8 nM. In another embodiment, the binding affinity is 6 nM. In another embodiment, the binding affinity is 4 nM. In another embodiment, the binding affinity is 3 nM. In another embodiment, the binding affinity is 2 nM. In another embodiment, the binding affinity is 1.5 nM. In another embodiment, the binding affinity is 1 nM. In another embodiment, the binding affinity is 0.8 nM. In another embodiment, the binding affinity is 0.6 nM. In another embodiment, the binding affinity is 0.5 nM. In another embodiment, the binding affinity is 0.4 nM. In another embodiment, the binding affinity is 0.3 nM. In another embodiment, the binding affinity is less than 0.3 nM.

In another embodiment, “affinity” refers to a measure of binding strength to the MHC molecule. In another embodiment, affinity is measured using a method known in the art to measure competitive binding affinities. In another embodiment, affinity is measured using a method known in the art to measure relative binding affinities. In another embodiment, the method is a competitive binding assay. In another embodiment, the method is radioimmunoassay or RIA. In another embodiment, the method is BiaCore analyses. In another embodiment, the method is any other method known in the art. In another embodiment, the method yields an IC50 in relation to an IC50 of a reference peptide of known affinity.

Each type of affinity and method of measuring affinity represents a separate embodiment of the present invention.

In another embodiment, “high affinity” refers to an IC50 of 0.5-100 nM. In another embodiment, the IC50 is 1-100 nM. In another embodiment, the IC50 is 1.5-200 nM. In another embodiment, the IC50 is 2-100 nM. In another embodiment, the IC50 is 3-100 nM. In another embodiment, the IC50 is 4-100 nM. In another embodiment, the IC50 is 6-100 nM. In another embodiment, the IC50 is 10-100 nM. In another embodiment, the IC50 is 30-100 nM. In another embodiment, the IC50 is 3-80 nM. In another embodiment, the IC50 is 4-60 nM. In another embodiment, the IC50 is 5-50 nM. In another embodiment, the IC50 is 6-50 nM. In another embodiment, the IC50 is 8-50 nM. In another embodiment, the IC50 is 10-50 nM. In another embodiment, the IC50 is 20-50 nM. In another embodiment, the IC50 is 6-40 nM. In another embodiment, the IC50 is 8-30 nM. In another embodiment, the IC50 is 10-25 nM. In another embodiment, the IC50 is 15-25 nM. Each affinity and range of affinities represents a separate embodiment of the present invention.

In another embodiment, “medium affinity” refers to an IC50 of 100-500 nM. In another embodiment, the IC50 is 100-300 nM. In another embodiment, the IC50 is 100-200 nM. In another embodiment, the IC50 is 50-100 nM. In another embodiment, the IC50 is 50-80 nM. In another embodiment, the IC50 is 50-60 nM. Each affinity and range of affinities represents a separate embodiment of the present invention.

“Significant affinity” refers, in another embodiment, to sufficient affinity to mediate recognition of a target cell by a T cell carrying a T cell receptor (TCR) that recognizes the WIC molecule-peptide complex. In another embodiment, the term refers to sufficient affinity to mediate recognition of a cancer cell by a T cell carrying a TCR that recognizes the WIC molecule-peptide complex. In another embodiment, the term refers to sufficient affinity to mediate activation of a naive T cell by a dendritic cell presenting the peptide. In another embodiment, the term refers to sufficient affinity to mediate activation of a naive T cell by an APC presenting the peptide. In another embodiment, the term refers to sufficient affinity to mediate re-activation of a memory T cell by a dendritic cell presenting the peptide. In another embodiment, the term refers to sufficient affinity to mediate re-activation of a memory T cell by an APC presenting the peptide. In another embodiment, the term refers to sufficient affinity to mediate re-activation of a memory T cell by a somatic cell presenting the peptide. Each possibility represents a separate embodiment of the present invention.

“Measurable affinity” refers, in another embodiment, to sufficient affinity to be measurable by an immunological assay. In another embodiment, the immunological assay is any assay enumerated herein. Each possibility represents a separate embodiment of the present invention.

In another embodiment, a peptide of methods and compositions of the present invention binds to a superfamily of HLA molecules. Superfamilies of HLA molecules share very similar or identical binding motifs. In another embodiment, the superfamily is a HLA class I superfamily. In another embodiment, the superfamily is a HLA class II superfamily. Each possibility represents a separate embodiment of the present invention.

The terms “HLA-binding peptide,” “HLA class I molecule-binding peptide,” and “HLA class II molecule-binding peptide” refer, in another embodiment, to a peptide that binds an HLA molecule with measurable affinity. In another embodiment, the terms refer to a peptide that binds an HLA molecule with high affinity. In another embodiment, the terms refer to a peptide that binds an HLA molecule with sufficient affinity to activate a T cell precursor. In another embodiment, the terms refer to a peptide that binds an HLA molecule with sufficient affinity to mediate recognition by a T cell. The HLA molecule is, in other embodiments, any of the HLA molecules enumerated herein. Each possibility represents a separate embodiment of the present invention.

In another embodiment, a peptide of methods and compositions of the present invention is heteroclitic. “Heteroclitic” refers, in another embodiment, to a peptide that generates an immune response that recognizes the original peptide from which the heteroclitic peptide was derived (e.g. the peptide not containing the anchor residue mutations). In another embodiment, “original peptide” refers to a fragment of WT1 protein. For example, a peptide termed “WT1 122A1,” having the sequence SGQAYMFPNAPYLPSCLES (SEQ ID NO:124), was generated from the wild-type WT1 peptide SGQARMFPNAPYLPSCLES (SEQ ID NO:5) by mutation of residue 5 to arginine. The heteroclitic mutation introduced the CD8⁺ WT1 peptide RMFPNAPYL (SEQ ID NO:7) peptide generated YMFPNAPYL (SEQ ID NO:124), the WT1A1 peptide. In another embodiment, “heteroclitic” refers to a peptide that generates an immune response that recognizes the original peptide from which the heteroclitic peptide was derived, wherein the immune response generated by vaccination with the heteroclitic peptide is greater than the immune response generated by vaccination with the original peptide. In another embodiment, a “heteroclitic” immune response refers to an immune response that recognizes the original peptide from which the improved peptide was derived (e.g. the peptide not containing the anchor residue mutations). In another embodiment, a “heteroclitic” immune response refers to an immune response that recognizes the original peptide from which the heteroclitic peptide was derived, wherein the immune response generated by vaccination with the heteroclitic peptide is greater than the immune response generated by vaccination with the original peptide. In another embodiment, the magnitude of the immune response generated by vaccination with the heteroclitic peptide is greater than the immune response substantially equal to the response to vaccination with the original peptide. In another embodiment, the magnitude of the immune response generated by vaccination with the heteroclitic peptide is greater than the immune response less than the response to vaccination with the original peptide. Each possibility represents a separate embodiment of the present invention.

In another embodiment, a heteroclitic peptide of the present invention induces an immune response that is increased at least 2-fold relative to the WT1 peptide from which the heteroclitic peptide was derived (“native peptide”). In another embodiment, the increase is 3-fold relative to the native peptide. In another embodiment, the increase is 5-fold relative to the native peptide. In another embodiment, the increase is 7-fold relative to the native peptide. In another embodiment, the increase is 10-fold relative to the native peptide. In another embodiment, the increase is 15-fold relative to the native peptide. In another embodiment, the increase is 20-fold relative to the native peptide. In another embodiment, the increase is 30-fold relative to the native peptide. In another embodiment, the increase is 50-fold relative to the native peptide. In another embodiment, the increase is 100-fold relative to the native peptide. In another embodiment, the increase is 150-fold relative to the native peptide. In another embodiment, the increase is 200-fold relative to the native peptide. In another embodiment, the increase is 300-fold relative to the native peptide. In another embodiment, the increase is 500-fold relative to the native peptide. In another embodiment, the increase is 1000-fold relative to the native peptide. In another embodiment, the increase is more than 1000-fold relative to the native peptide. Each possibility represents a separate embodiment of the present invention and pertains independently to each heteroclitic, and native peptide derived therefrom, in the combination.

In another embodiment, a heteroclitic peptide of the present invention is an HLA class I heteroclitic peptide. In another embodiment, a heteroclitic peptide of the present invention is an HLA class II heteroclitic peptide. In another embodiment, a heteroclitic class II peptide of the present invention is mutated in a class II binding residue. In another embodiment, a heteroclitic class II peptide of the present invention is identified and tested in a manner analogous to identification and testing of HLA class I heteroclitic peptides, as exemplified herein. Each possibility represents a separate embodiment of the present invention.

“Anchor motifs” or “anchor residues” refers, in another embodiment, to one or a set of preferred residues at particular positions in an HLA-binding sequence. For example, residues at positions 1, 2, 3, 6, and 9 are used as anchor residues. In another embodiment, the HLA-binding sequence is an HLA class II-binding sequence. In another embodiment, the HLA-binding sequence is an HLA class I-binding sequence. In another embodiment, the positions corresponding to the anchor motifs are those that play a significant role in binding the HLA molecule. In another embodiment, the anchor residue is a primary anchor motif. In another embodiment, the anchor residue is a secondary anchor motif. Each possibility represents a separate embodiment of the present invention.

In another embodiment, “anchor residues” are residues in positions 1, 3, 6, and 9 of the HLA class I binding motif. In another embodiment, the term refers to positions 1, 2, 6, and 9 of the HLA class I binding motif. In another embodiment, the term refers to positions 1, 6, and 9 of the HLA class I binding motif. In another embodiment, the term refers to positions 1, 2, and 9 of the HLA class I binding motif. In another embodiment, the term refers to positions 1, 3, and 9 of the HLA class I binding motif. In another embodiment, the term refers to positions 2 and 9 of the HLA class I binding motif. In another embodiment, the term refers to positions 6 and 9 of the HLA class I binding motif. Each possibility represents a separate embodiment of the present invention.

Methods for identifying MHC class II epitopes are well known in the art. In another embodiment, the MHC class II epitope is predicted using TEPITOPE (Meister G E, Roberts C G et al, Vaccine 1995 13: 581-91). In another embodiment, the MHC class II epitope is identified using EpiMatrix (De Groot A S, Jesdale B M et al, AIDS Res Hum Retroviruses 1997 13: 529-31). In another embodiment, the MHC class II epitope is identified using the Predict Method (Yu K, Petrovsky N et al, Mol Med. 2002 8: 137-48). In another embodiment, the MHC class II epitope is identified using the SYFPEITHI epitope prediction algorithm. SYFPEITHI is a database comprising more than 4500 peptide sequences known to bind class I and class II MHC molecules. SYFPEITHI provides a score based on the presence of certain amino acids in certain positions along the MHC-binding groove. Ideal amino acid anchors are valued at 10 points, unusual anchors are worth 6-8 points, auxiliary anchors are worth 4-6 points, preferred residues are worth 1-4 points; negative amino acid effect on the binding score between −1 and −3. The maximum score for HLA-A*0201 is 36.

In another embodiment, the MHC class II epitope is identified using Rankpep. Rankpep uses position specific scoring matrices (PSSMs) or profiles from sets of aligned peptides known to bind to a given MHC molecule as the predictor of MHC-peptide binding. Rankpep includes information on the score of the peptide and the % optimum or percentile score of the predicted peptide relative to that of a consensus sequence that yields the maximum score, with the selected profile. Rankpep includes a selection of 102 and 80 PSSMs for the prediction of peptide binding to MHC I and MHC II molecules, respectively. Several PSSMs for the prediction of peptide binders of different sizes are usually available for each MHC I molecule.

In another embodiment, the MHC class II epitope is identified using SVMHC (Donnes P, Elofsson A. Prediction of MHC class I binding peptides, using SVMHC. BMC Bioinformatics. 2002 Sep. 11; 3:25). In another embodiment, the MHC class II epitope is identified using any other method known in the art. The above methods are utilized, in another embodiment, to identify MHC class II binding will be perturbed by introduction of an MHC class I anchor residue mutation into the WT1 sequence. Each possibility represents a separate embodiment of the present invention.

Methods for identifying MHC class I epitopes are well known in the art. In another embodiment, the MHC class I epitope is predicted using BIMAS software. The BIMAS score is based on the calculation of the theoretical half-life of the MHC-I/β₂-microglobulin/peptide complex, which is a measure of peptide-binding affinity. The program uses information about HLA-I peptides of 8-10 amino acids in length. The higher the binding affinity of a peptide to the MHC, the higher the likelihood that this peptide represents an epitope. The BIMAS algorithm assumes that each amino acid in the peptide contributes independently to binding to the class I molecule. Dominant anchor residues, which are critical for binding, have coefficients in the tables that are significantly higher than 1. Unfavorable amino acids have positive coefficients that are less than 1. If an amino acid is not known to make either a favorable or unfavorable contribution to binding, then is assigned the value 1. All the values assigned to the amino acids are multiplied and the resulting running score is multiplied by a constant to yield an estimate of half-time of dissociation.

In another embodiment, the MHC class I epitope is identified using SYFPEITHI. In another embodiment, the MHC class I epitope is identified using SVMHC (Donnes P, Elofsson A. Prediction of MHC class I binding peptides, using SVMHC. BMC Bioinformatics. 2002 Sep. 11; 3:25). In another embodiment, the MHC class I epitope is identified using NetMHC-2.0 (Sensitive quantitative predictions of peptide-MHC binding by a ‘Query by Committee’ artificial neural network approach. Buus S, Lauemoller S L, Worning P, Kesmir C, Frimurer T, Corbet S, Fomsgaard A, Hilden J, Holm A, Brunak S. Tissue Antigens., 62:378-84, 2003). In another embodiment, the MHC class I epitope is identified using any other method known in the art. The above methods are utilized, in another embodiment, to identify MHC class I epitopes that can be created by introduction of an anchor residue mutation into the WT1 sequence. Each possibility represents a separate embodiment of the present invention.

In another embodiment, the mutation that enhances MHC binding is in the residue at position 1 of the HLA class I binding motif. In another embodiment, the residue is changed to tyrosine. In another embodiment, the residue is changed to glycine. In another embodiment, the residue is changed to threonine. In another embodiment, the residue is changed to phenylalanine. In another embodiment, the residue is changed to any other residue known in the art. In another embodiment, a substitution in position 1 (e.g. to tyrosine) stabilizes the binding of the position 2 anchor residue.

In another embodiment, the mutation is in position 2 of the HLA class I binding motif. In another embodiment, the residue is changed to leucine. In another embodiment, the residue is changed to valine. In another embodiment, the residue is changed to isoleucine. In another embodiment, the residue is changed to methionine. In another embodiment, the residue is changed to any other residue known in the art.

In another embodiment, the mutation is in position 6 of the HLA class I binding motif. In another embodiment, the residue is changed to valine. In another embodiment, the residue is changed to cysteine. In another embodiment, the residue is changed to glutamine. In another embodiment, the residue is changed to histidine. In another embodiment, the residue is changed to any other residue known in the art.

In another embodiment, the mutation is in position 9 of the HLA class I binding motif. In another embodiment, the mutation changes the residue at the C-terminal position thereof. In another embodiment, the residue is changed to valine. In another embodiment, the residue is changed to threonine. In another embodiment, the residue is changed to isoleucine. In another embodiment, the residue is changed to leucine. In another embodiment, the residue is changed to alanine. In another embodiment, the residue is changed to cysteine. In another embodiment, the residue is changed to any other residue known in the art.

In another embodiment, the point mutation is in a primary anchor residue. In another embodiment, the HLA class I primary anchor residues are positions 2 and 9. In another embodiment, the point mutation is in a secondary anchor residue. In another embodiment, the HLA class I secondary anchor residues are positions 1 and 8. In another embodiment, the HLA class I secondary anchor residues are positions 1, 3, 6, 7, and 8. In another embodiment, the point mutation is in a position selected from positions 4, 5, and 8. Each possibility represents a separate embodiment of the present invention.

In another embodiment, the point mutation is in 1 or more residues in positions selected from positions 1, 2, 8, and 9 of the HLA class I binding motif. In another embodiment, the point mutation is in 1 or more residues in positions selected from positions 1, 3, 6, and 9. In another embodiment, the point mutation is in 1 or more residues in positions selected from positions 1, 2, 6, and 9. In another embodiment, the point mutation is in 1 or more residues in positions selected from positions 1, 6, and 9. In another embodiment, the point mutation is in 1 or more residues in positions selected from positions 1, 2, and 9. In another embodiment, the point mutation is in 1 or more residues in positions selected from positions 1, 3, and 9. In another embodiment, the point mutation is in 1 or more residues in positions selected from positions 2 and 9. In another embodiment, the point mutation is in 1 or more residues in positions selected from positions 6 and 9. Each possibility represents a separate embodiment of the present invention. In another embodiment, the mutation is in the 4 position of the HLA class I binding motif. In another embodiment, the mutation is in the 5 position of the HLA class I binding motif. In another embodiment, the mutation is in the 7 position of the HLA class I binding motif. In another embodiment, the mutation is in the 8 position of the HLA class I binding motif. Each possibility represents a separate embodiment of the present invention.

Each of the above anchor residues and substitutions represents a separate embodiment of the present invention.

In another embodiment, the HLA class II binding site in a peptide of the present invention is created or improved by mutation of an HLA class II motif anchor residue. In another embodiment, the anchor residue that is modified is in the P1 position. In another embodiment, the anchor residue is at the P2 position. In another embodiment, the anchor residue is at the P6 position. In another embodiment, the anchor residue is at the P9 position. In another embodiment, the anchor residue is selected from the P1, P2, P6, and P9 positions. In another embodiment, the anchor residue is at the P3 position. In another embodiment, the anchor residue is at the P4 position. In another embodiment, the anchor residue is at the P5 position. In another embodiment, the anchor residue is at the P6 position. In another embodiment, the anchor residue is at the P8 position. In another embodiment, the anchor residue is at the P10 position. In another embodiment, the anchor residue is at the P11 position. In another embodiment, the anchor residue is at the P12 position. In another embodiment, the anchor residue is at the P13 position. In another embodiment, the anchor residue is at any other anchor residue of an HLA class II molecule that is known in the art. In another embodiment, residues other than P1, P2, P6, and P9 serve as secondary anchor residues; therefore, mutating them can improve HLA class II binding. In another embodiment, any combination of the above residues is mutated. Each possibility represents a separate embodiment of the present invention.

In another embodiment, the present invention provides a method of inducing an anti-WT1-expressing cancer immune response in a subject, the method comprising the step of administering an immunotherapy composition disclosed herein to the subject, optionally with at least one checkpoint inhibitor, thereby inducing an anti-WT1-expressing cancer immune response in a subject.

In another embodiment, the present invention provides a method of treating a subject with a WT1-expressing cancer, the method comprising the step of administering to the subject an immunotherapy composition disclosed herein to the subject, optionally with at least one checkpoint inhibitor, thereby treating a subject with a WT1-expressing cancer.

In another embodiment, the present invention provides a method of reducing an incidence of a WT1-expressing cancer, or its relapse, in a subject, the method comprising the step of administering to the subject an immunotherapy composition disclosed herein, optionally with at least one checkpoint inhibitor, thereby reducing an incidence of a WT1-expressing cancer, or its relapse, in a subject.

The terms “homology,” “homologous,” etc., when in reference to any protein or peptide, refer, in another embodiment, to a percentage of AA residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, the term “homology,” when in reference to any nucleic acid sequence similarly indicates a percentage of nucleotides in a candidate sequence that are identical with the nucleotides of a corresponding native nucleic acid sequence.

Homology is, in another embodiment, determined by computer algorithm for sequence alignment, by methods well described in the art. In other embodiments, computer algorithm analysis of nucleic acid sequence homology includes the utilization of any number of software packages available, such as, for example, the BLAST, DOMAIN, BEAUTY (BLAST Enhanced Alignment Utility), GENPEPT and TREMBL packages.

The percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm in a sequence analysis software. Protein analysis software matches similar sequences using measures of similarity assigned to various substitutions, deletions and other modifications, including conservative amino acid substitutions.

The percent identity between two amino acid sequences can be determined, e.g., using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Polypeptide sequences can also be compared using FASTA, applying default or recommended parameters. A program in GCG Version 6.1., FASTA (e.g., FASTA2 and FASTA3) provides alignments and percent sequence identity of the regions of the best overlap between the query and search sequences (Pearson, Methods Enzymol. 1990; 183:63-98; Pearson, Methods Mol. Biol. 2000; 132:185-219). The percent identity between two amino acid sequences can also be determined using the algorithm of E. Meyers and W. Miller (Comput. Appl. Biosci., 1988; 11-17) which has been incorporated into the ALIGN program (version 2.0), using a PAM120 weight residue table, a gap length penalty of 12 and a gap penalty of 4.

Another algorithm for comparing a sequence to other sequences contained in a database is the computer program BLAST, especially blastp, using default parameters. See, e.g., Altschul et al., J. Mol. Biol. 1990; 215:403-410; Altschul et al., Nucleic Acids Res. 1997; 25:3389-402 (1997); each herein incorporated by reference. The protein sequences of the present invention can there be used as a “query sequence” to perform a search against public databases to, for example, identify related sequences. Such searches can be performed using the XBLAST program (version 2.0) of Altschul, et al. 1990 (supra). BLAST protein searches can be performed with the XBLAST program, score=50, wordlength=3 to obtain amino acid sequences homologous to WT1 peptides of the invention. To obtain gapped alignments for comparison purposes, Gapped BLAST can be utilized as described in Altschul et al., 1997 (supra). When utilizing BLAST and Gapped BLAST programs, the default parameters of the respective programs (e.g., XBLAST and NBLAST) can be used.

In another embodiment, “homology” with respect to a homologous sequence refers to percent identity to a sequence disclosed herein of greater than 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. Each possibility represents a separate embodiment of the present invention.

In another embodiment, the present invention provides a composition comprising one or more WT1 delivery agents to deliver the combination of at least seven WT1 peptides, or CTLs induced by the at least seven WT1 peptides, and at least one checkpoint inhibitor. In another embodiment, the composition further comprises a pharmaceutically acceptable carrier. In another embodiment, the composition further comprises an adjuvant. In another embodiment, the composition comprises 2 or more peptides of the present invention. In another embodiment, the composition further comprises any of the additives, compounds, or excipients set forth herein below. In another embodiment, the adjuvant is an alum salt or other mineral adjuvant, bacterial product or bacteria-derived adjuvant, tensoactive agent (e.g., saponin), o/w or w/o emulsion, liposome adjuvant, cytokine (e.g., IL-2, GM-CSF, IL-12, and IFN-gamma), or alpha-galactosylceramide analog. In another embodiment, the adjuvant is QS21, Freund's complete or incomplete adjuvant, aluminum phosphate, aluminum hydroxide, BCG or alum. In other embodiments, the carrier is any carrier enumerated herein. In other embodiments, the adjuvant is any adjuvant enumerated herein. Each possibility represents a separate embodiment of the present invention.

In another embodiment, this invention provides a vaccine comprising one or more WT1 delivery agents to deliver the combination of at least seven WT1 peptides, or CTLs, and at least one checkpoint inhibitor. In another embodiment, the vaccine further comprises a carrier. In another embodiment, the vaccine further comprises an adjuvant. In another embodiment, the vaccine further comprises a combination of a carrier and an adjuvant. In another embodiment, the vaccine further comprises an APC. In another embodiment, the vaccine further comprises a combination of an APC and a carrier or an adjuvant. In another embodiment, the vaccine is a cell-based composition. Each possibility represents a separate embodiment of the present invention.

In another embodiment, this invention provides an immunogenic composition comprising a peptide of the present invention and at least one checkpoint inhibitor. In another embodiment, the immunogenic composition further comprises a carrier. In another embodiment, the immunogenic composition further comprises an adjuvant. In another embodiment, the immunogenic composition further comprises a combination of a carrier and an adjuvant. Each possibility represents a separate embodiment of the present invention.

In another embodiment, the term “vaccine” refers to a material or composition that, when introduced into a subject, provides a prophylactic or therapeutic response for a particular disease, condition, or symptom of same. In another embodiment, this invention comprises peptide-based vaccines, wherein the peptide comprises any embodiment listed herein, optionally further including immunomodulating compounds such as cytokines, adjuvants, etc.

In other embodiments, a composition or vaccine of methods and compositions of the present invention further comprises an adjuvant. In another embodiment, the adjuvant is Montanide ISA 51. Montanide ISA 51 contains a natural metabolizable oil and a refined emulsifier. In another embodiment, the adjuvant is GM-CSF. In another embodiment, the adjuvant is keyhole limpet hemocyanin (KLH), which may be conjugated to the peptide antigen or may be administered together with the peptide. Recombinant GM-CSF is a human protein grown, in another embodiment, in a yeast (S. cerevisiae) vector. GM-CSF promotes clonal expansion and differentiation of hematopoietic progenitor cells, APC, and dendritic cells and T cells.

In another embodiment, the adjuvant is a cytokine. In another embodiment, the adjuvant is a growth factor. In another embodiment, the adjuvant is a cell population. In another embodiment, the adjuvant is QS21. In another embodiment, the adjuvant is Freund's incomplete adjuvant. In another embodiment, the adjuvant is aluminum phosphate. In another embodiment, the adjuvant is aluminum hydroxide. In another embodiment, the adjuvant is BCG. In another embodiment, the adjuvant is alum. In another embodiment, the adjuvant is an interleukin. In another embodiment, the adjuvant is a chemokine. In another embodiment, the adjuvant is any other type of adjuvant known in the art. In another embodiment, the WT1 vaccine comprises two of the above adjuvants. In another embodiment, the WT1 vaccine comprises more than two of the above adjuvants. Each possibility represents a separate embodiment of the present invention.

In another embodiment, the WT1 vaccine used in the methods of the present invention can be one or more nucleic acid molecules (DNA or RNA) encoding one or more WT1 peptides of the present invention. In the practice of this embodiment, a vaccine comprising nucleic acid molecules encoding the one or more WT1 peptides (a nucleic acid vaccine) is administered and one or more checkpoint inhibitors are administered to the patient. In all other embodiments of the invention, the nucleic acid vaccine can be used in place of the peptide vaccine. The nucleic acid may be introduced alone, as part of a viral carrier, or inside of a cell, possibly as a plasmid or integrated into the cell's nucleic acid. The cell carrier may be the patient's cells, removed from the patient, or a cell from a donor, or a cell line. The cell may be an antigen presenting cell such as a dendritic cell or monocyte/macrophage lineage cell. The cellular vector is selected from the group consisting of a cell, such as autologous cell, allogeneic cell, cell line, dendritic cell or antigen presenting cell, or fusion of any of the above cells into a hybrid cell.

The WT1 peptide or the nucleic acid encoding it, or its carrier in any of the forms herein described may be exposed to the CTL's ex vivo or in vivo. If in vitro or ex vivo, the cells may be grown or expanded and then introduced into the patient.

As used interchangeably herein, the terms “nucleic acid”, “nucleic acid molecule”, “oligonucleotide”, and “polynucleotide” include RNA, DNA, or RNA/DNA hybrid sequences of more than one nucleotide in either single chain or duplex form. The terms encompass “modified nucleotides” which comprise at least one modification, including by way of example and not limitation: (a) an alternative linking group, (b) an analogous form of purine, (c) an analogous form of pyrimidine, or (d) an analogous sugar. For examples of analogous linking groups, purines, pyrimidines, and sugars see for example PCT publication No. WO 95/04064. The nucleic acid sequences of the invention may be prepared by any known method, including synthetic, recombinant, ex vivo generation, or a combination thereof, as well as utilizing any purification methods known in the art. As used herein, the term “nucleic acid vaccine” is inclusive of DNA vaccines and RNA vaccines, and vaccines comprising a viral or non-viral vector.

In another embodiment, the uses of the present invention provides a vector comprising a nucleic acid molecule (DNA or RNA). In other embodiments, a composition or vaccine used in the practice of the present invention can comprise any of the embodiments of WT1 peptides of the present invention and combinations thereof. Each possibility represents a separate embodiment of the present invention.

In another embodiment, a vaccine for use in the practice of the present invention or a composition of the present invention comprises two peptides that are derived from the same WT1 fragment, each containing a different HLA class I heteroclitic peptide. In another embodiment, the two HLA class I heteroclitic peptides contain mutations in different HLA class I molecule anchor residues. In another embodiment, the two HLA class I heteroclitic peptides contain different mutations in the same anchor residue(s). Each possibility represents a separate embodiment of the present invention.

In another embodiment, the peptides in a composition used in the present invention bind to two distinct HLA class II molecules. In another embodiment, the peptides bind to three distinct HLA class II molecules. In another embodiment, the peptides bind to four distinct HLA class II molecules. In another embodiment, the peptides bind to five distinct HLA class II molecules. In another embodiment, the peptides bind to more than five distinct HLA class II molecules. In another embodiment, the peptides in the composition bind to the same HLA class II molecules.

In another embodiment, each of the peptides in a composition or method of use of the present invention binds to a set of HLA class II molecules. In another embodiment, each of the peptides binds to a distinct set of HLA class II molecules. In another embodiment, the peptides in the composition bind to the same set of HLA class II molecules. In another embodiment, two of the peptides bind to a distinct but overlapping set of HLA class II molecules. In another embodiment, two or more of the peptides bind to the same set of HLA class II molecules, while another of the peptides binds to a distinct set. In another embodiment, two or more of the peptides bind to an overlapping set of HLA class II molecules, while another of the peptides binds to a distinct set.

In another embodiment, the peptides for use in the practice of the invention or in a composition of the present invention bind to two distinct HLA class I molecules. In another embodiment, the peptides bind to three distinct HLA class I molecules. In another embodiment, the peptides bind to four distinct HLA class I molecules. In another embodiment, the peptides bind to five distinct HLA class I molecules. In another embodiment, the peptides bind to more than five distinct HLA class I molecules. In another embodiment, the peptides in the composition bind to the same HLA class I molecules.

In another embodiment, each of the peptides for use in the practice of the invention or in a composition of the present invention binds to a set of HLA class I molecules. In another embodiment, each of the peptides binds to a distinct set of HLA class I molecules. In another embodiment, the peptides in the composition bind to the same set of HLA class I molecules. In another embodiment, two of the peptides bind to a distinct but overlapping set of HLA class I molecules. In another embodiment, two or more of the peptides bind to the same set of HLA class I molecules, while another of the peptides binds to a distinct set. In another embodiment, two or more of the peptides bind to an overlapping set of HLA class I molecules, while another of the peptides binds to a distinct set.

In another embodiment, a “set of HLA class II molecules” or “set of HLA class I molecules” refers to the HLA molecules encoded by different alleles at a particular locus. In another embodiment, the term refers to HLA molecules with a particular binding specificity. In another embodiment, the term refers to HLA molecules with a particular peptide consensus sequence. In another embodiment, the term refers to a superfamily of HLA class II molecules. Each possibility represents a separate embodiment of the present invention.

Each of the above compositions and types of compositions represents a separate embodiment of the present invention.

Any embodiments described herein regarding peptides, nucleic acids, compositions, and vaccines of this invention may be employed in any of the methods of this invention. Each combination of peptide, nucleic acid, composition, or vaccine with a method represents a separate embodiment thereof.

In another embodiment, the present invention provides a method of treating a subject with a WT1-expressing cancer, the method comprising administering to the subject a WT1 immunotherapy composition as described herein and, optionally, a checkpoint inhibitor, thereby treating a subject with a WT1-expressing cancer. In another embodiment, the present invention provides a method of treating a subject with a WT1-expressing cancer, the method comprising administering to the subject a composition of the present invention comprising at least WT1 delivery agent for delivering the combination at least seven WT1 peptides, or CTLs induced by the at least seven peptides, and, optionally, at least one checkpoint inhibitor, thereby treating a subject with a WT1-expressing cancer. In another embodiment, the present invention provides a method of treating a subject with a WT1-expressing cancer, the method comprising administering to the subject an immunogenic composition such as a vaccine and, optionally, a checkpoint inhibitor, thereby treating a subject with a WT1-expressing cancer.

In another embodiment, the present invention provides a method of suppressing or halting the progression of a WT1-expressing cancer in a subject, the method comprising administering to the subject one or more WT1 delivery agents to deliver the combination of at least seven WT1 peptides, or CTLs induced by the at least seven WT1 peptides, and, optionally, at least one checkpoint inhibitor, thereby suppressing or halting the progression of a WT1-expressing cancer. In another embodiment, the present invention provides a method of suppressing or halting the progression of a WT1-expressing cancer in a subject, the method comprising administering to the subject a composition comprising the combination of at least seven WT1 peptides and, optionally, at least one checkpoint inhibitor, thereby suppressing or halting the progression of a WT1-expressing cancer. In another embodiment, the present invention provides a method of suppressing or halting the progression of a WT1-expressing cancer in a subject, the method comprising administering to the subject an immunogenic composition such as an immunotherapy composition of the present invention, comprising the combination of at least seven WT1 peptides and, optionally, at least one checkpoint inhibitor, thereby suppressing or halting the progression of a WT1-expressing cancer

In another embodiment, the present invention provides a method of reducing the incidence of a WT1-expressing cancer in a subject, the method comprising administering to the subject one or more WT1 delivery agents to deliver the combination of at least seven WT1 peptides, or CTLs induced by the at least seven peptides, and optionally, and at least one checkpoint inhibitor, thereby reducing the incidence of a WT1-expressing cancer in a subject. In another embodiment, the present invention provides a method of reducing the incidence of a WT1-expressing cancer in a subject, the method comprising administering to the subject a composition of the present invention comprising the one or more WT1 delivery agents to deliver the combination at least seven WT1 peptides, or CTLs induced by the at least seven WT1 peptides and, optionally, at least one checkpoint inhibitor, thereby reducing the incidence of a WT1-expressing cancer in a subject. In another embodiment, the present invention provides a method of reducing the incidence of a WT1-expressing cancer in a subject, the method comprising administering to the subject a composition of the present invention comprising the combination of at least seven WT1 peptides and, optionally, at least one checkpoint inhibitor, thereby reducing the incidence of a WT1-expressing cancer in a subject.

In another embodiment, the present invention provides a method of reducing the incidence of relapse of a WT1-expressing cancer in a subject, the method comprising administering to the subject a composition comprising one or more WT1 delivery agents to deliver the combination of at least seven WT1 peptides, or CTLs induced by the at least seven WT1 peptides and, optionally, at least one checkpoint inhibitor, thereby reducing the incidence of relapse of a WT1-expressing cancer in a subject. In another embodiment, the present invention provides a method of reducing the incidence of relapse of a WT1-expressing cancer in a subject, the method comprising administering to the subject a composition of the present invention comprising the combination of at least seven WT1 peptides and, optionally, at least one checkpoint inhibitor, thereby reducing the incidence of relapse of a WT1-expressing cancer in a subject.

In another embodiment, the present invention provides a method of overcoming a T cell tolerance of a subject to a WT1-expressing cancer, the method comprising administering to the subject one or more WT1 delivery agents for delivering the combination of at least seven WT1 peptides, or CTLs induced by the at least seven WT1 peptides and, optionally, at least one checkpoint inhibitor, thereby overcoming a T cell tolerance to a WT1-expressing cancer. In another embodiment, the present invention provides a method of overcoming a T cell tolerance of a subject to a WT1-expressing cancer, the method comprising administering to the subject a composition of the present invention comprising the combination of at least seven WT1 peptides and, optionally, at least one checkpoint inhibitor, thereby overcoming a T cell tolerance to a WT1-expressing cancer. In another embodiment, the present invention provides a method of overcoming a T cell tolerance of a subject to a WT1-expressing cancer, the method comprising administering to the subject an immunogenic composition such as an immunotherapy composition of the present invention comprising the combination of at least seven WT1 peptides and, optionally, at least one checkpoint inhibitor, thereby overcoming a T cell tolerance to a WT1-expressing cancer

In another embodiment, the present invention provides a method of treating a subject having a WT1-expressing cancer, comprising (a) inducing in a donor formation and proliferation of human cytotoxic T lymphocytes (CTL) that recognize a malignant cell of the cancer by a method of the present invention; and (b) infusing the human CTL into the subject, thereby treating a subject having a cancer. In one embodiment, the donor is administered the combination of at least seven WT1 peptides, and the CTL from said donor are infused into the subject and, optionally, the subject is administered a checkpoint inhibitor, thereby treating a subject having a cancer. In one embodiment, the donor is administered the combination of at least seven WT1 peptides and, optionally, at least one checkpoint inhibitor, and the CTL from said donor are infused into the subject and the subject, thereby treating a subject having a cancer. In one embodiment, the donor is administered the combination of at least seven WT1 peptides and, optionally, at least one checkpoint inhibitor, and the CTL from said donor are infused into the subject and, optionally, the subject is administered a checkpoint inhibitor, thereby treating a subject having a cancer.

In another embodiment, the present invention provides a method of treating a subject having a WT1-expressing cancer, comprising (a) inducing ex vivo formation and proliferation of human CTL that recognize a malignant cell of the cancer by a method of the present invention, wherein the human immune cells are obtained from a donor; and (b) infusing the human CTL into the subject, thereby treating a subject having a cancer. In one embodiment, a checkpoint inhibitor is included in the ex vivo step. In another embodiment a checkpoint inhibitor is administered to the subject. In another embodiment both the ex vivo step includes a checkpoint inhibitor, and the subject is also administered a checkpoint inhibitor.

Methods for ex vivo immunotherapy are well known in the art and are described, for example, in Davis I D et al (Blood dendritic cells generated with Flt3 ligand and CD40 ligand prime CD8+ T cells efficiently in cancer patients. J Immunother. 2006 September-October; 29(5):499-511) and Mitchell M S et al (The cytotoxic T cell response to peptide analogs of the HLA-A*0201-restricted MUC1 signal sequence epitope, M1.2. Cancer Immunol Immunother. 2006 Jul. 28). Each method represents a separate embodiment of the present invention.

In another embodiment, the present invention provides a method of inducing formation and proliferation of a WT1 protein-specific CTL, the method comprising contacting a lymphocyte population with an immunogenic composition such as an immunotherapy composition of the present invention, optionally, together with at least one checkpoint inhibitor, thereby inducing formation and proliferation of a WT1 protein-specific CTL. In another embodiment, the immunogenic composition comprises an antigen-presenting cell (APC) associated with a peptide of the present invention and a checkpoint inhibitor. In another embodiment, the present invention provides a method of inducing formation and proliferation of a WT1 protein-specific CTL, the method comprising contacting a lymphocyte population with a peptide or composition of the present invention, together with at least one checkpoint inhibitor, thereby inducing formation and proliferation of a WT1 protein-specific CTL. In another embodiment, the present invention provides a method of inducing formation and proliferation of a WT1 protein-specific CTL, the method comprising contacting a lymphocyte population with a vaccine of the present invention, together with at least one checkpoint inhibitor, thereby inducing formation and proliferation of a WT1 protein-specific CTL. In another embodiment, the CTL is specific for a WT1-expressing cell. In another embodiment, the target cell is a cell of a WT1-expressing cancer. Each possibility represents a separate embodiment of the present invention.

In another embodiment, the present invention provides a method of inducing in a subject formation and proliferation of a WT1 protein-specific CTL, the method comprising contacting the subject with an immunogenic composition such as an immunotherapy composition of the present invention, optionally, together with at least one checkpoint inhibitor, thereby inducing in a subject formation and proliferation of a WT1 protein-specific CTL. In another embodiment, the immunogenic composition comprises an APC associated with a mixture of the at least seven WT1 peptides of the present invention, which is administered together with at least one checkpoint inhibitor. In another embodiment, the present invention provides a method of inducing in a subject formation and proliferation of a WT1 protein-specific CTL, the method comprising contacting the subject with the combination of at least seven WT1 peptides together with at least one checkpoint inhibitor, or composition of the present invention, thereby inducing in a subject formation and proliferation of a WT1 protein-specific CTL. In another embodiment, the present invention provides a method of inducing in a subject formation and proliferation of a WT1 protein-specific CTL, the method comprising contacting the subject with a vaccine of the present invention, together with at least one checkpoint inhibitor, thereby inducing in a subject formation and proliferation of a WT1 protein-specific CTL. In another embodiment, the target cell is a cell of a WT1-expressing cancer. In another embodiment, the subject has the WT1-expressing cancer. In another embodiment, the CTL is specific for a WT1-expressing cell.

In another embodiment, this invention provides a method of generating a heteroclitic immune response in a subject, wherein the heteroclitic immune response is directed against a WT1-expressing cancer, the method comprising administering to the subject the combination of at least seven WT1 peptides, optionally, together with at least one checkpoint inhibitor, or composition of the present invention, thereby generating a heteroclitic immune response. In another embodiment, this invention provides a method of generating a heteroclitic immune response in a subject, wherein the heteroclitic immune response is directed against a WT1-expressing cancer, the method comprising administering to the subject an immunogenic composition such as a vaccine of the present invention, together with at least one checkpoint inhibitor, thereby generating a heteroclitic immune response. In another embodiment, this invention provides a method of generating a heteroclitic immune response in a subject, wherein the heteroclitic immune response is directed against a WT1-expressing cancer, the method comprising administering to the subject a vaccine of the present invention, together with at least one checkpoint inhibitor, thereby generating a heteroclitic immune response.

Each method represents a separate embodiment of the present invention.

In another embodiment, the WT1-expressing cancer is an acute myelogenous leukemia (AML). In another embodiment, the WT1-expressing cancer is a chronic myelogenous leukemia (CIVIL). In another embodiment, the WT1-expressing cancer is associated with a myelodysplastic syndrome (MDS). In another embodiment, the WT1-expressing cancer is an MDS. In another embodiment, the WT1-expressing cancer is a non-small cell lung cancer (NSCLC). In another embodiment, the WT1-expressing cancer is an esophageal squamous cell carcinoma. In another embodiment, the WT1-expressing cancer is an acute lymphoblastic leukemia (ALL). In another embodiment, the WT1-expressing cancer is a bone or soft tissue sarcoma. In another embodiment, the WT1-expressing cancer is a Wilms' tumor. In another embodiment, the WT1-expressing cancer is a leukemia. In another embodiment, the WT1-expressing cancer is a hematological cancer. In another embodiment, the WT1-expressing cancer is a lymphoma. In another embodiment, the WT1-expressing cancer is a desmoplastic small round cell tumor. In another embodiment, the WT1-expressing cancer is a mesothelioma. In another embodiment, the WT1-expressing cancer is a malignant mesothelioma. In another embodiment, the WT1-expressing cancer is a gastric cancer. In another embodiment, the WT1-expressing cancer is a colon cancer. In another embodiment, the WT1-expressing cancer is a lung cancer. In another embodiment, the WT1-expressing cancer is a breast cancer. In another embodiment, the WT1-expressing cancer is a germ cell tumor. In another embodiment, the WT1-expressing cancer is a malignant pleural mesothelioma. In another embodiment, the WT1-expressing cancer is multiple myeloma. In another embodiment, the WT1-expressing cancer is myeloid leukemia. In another embodiment, the WT1-expressing cancer is an astrocytic cancer. In another embodiment, the WT1-expressing cancer is a glioblastoma (e.g., glioblastoma multiforme). In another embodiment, the WT1-expressing cancer is a colorectal adenocarcinoma. In another embodiment, the WT1-expressing cancer is an ovarian cancer (e.g., serous, epithelial, or endometrial). In another embodiment, the WT1-expressing cancer is breast cancer. In another embodiment, the WT1-expressing cancer is melanoma. In another embodiment, the WT1-expressing cancer is head and neck squamous cell carcinoma. In another embodiment, the WT1-expressing cancer is pancreatic ductal cell carcinoma. In another embodiment, the WT1-expressing cancer is a neuroblastoma. In another embodiment, the WT1-expressing cancer is a uterine cancer. In another embodiment, the WT1-expressing cancer is a thyroid cancer. In another embodiment, the WT1-expressing cancer is a hepatocellular carcinoma. In another embodiment, the WT1-expressing cancer is a thyroid cancer. In another embodiment, the WT1-expressing cancer is a liver cancer. In another embodiment, the WT1-expressing cancer is a renal cancer (e.g., renal cell carcinoma). In another embodiment, the WT1-expressing cancer is a Kaposi's sarcoma. In another embodiment, the WT1-expressing cancer is a sarcoma. In another embodiment, the WT1-expressing cancer is any other carcinoma or sarcoma.

In another embodiment, the WT1-expressing cancer is a solid tumor. In another embodiment, the solid tumor is associated with a WT1-expressing cancer. In another embodiment, the solid tumor is associated with a myelodysplastic syndrome (MDS). In another embodiment, the solid tumor is associated with a non-small cell lung cancer (NSCLC). In another embodiment, the solid tumor is associated with a lung cancer. In another embodiment, the solid tumor is associated with a breast cancer. In another embodiment, the solid tumor is associated with a colorectal cancer. In another embodiment, the solid tumor is associated with a prostate cancer. In another embodiment, the solid tumor is associated with an ovarian cancer. In another embodiment, the solid tumor is associated with a renal cancer. In another embodiment, the solid tumor is associated with a pancreatic cancer. In another embodiment, the solid tumor is associated with a brain cancer. In another embodiment, the solid tumor is associated with a gastrointestinal cancer. In another embodiment, the solid tumor is associated with a skin cancer. In another embodiment, the solid tumor is associated with a melanoma.

In another embodiment, a cancer or tumor treated by a method of the present invention is suspected to express WT1. In another embodiment, WT1 expression has not been verified by testing of the actual tumor sample. In another embodiment, the cancer or tumor is of a type known to express WT1 in many cases. In another embodiment, the type expresses WT1 in the majority of cases.

Each type of WT1-expressing cancer or tumor, and cancer or tumor suspected to express WT1, represents a separate embodiment of the present invention.

A non-exhaustive list of cancer types that may be treated using the compositions and methods of the invention is provided in Table 2.

TABLE 2
Examples of Cancer Types

| | |
|---|---|
| Acute Lymphoblastic Leukemia, Adult | Hairy Cell Leukemia |
| Acute Lymphoblastic Leukemia, | Head and Neck Cancer |
| Childhood | Hepatocellular (Liver) Cancer, Adult |
| Acute Myeloid Leukemia, Adult | (Primary) |
| Acute Myeloid Leukemia, Childhood | Hepatocellular (Liver) Cancer, Childhood |
| Adrenocortical Carcinoma | (Primary) |
| Adrenocortical Carcinoma, Childhood | Hodgkin’s Lymphoma, Adult |
| AIDS-Related Cancers | Hodgkin’s Lymphoma, Childhood |
| AIDS-Related Lymphoma | Hodgkin’s Lymphoma During Pregnancy |
| Anal Cancer | Hypopharyngeal Cancer |
| Astrocytoma, Childhood Cerebellar | Hypothalamic and Visual Pathway Glioma, |
| Astrocytoma, Childhood Cerebral | Childhood |
| Basal Cell Carcinoma | Intraocular Melanoma |
| Bile Duct Cancer, Extrahepatic | Islet Cell Carcinoma (Endocrine Pancreas) |
| Bladder Cancer | Kaposi’s Sarcoma |
| Bladder Cancer, Childhood | Kidney (Renal Cell) Cancer |
| Bone Cancer, Osteosarcoma/Malignant | Kidney Cancer, Childhood |
| Fibrous Histiocytoma | Laryngeal Cancer |
| Brain Stem Glioma, Childhood | Laryngeal Cancer, Childhood |
| Brain Tumor, Adult | Leukemia, Acute Lymphoblastic, Adult |
| Brain Tumor, Brain Stem Glioma, | Leukemia, Acute Lymphoblastic, Childhood |
| Childhood | Leukemia, Acute Myeloid, Adult |
| Brain Tumor, Cerebellar Astrocytoma, | Leukemia, Acute Myeloid, Childhood |
| Childhood | Leukemia, Chronic Lymphocytic |
| Brain Tumor, Cerebral | Leukemia, Chronic Myelogenous |
| Astrocytoma/Malignant Glioma, | Leukemia, Hairy Cell |
| Childhood | Lip and Oral Cavity Cancer |
| Brain Tumor, Ependymoma, Childhood | Liver Cancer, Adult (Primary) |
| Brain Tumor, Medulloblastoma, | Liver Cancer, Childhood (Primary) |
| Childhood | Lung Cancer, Non-Small Cell |
| Brain Tumor, Supratentorial Primitive | Lung Cancer, Small Cell |
| Neuroectodermal Tumors, Childhood | Lymphoma, AIDS-Related |
| Brain Tumor, Visual Pathway and | Lymphoma, Burkitt’s |
| Hypothalamic Glioma, Childhood | Lymphoma, Cutaneous T-Cell, see Mycosis |
| Brain Tumor, Childhood | Fungoides and Sézary Syndrome |
| Breast Cancer | Lymphoma, Hodgkin’s, Adult |
| Breast Cancer, Childhood | Lymphoma, Hodgkin’s, Childhood |
| Breast Cancer, Male | Lymphoma, Hodgkin’s During Pregnancy |
| Bronchial Adenomas/Carcinoids, | Lymphoma, Non-Hodgkin’s, Adult |
| Childhood | Lymphoma, Non-Hodgkin’s, Childhood |
| Burkitt’s Lymphoma | Lymphoma, Non-Hodgkin’s During Pregnancy |
| Carcinoid Tumor, Childhood | Lymphoma, Primary Central Nervous System |
| Carcinoid Tumor, Gastrointestinal | Macroglobulinemia, Waldenström’s |
| Carcinoma of Unknown Primary | Malignant Fibrous Histiocytoma of |
| Central Nervous System Lymphoma, | Bone/Osteosarcoma |
| Primary | Medulloblastoma, Childhood |
| Cerebellar Astrocytoma, Childhood | Melanoma |
| Cerebral Astrocytoma/Malignant | Melanoma, Intraocular (Eye) |
| Glioma, Childhood | Merkel Cell Carcinoma |
| Cervical Cancer | Mesothelioma, Adult Malignant |
| Childhood Cancers | Mesothelioma, Childhood |
| Chronic Lymphocytic Leukemia | Metastatic Squamous Neck Cancer with |
| Chronic Myelogenous Leukemia | Occult Primary |
| Chronic Myeloproliferative Disorders | Multiple Endocrine Neoplasia Syndrome, |
| Colon Cancer | Childhood |
| Colorectal Cancer, Childhood | Multiple Myeloma/Plasma Cell Neoplasm |
| Cutaneous T-Cell Lymphoma, see | Mycosis Fungoides |
| Mycosis Fungoides and Sezary | Myelodysplastic Syndromes |
| Syndrome | Myelodysplastic/Myeloproliferative Diseases |
| Endometrial Cancer | Myelogenous Leukemia, Chronic |
| Ependymoma, Childhood | Myeloid Leukemia, Adult Acute |
| Esophageal Cancer | Myeloid Leukemia, Childhood Acute |
| Esophageal Cancer, Childhood | Myeloma, Multiple |
| Ewing’s Family of Tumors | Myeloproliferative Disorders, Chronic |
| Extracranial Germ Cell Tumor, | Nasal Cavity and Paranasal Sinus Cancer |
| Childhood | Nasopharyngeal Cancer |
| Extragonadal Germ Cell Tumor | Nasopharyngeal Cancer, Childhood |
| Extrahepatic Bile Duct Cancer | Neuroblastoma |
| Eye Cancer, Intraocular Melanoma | Non-Hodgkin’s Lymphoma, Adult |
| Eye Cancer, Retinoblastoma | Non-Hodgkin’s Lymphoma, Childhood |
| Gallbladder Cancer | Non-Hodgkin’s Lymphoma During Pregnancy |
| Gastric (Stomach) Cancer | Non-Small Cell Lung Cancer |
| Gastric (Stomach) Cancer, Childhood | Oral Cancer, Childhood |
| Gastrointestinal Carcinoid Tumor | Oral Cavity Cancer, Lip and |
| Germ Cell Tumor, Extracranial, | Oropharyngeal Cancer |
| Childhood | Osteosarcoma/Malignant Fibrous |
| Germ Cell Tumor, Extragonadal | Histiocytoma of Bone |
| Germ Cell Tumor, Ovarian | Ovarian Cancer, Childhood |
| Gestational Trophoblastic Tumor | Ovarian Epithelial Cancer |
| Glioma, Adult | Ovarian Germ Cell Tumor |
| Glioma, Childhood Brain Stem | Ovarian Low Malignant Potential Tumor |
| Glioma, Childhood Cerebral | Pancreatic Cancer |
| Astrocytoma | Pancreatic Cancer, Childhood |
| Glioma, Childhood Visual Pathway and | Pancreatic Cancer, Islet Cell |
| Hypothalamic | Paranasal Sinus and Nasal Cavity Cancer |
| Skin Cancer (Melanoma) | Parathyroid Cancer |
| Skin Carcinoma, Merkel Cell | Penile Cancer |
| Small Cell Lung Cancer | Pheochromocytoma |
| Small Intestine Cancer | Pineoblastoma and Supratentorial Primitive |
| Soft Tissue Sarcoma, Adult | Neuroectodermal Tumors, Childhood |
| Soft Tissue Sarcoma, Childhood | Pituitary Tumor |
| Squamous Cell Carcinoma, see Skin | Plasma Cell Neoplasm/Multiple Myeloma |
| Cancer (non-Melanoma) | Pleuropulmonary Blastoma |
| Squamous Neck Cancer with Occult | Pregnancy and Breast Cancer |
| Primary, Metastatic | Pregnancy and Hodgkin’s Lymphoma |
| Stomach (Gastric) Cancer | Pregnancy and Non-Hodgkin’s Lymphoma |
| Stomach (Gastric) Cancer, Childhood | Primary Central Nervous System Lymphoma |
| Supratentorial Primitive | Prostate Cancer |
| Neuroectodermal Tumors, Childhood | Rectal Cancer |
| T-Cell Lymphoma, Cutaneous, see | Renal Cell (Kidney) Cancer |
| Mycosis Fungoides and Sézary | Renal Cell (Kidney) Cancer, Childhood |
| Syndrome | Renal Pelvis and Ureter, Transitional Cell |
| Testicular Cancer | Cancer |
| Thymoma, Childhood | Retinoblastoma |
| Thymoma and Thymic Carcinoma | Rhabdomyosarcoma, Childhood |
| Thyroid Cancer | Salivary Gland Cancer |
| Thyroid Cancer, Childhood | Salivary Gland Cancer, Childhood |
| Transitional Cell Cancer of the Renal | Sarcoma, Ewing’s Family of Tumors |
| Pelvis and Ureter | Sarcoma, Kaposi’s |
| Trophoblastic Tumor, Gestational | Sarcoma, Soft Tissue, Adult |
| Unknown Primary Site, Carcinoma of, | Sarcoma, Soft Tissue, Childhood |
| Adult | Sarcoma, Uterine |
| Unknown Primary Site, Cancer of, | Sezary Syndrome |
| Childhood | Skin Cancer (non-Melanoma) |
| Unusual Cancers of Childhood | Skin Cancer, Childhood |
| Ureter and Renal Pelvis, Transitional | Stomach Cancer |
| Cell Cancer | |
| Urethral Cancer | |
| Uterine Cancer, Endometrial | |
| Uterine Sarcoma | |
| Vaginal Cancer | |
| Visual Pathway and Hypothalamic | |
| Glioma, Childhood | |
| Vulvar Cancer | |
| Waldenström’s Macroglobulinemia | |
| Wilms’ Tumor | |

In another embodiment, multiple peptides of this invention together with at least one checkpoint inhibitor are used to stimulate an immune response in methods of the present invention.

As provided herein, heteroclitic peptides that elicit antigen-specific CD8⁺ T cell responses can be created using methods of the present invention. WT1 peptides that elicit CD4⁺ T cell responses to multiple HLA class II molecules can be identified. CD4⁺ T cells recognize peptides bound to the HLA class II molecule on APC. In another embodiment, antigen-specific CD4⁺ T cell responses assist in induction and maintenance of CD8⁺ cytotoxic T cell (CTL) responses.

In another embodiment, peptides of the present invention administered together with at least one checkpoint inhibitor exhibit an enhanced ability to elicit CTL responses, due to their ability to bind both HLA class I and HLA class II molecules. In another embodiment, peptides of the present invention administered together with at least one checkpoint inhibitor exhibit an enhanced ability to elicit CTL responses, due to the ability of the checkpoint inhibitor to increase the survival and proliferation of WT1 specific CTLs. In another embodiment, vaccines of the present invention administered together with at least one checkpoint inhibitor have the advantage of activating or eliciting both CD4⁺ and CD8⁺ T cells that recognize WT1 antigens. In another embodiment, activation or eliciting both CD4⁺ and CD8⁺ T cells provides a synergistic anti-WT1 immune response, relative to activation of either population alone. In another embodiment, the enhanced immunogenicity of peptides of the present invention is exhibited in individuals of multiple HLA class II subtypes, due to the ability of peptides of the present invention to bind multiple HLA class II subtypes. Each possibility represents a separate embodiment of the present invention.

In another embodiment, activated CD4⁺ cells enhance immunity by licensing dendritic cells, thereby sustaining the activation and survival of the cytotoxic T cells. In another embodiment, activated CD4⁺ T cells induce tumor cell death by direct contact with the tumor cell or by activation of the apoptosis pathway. Mesothelioma tumor cells, for example, are able to process and present antigens in the context of HLA class I and class II molecules.

The methods disclosed herein will be understood by those in the art to enable design of other WT1-derived peptides that are capable of binding both HLA class I and HLA class II molecules. The methods further enable design of immunogenic compositions and vaccines combining WT1-derived peptides of the present invention. Each possibility represents a separate embodiment of the present invention.

In another embodiment, methods, peptides, vaccines, and/or immunogenic compositions administered together with at least one checkpoint inhibitor of the present invention have the advantage of activating or eliciting WT1-specific CD4⁺ T cells containing multiple different HLA class II alleles. In another embodiment, the vaccines have the advantage of activating or eliciting WT1-specific CD4⁺ T cells in a substantial proportion of the population. In another embodiment, the peptides activate WT1-specific CD4⁺ T cells in 10% of the population. In another embodiment, the peptides activate WT1-specific CD4⁺ T cells in 15% of the population. In another embodiment, the peptides activate WT1-specific CD4⁺ T cells in 20% of the population. In another embodiment, the peptides activate WT1-specific CD4⁺ T cells in 25% of the population. In another embodiment, the peptides activate WT1-specific CD4⁺ T cells in 30% of the population. In another embodiment, the peptides activate WT1-specific CD4⁺ T cells in 35% of the population. In another embodiment, the peptides activate WT1-specific CD4⁺ T cells in 40% of the population. In another embodiment, the peptides activate WT1-specific CD4⁺ T cells in 45% of the population. In another embodiment, the peptides activate WT1-specific CD4⁺ T cells in 50% of the population. In another embodiment, the peptides activate WT1-specific CD4⁺ T cells in 55% of the population. In another embodiment, the peptides activate WT1-specific CD4⁺ T cells in 60% of the population. In another embodiment, the peptides activate WT1-specific CD4⁺ T cells in 70% of the population. In another embodiment, the peptides activate WT1-specific CD4⁺ T cells in 75% of the population. In another embodiment, the peptides activate WT1-specific CD4⁺ T cells in 80% of the population. In another embodiment, the peptides activate WT1-specific CD4⁺ T cells in 85% of the population. In another embodiment, the peptides activate WT1-specific CD4⁺ T cells in 90% of the population. In another embodiment, the peptides activate WT1-specific CD4⁺ T cells in 95% of the population. In another embodiment, the peptides activate WT1-specific CD4⁺ T cells in greater than 95% of the population. In another embodiment, the vaccines activate or elicit WT1-specific CD4⁺ T cells in a substantial proportion of a particular population (e.g. American Caucasians). Each possibility represents a separate embodiment of the present invention.

In another embodiment, methods of the present invention provide for an improvement in an immune response that has already been mounted by a subject. In another embodiment, methods of the present invention comprise administering the peptide, composition, or vaccine together with at least one checkpoint inhibitor one more time or two more times. In another embodiment, the peptides are varied in their composition, concentration, or a combination thereof. In another embodiment, the peptides administered together with at least one checkpoint inhibitor provide for the initiation of an immune response against an antigen of interest in a subject in which an immune response against the antigen of interest has not already been initiated. In another embodiment, the CTL that are induced proliferate in response to presentation of the peptide on the APC or cancer cell. In other embodiments, reference to modulation of the immune response involves, either or both the humoral and cell-mediated arms of the immune system, which is accompanied by the presence of Th2 and Th1 T helper cells, respectively, or in another embodiment, each arm individually.

In other embodiments, the methods affecting the growth of a tumor result in (1) the direct inhibition of tumor cell division, or (2) immune cell mediated tumor cell lysis, or both, which leads to a suppression in the net expansion of tumor cells. Each possibility represents a separate embodiment of the present invention. The use of the peptide or vaccine administered together with at least one checkpoint inhibitor increases the direct inhibition of tumor cell division, the immune cell mediated cell lysis, or both, greater than without the use of the checkpoint inhibitor.

Inhibition of tumor growth by either of these two mechanisms can be readily determined by one of ordinary skill in the art based upon a number of well-known methods. In another embodiment, tumor inhibition is determined by measuring the actual tumor size over a period of time. In another embodiment, tumor inhibition can be determined by estimating the size of a tumor (over a period of time) utilizing methods well known to those of skill in the art. More specifically, a variety of radiologic imaging methods (e.g., single photon and positron emission computerized tomography; see generally, “Nuclear Medicine in Clinical Oncology,” Winkler, C. (ed.) Springer-Verilog, New York, 1986), can be utilized to estimate tumor size. Such methods can also utilize a variety of imaging agents, including for example, conventional imaging agents (e.g., Gallium-67 citrate), as well as specialized reagents for metabolite imaging, receptor imaging, or immunologic imaging (e.g., radiolabeled monoclonal antibody specific tumor markers). In addition, non-radioactive methods such as ultrasound (see, “Ultrasonic Differential Diagnosis of Tumors”, Kossoff and Fukuda, (eds.), Igaku-Shoin, New York, 1984), can also be utilized to estimate the size of a tumor.

In addition to the in vivo methods for determining tumor inhibition discussed above, a variety of in vitro methods can be utilized in order to determine in vivo tumor inhibition. Representative examples include lymphocyte mediated anti-tumor cytolytic activity determined for example, by a ⁵¹Cr release assay, tumor dependent lymphocyte proliferation (Ioannides, et al., J. Immunol. 146(5):1700-1707, 1991), in vitro generation of tumor specific antibodies (Herlyn, et al., J. Immunol. Meth. 73:157-167, 1984), cell (e.g., CTL, helper T-cell) or humoral (e.g., antibody) mediated inhibition of cell growth in vitro (Gazit, et al., Cancer Immunol Immunother 35:135-144, 1992), and, for any of these assays, determination of cell precursor frequency (Vose, Int. J. Cancer 30:135-142 (1982), and others.

In another embodiment, methods of suppressing tumor growth indicate a growth state that is curtailed compared to growth without contact with, or exposure to a peptide administered together with at least one checkpoint inhibitor of this invention. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth refers, in other embodiments, to slowing, delaying, or stopping tumor growth, or to tumor shrinkage. Each possibility represents a separate embodiment of the present invention.

In another embodiment of methods and compositions of the present invention, WT1 expression is measured before administration of the treatment, after administration of the treatment, or both before and after administration of the treatment. In another embodiment, WT1 transcript expression is measured. In another embodiment, WT1 protein levels in the tumor or cancer cells are measured. In another embodiment, WT1 protein or peptides shed from cancer cells or tumor cells into circulation or other bodily fluids such as but not limited to urine are measured. Each possibility represents a separate embodiment of the present invention.

In another embodiment of methods and compositions of the invention, expression of the checkpoint protein(s) targeted by the one or more checkpoint inhibitors administered to the subject is measured (at the transcript level or protein level) in the tumor or cancer cells, or in whole blood, serum, or plasma, before administration of the treatment (baseline), after administration of the treat, or both before and after administration of the treatment. In one embodiment of methods and compositions of the invention, the one or more checkpoint proteins is selected from among: CTLA-4, PD-L1, PD-L2, PD1, B7-H3, B7-H4, BTLA, HVEM, TIM3, GALS, LAG3, VISTA, KIR, 2B4, CD160, CGEN-15049, CHK 1 kinase, CHK2 kinase, A2aR, and B-7 family ligands. In one embodiment of methods and compositions of the invention, expression of PD1, PD2, CTLA4, or a combination of two or more of the foregoing are measured before administration of the treatment, after administration of the treatment, or both before and after administration of the treatment. In one embodiment, checkpoint protein expression is measured at a primary tumor site. In another embodiment, the cancer is metastatic and the checkpoint protein expression is measured at a metastatic site, or the primary tumor site, or both.

In another embodiment of methods and compositions of the invention, one or more of the following markers are measured before administration of the treatment (baseline), after administration of the treatment, or both before and after administration of the treatment: monocytic myeloid-derived suppressor cells (m-MDSCs), C-reactive protein (CRP), absolute lymphocytes, absolute lymphocytes, and lactate dehydrogenase (LDH). In another embodiment, use of one or more markers for predicting or identifying responsiveness to checkpoint modulation is embraced herein.

Methods of determining the presence and magnitude of an immune response are well known in the art. In another embodiment, lymphocyte proliferation assays, wherein T cell uptake of a radioactive substance, e.g. ³H-thymidine is measured as a function of cell proliferation. In other embodiments, detection of T cell proliferation is accomplished by measuring increases in interleukin-2 (IL-2) production, Ca²⁺ flux, or dye uptake, such as 3-(4,5-dimethylthiazol-2-yl)-2,5-diphenyl-tetrazolium. Each possibility represents a separate embodiment of the present invention.

In another embodiment, CTL stimulation is determined by means known to those skilled in the art, including detection of cell proliferation, cytokine production and others. Analysis of the types and quantities of cytokines secreted by T cells upon contacting ligand-pulsed targets can be a measure of functional activity. Cytokines can be measured by ELISA, ELISPOT assays or fluorescence-activated cell sorting (FACS) to determine the rate and total amount of cytokine production. (Fujihashi K. et al. (1993) J. Immunol. Meth. 160:181; Tanguay S. and Killion J. J. (1994) Lymphokine Cytokine Res. 13:259).

In another embodiment, CTL activity is determined by ⁵¹Cr-release lysis assay. Lysis of peptide-pulsed ⁵¹Cr-labeled targets by antigen-specific T cells can be compared for target cells pulsed with control peptide. In another embodiment, T cells are stimulated with a peptide of this invention, and lysis of target cells expressing the native peptide in the context of MHC can be determined. The kinetics of lysis as well as overall target lysis at a fixed timepoint (e.g., 4 hours) are used, in another embodiment, to evaluate ligand performance. (Ware C. F. et al. (1983) J Immunol 131: 1312).

Methods of determining affinity of a peptide for an HLA molecule are well known in the art. In another embodiment, affinity is determined by TAP stabilization assays.

In another embodiment, affinity is determined by competition radioimmunoassay. In another embodiment, the following protocol is utilized: Target cells are washed two times in PBS with 1% bovine serum albumin (BSA; Fisher Chemicals, Fairlawn, N.J.). Cells are resuspended at 10⁷/ml on ice, and the native cell surface bound peptides are stripped for 2 minutes at 0° C. using citrate-phosphate buffer in the presence of 3 mg/ml beta₂ microglobulin. The pellet is resuspended at 5×10⁶ cells/ml in PBS/1% BSA in the presence of 3 mg/ml beta₂ microglobulin and 30 mg/ml deoxyribonuclease, and 200 ml aliquots are incubated in the presence or absence of HLA-specific peptides for 10 min at 20° C., then with ¹²⁵I-labeled peptide for 30 min at 20° C. Total bound ¹²⁵I is determined after two washes with PBS/2% BSA and one wash with PBS. Relative affinities are determined by comparison of escalating concentrations of the test peptide versus a known binding peptide.

In another embodiment, a specificity analysis of the binding of peptide to HLA on surface of live cells (e.g. SKLY-16 cells) is conducted to confirm that the binding is to the appropriate HLA molecule and to characterize its restriction. This includes, in another embodiment, competition with excess unlabeled peptides known to bind to the same or disparate HLA molecules and use of target cells which express the same or disparate HLA types. This assay is performed, in another embodiment, on live fresh or 0.25% paraformaldehyde-fixed human PBMC, leukemia cell lines and EBV-transformed T-cell lines of specific HLA types. The relative avidity of the peptides found to bind MHC molecules on the specific cells are assayed by competition assays as described above against ¹²⁵I-labeled peptides of known high affinity for the relevant HLA molecule, e.g., tyrosinase or HBV peptide sequence.

In another embodiment, any WT1 peptide used in the methods and compositions of the present invention comprises one or more non-classical amino acids such as: 1,2,3,4-tetrahydroisoquinoline-3-carboxylate (Kazmierski et al. (1991) J. Am Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski and Hruby (1991) Tetrahedron Lett. 32(41): 5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1984) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al. (1993) Int. J. Pep. Protein Res. 42(1):68-77) and ((1992) Acta. Cryst., Crystal Struc. Comm. 48(IV):1239-124). Such non-classical amino acids are embodied in the modified peptides of the invention.

In another embodiment, any peptide used in the methods and compositions of the present invention comprises one or more AA analogs or is a peptidomimetic, which, in other embodiments, induces or favors specific secondary structures. Such peptides comprise, in other embodiments, the following: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Left. 29:5057-5060); alpha-helix inducing analogs (Kemp et al. (1988) Tetrahedron Left. 29:4935-4938); gamma-turn inducing analogs (Kemp et al. (1989) J. Org. Chem. 54:109:115); analogs provided by the following references: Nagai and Sato (1985) Tetrahedron Left. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Jones et al. (1988) Tetrahedron Left. 29(31):3853-3856); tretrazol (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garvey et al. (1990) J. Org. Chem. 55(3):936-940. Conformationally restricted mimetics of beta turns and beta bulges, and peptides containing them, are described in U.S. Pat. No. 5,440,013, issued Aug. 8, 1995 to Kahn.

In other embodiments, any peptide used in a method of the invention is conjugated to one of various other molecules, as described hereinbelow, which can be via covalent or non-covalent linkage (complexed), the nature of which varies, in another embodiment, depending on the particular purpose. In another embodiment, the peptide is covalently or non-covalently complexed to a macromolecular carrier, (e.g. an immunogenic carrier), including, but not limited to, natural and synthetic polymers, proteins, polysaccharides, polypeptides (amino acids), polyvinyl alcohol, polyvinyl pyrrolidone, and lipids. In another embodiment, a peptide of this invention is linked to a substrate. In another embodiment, the peptide is conjugated to a fatty acid, for introduction into a liposome (U.S. Pat. No. 5,837,249). In another embodiment, a peptide of the invention is complexed covalently or non-covalently with a solid support, a variety of which are known in the art. In another embodiment, linkage of the peptide to the carrier, substrate, fatty acid, or solid support serves to increase an elicited an immune response.

In other embodiments, the carrier is thyroglobulin, an albumin (e.g. human serum albumin), tetanus toxoid, polyamino acids such as poly (lysine: glutamic acid), an influenza protein, hepatitis B virus core protein, keyhole limpet hemocyanin, an albumin, or another carrier protein or carrier peptide; hepatitis B virus recombinant vaccine, or an APC. Each possibility represents a separate embodiment of the present invention.

In another embodiment, the term “amino acid” refers to a natural or, in another embodiment, an unnatural or synthetic AA, and can include, in other embodiments, glycine, D- or L optical isomers, AA analogs, peptidomimetics, or combinations thereof.

In another embodiment, the terms “cancer,” “neoplasm,” “neoplastic” or “tumor,” are used interchangeably and refer to cells that have undergone a malignant transformation that makes them pathological to the host organism. The cancer may be of any stage within the numbered staging system (e.g., stage 0, stage 1, stage 2, stage 3, or stage 4), and any stage in the TNM staging system. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. In another embodiment, a tumor is detectable on the basis of tumor mass; e.g., by such procedures as CAT scan, magnetic resonance imaging (MRI), X-ray, ultrasound or palpation, and in another embodiment, is identified by biochemical or immunologic findings, the latter which is used to identify cancerous cells, as well, in other embodiments. A tumor may be a solid tumor or non-solid tumor.

Methods for synthesizing peptides are well known in the art. In another embodiment, the peptides of this invention are synthesized using an appropriate solid-state synthetic procedure (see for example, Steward and Young, Solid Phase Peptide Synthesis, Freemantle, San Francisco, Calif. (1968); Merrifield (1967) Recent Progress in Hormone Res 23: 451). The activity of these peptides is tested, in other embodiments, using assays as described herein.

In another embodiment, the peptides of this invention are purified by standard methods including chromatography (e.g., ion exchange, affinity, and sizing column chromatography), centrifugation, differential solubility, or by any other standard technique for protein purification. In another embodiment, immuno-affinity chromatography is used, whereby an epitope is isolated by binding it to an affinity column comprising antibodies that were raised against that peptide, or a related peptide of the invention, and were affixed to a stationary support.

In another embodiment, affinity tags such as hexa-His (Invitrogen), Maltose binding domain (New England Biolabs), influenza coat sequence (Kolodziej et al. (1991) Meth. Enzymol. 194:508-509), glutathione-S-transferase, or others, are attached to the peptides of this invention to allow easy purification by passage over an appropriate affinity column. Isolated peptides can also be physically characterized, in other embodiments, using such techniques as proteolysis, nuclear magnetic resonance, and x-ray crystallography.

In another embodiment, the peptides of this invention are produced by in vitro translation, through known techniques, as will be evident to one skilled in the art. In another embodiment, the peptides are differentially modified during or after translation, e.g., by phosphorylation, glycosylation, cross-linking, acylation, proteolytic cleavage, linkage to an antibody molecule, membrane molecule or other ligand, (Ferguson et al. (1988) Ann. Rev. Biochem. 57:285-320).

In another embodiment, the peptides of this invention further comprise a detectable label, which in another embodiment, is fluorescent, or in another embodiment, luminescent, or in another embodiment, radioactive, or in another embodiment, electron dense. In other embodiments, the dectectable label comprises, for example, green fluorescent protein (GFP), DS-Red (red fluorescent protein), secreted alkaline phosphate (SEAP), beta-galactosidase, luciferase, ³²P, ¹²⁵I, ³H and ¹⁴C, fluorescein and its derivatives, rhodamine and its derivatives, dansyl and umbelliferone, luciferin or any number of other such labels known to one skilled in the art. The particular label used will depend upon the type of immunoassay used.

In another embodiment, a peptide of this invention is linked to a substrate, which, in another embodiment, serves as a carrier. In another embodiment, linkage of the peptide to a substrate serves to increase an elicited an immune response.

In another embodiment, peptides of this invention are linked to other molecules, as described herein, using conventional cross-linking agents such as carbodimides. Examples of carbodimides are 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-44-dimethylpentyl) carbodiimide.

In other embodiments, the cross-linking agents comprise cyanogen bromide, glutaraldehyde and succinic anhydride. In general, any of a number of homo-bifunctional agents including a homo-bifunctional aldehyde, a homo-bifunctional epoxide, a homo-bifunctional imido-ester, a homo-bifunctional N-hydroxysuccinimide ester, a homo-bifunctional maleimide, a homo-bifunctional alkyl halide, a homo-bifunctional pyridyl disulfide, a homo-bifunctional aryl halide, a homo-bifunctional hydrazide, a homo-bifunctional diazonium derivative and a homo-bifunctional photoreactive compound can be used. Also envisioned, in other embodiments, are hetero-bifunctional compounds, for example, compounds having an amine-reactive and a sulfhydryl-reactive group, compounds with an amine-reactive and a photoreactive group and compounds with a carbonyl-reactive and a sulfhydryl-reactive group.

In other embodiments, the homo-bifunctional cross-linking agents include the bifunctional N-hydroxysuccinimide esters dithiobis(succinimidylpropionate), disuccinimidyl suberate, and disuccinimidyl tartarate; the bifunctional imido-esters dimethyl adipimidate, dimethyl pimelimidate, and dimethyl suberimidate; the bifunctional sulfhydryl-reactive crosslinkers 1,4-di-[3′-(2′-pyridyldithio)propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri(2-chloroethyl)amine, respectively.

In other embodiments, hetero-bifunctional cross-linking agents used to link the peptides to other molecules, as described herein, include, but are not limited to, SMCC (succinimidyl-4-(N-maleimidomethyl)cyclohexane-1-carboxylate), MBS (m-maleimidobenzoyl-N-hydroxysuccinimide ester), SIAB (N-succinimidyl(4-iodoacteyl)aminobenzoate), SMPB (succinimidyl-4-(p-maleimidophenyl)butyrate), GMBS (N-(.gamma.-maleimidobutyryloxy)succinimide ester), MPBH (4-(4-N-maleimidopohenyl) butyric acid hydrazide), M2C2H (4-(N-maleimidomethyl) cyclohexane-1-carboxyl-hydrazide), SMPT (succinimidyloxycarbonyl-a-methyl-a-(2-pyridyldithio)toluene), and SPDP (N-succinimidyl 3-(2-pyridyldithio)propionate).

In another embodiment, the peptides of the invention are formulated as non-covalent attachment of monomers through ionic, adsorptive, or biospecific interactions. Complexes of peptides with highly positively or negatively charged molecules can be accomplished, in another embodiment, through salt bridge formation under low ionic strength environments, such as in deionized water. Large complexes can be created, in another embodiment, using charged polymers such as poly-(L-glutamic acid) or poly-(L-lysine), which contain numerous negative and positive charges, respectively. In another embodiment, peptides are adsorbed to surfaces such as microparticle latex beads or to other hydrophobic polymers, forming non-covalently associated peptide-superantigen complexes effectively mimicking cross-linked or chemically polymerized protein, in other embodiments. In another embodiment, peptides are non-covalently linked through the use of biospecific interactions between other molecules. For instance, utilization of the strong affinity of biotin for proteins such as avidin or streptavidin or their derivatives could be used to form peptide complexes. The peptides, according to this aspect, and in another embodiment, can be modified to possess biotin groups using common biotinylation reagents such as the N-hydroxysuccinimidyl ester of D-biotin (NHS-biotin), which reacts with available amine groups.

In another embodiment, a peptide of the present invention is linked to a carrier. In another embodiment, the carrier is KLH. In other embodiments, the carrier is any other carrier known in the art, including, for example, thyroglobulin, albumins such as human serum albumin, tetanus toxoid, polyamino acids such as poly (lysine:glutamic acid), influenza, hepatitis B virus core protein, hepatitis B virus recombinant vaccine and the like. Each possibility represents a separate embodiment of the present invention.

In another embodiment, the peptides of this invention are conjugated to a lipid, such as P3 CSS. In another embodiment, the peptides of this invention are conjugated to a bead.

In any of the foregoing embodiments, the peptide, cross-linked peptide, bound peptide or any other form of the peptide is used in a method of the invention together with at least one checkpoint inhibitor.

In another embodiment, in addition to the use of at least one checkpoint inhibitor, the methods and compositions of this invention further comprise immunomodulating compounds. In other embodiments, the immunomodulating compound is a cytokine, chemokine, or complement component that enhances expression of immune system accessory or adhesion molecules, their receptors, or combinations thereof. In some embodiments, the immunomodulating compound include interleukins, for example interleukins 1 to 15, interferons alpha, beta or gamma, tumor necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIP-1a and MIP-1b, complement components, or combinations thereof. In other embodiments, the immunomodulating compound stimulate expression, or enhanced expression of OX40, OX40L (gp34), lymphotactin, CD40, CD40L, B7.1, B7.2, TRAP, ICAM-1, 2 or 3, cytokine receptors, or combination thereof.

In another embodiment, the immunomodulatory compound induces or enhances expression of co-stimulatory molecules that participate in the immune response, which include, in some embodiments.

In one embodiment, patients administered the WT1 vaccine and the checkpoint inhibitor in accordance with the invention also are administered GM-CSF prior to or on the day of first vaccination, or the combination thereof. In one embodiment, a patient is administered 70 mcg of GM-CSF subcutaneously two days before and on the day of first vaccine administration.

In another embodiment, the composition comprises a solvent, including water, dispersion media, cell culture media, isotonic agents and the like. In another embodiment, the solvent is an aqueous isotonic buffered solution with a pH of around 7.0. In another embodiment, the composition comprises a diluent such as water, phosphate buffered saline, or saline. In another embodiment, the composition comprises a solvent, which is non-aqueous, such as propyl ethylene glycol, polyethylene glycol and vegetable oils.

In another embodiment, the composition is formulated for administration by any of the many techniques known to those of skill in the art. For example, this invention provides for administration of the pharmaceutical composition parenterally, intravenously, subcutaneously, intradermally, intramucosally, topically, orally, or by inhalation.

In another embodiment, in the uses of the vaccine comprising one or more WT1 delivery agents to deliver the combination of at least seven WT1 peptides, or CTLs induced by the at least seven WT1 peptides, of this invention, the vaccine may further comprise a cell population, which, in another embodiment, comprises lymphocytes, monocytes, macrophages, dendritic cells, endothelial cells, stem cells or combinations thereof, which, in another embodiment are autologous, syngeneic or allogeneic, with respect to each other. In another embodiment, the cell population comprises a peptide of the present invention. In another embodiment, the cell population takes up the peptide. In one embodiment, the cell is an antigen presenting cell (APC). In a further embodiment, the APC is a professional APC. Each possibility represents a separate embodiment of the present invention.

In another embodiment, the cell populations of this invention are obtained from in vivo sources, such as, for example, peripheral blood, leukopheresis blood product, apheresis blood product, peripheral lymph nodes, gut associated lymphoid tissue, spleen, thymus, cord blood, mesenteric lymph nodes, liver, sites of immunologic lesions, e.g. synovial fluid, pancreas, cerebrospinal fluid, tumor samples, granulomatous tissue, or any other source where such cells can be obtained. In another embodiment, the cell populations are obtained from human sources, which are, in other embodiments, from human fetal, neonatal, child, or adult sources. In another embodiment, the cell populations of this invention are obtained from animal sources, such as, for example, porcine or simian, or any other animal of interest. In another embodiment, the cell populations of this invention are obtained from subjects that are normal, or in another embodiment, diseased, or in another embodiment, susceptible to a disease of interest.

In another embodiment, the cell populations of this invention are separated via affinity-based separation methods. Techniques for affinity separation include, in other embodiments, magnetic separation, using antibody-coated magnetic beads, affinity chromatography, cytotoxic agents joined to a monoclonal antibody or use in conjunction with a monoclonal antibody, for example, complement and cytotoxins, and “panning” with an antibody attached to a solid matrix, such as a plate, or any other convenient technique. In other embodiment, separation techniques include the use of fluorescence activated cell sorters, which can have varying degrees of sophistication, such as multiple color channels, low angle and obtuse light scattering detecting channels, impedance channels, etc. In other embodiments, any technique that enables separation of the cell populations of this invention can be employed, and is to be considered as part of this invention.

In another embodiment, the dendritic cells are from the diverse population of morphologically similar cell types found in a variety of lymphoid and non-lymphoid tissues, qualified as such (Steinman (1991) Ann. Rev. Immunol. 9:271-296). In another embodiment, the dendritic cells used in this invention are isolated from bone marrow, or in another embodiment, derived from bone marrow progenitor cells, or, in another embodiment, from isolated from/derived from peripheral blood, or in another embodiment, derived from, or are a cell line.

In another embodiment, the cell populations described herein are isolated from the white blood cell fraction of a mammal, such as a murine, simian or a human (See, e.g., WO 96/23060). The white blood cell fraction can be, in another embodiment, isolated from the peripheral blood of the mammal.

Methods of isolating dendritic cells are well known in the art. In another embodiment, the DC are isolated via a method which includes the following steps: (a) providing a white blood cell fraction obtained from a mammalian source by methods known in the art such as leukophoresis; (b) separating the white blood cell fraction of step (a) into four or more subfractions by countercurrent centrifugal elutriation; (c) stimulating conversion of monocytes in one or more fractions from step (b) to dendritic cells by contacting the cells with calcium ionophore, GM-CSF and IL-13 or GM-CSF and IL-4, (d) identifying the dendritic cell-enriched fraction from step (c); and (e) collecting the enriched fraction of step (d), preferably at about 4° C.

In another embodiment, the dendritic cell-enriched fraction is identified by fluorescence-activated cell sorting, which identifies, in another embodiment, at least one of the following markers: HLA-DR, HLA-DQ, or B7.2, and the simultaneous absence of the following markers: CD3, CD14, CD16, 56, 57, and CD 19, 20.

In another embodiment, the cell population comprises lymphocytes, which are, in another embodiment, T cells, or in another embodiment, B cells. The T cells are, in other embodiments, characterized as NK cells, helper T cells, cytotoxic T lymphocytes (CTL), TILs, naïve T cells, or combinations thereof. It is to be understood that T cells which are primary, or cell lines, clones, etc. are to be considered as part of this invention. In another embodiment, the T cells are CTL, or CTL lines, CTL clones, or CTLs isolated from tumor, inflammatory, or other infiltrates.

In another embodiment, hematopoietic stem or early progenitor cells comprise the cell populations used in this invention. In another embodiment, such populations are isolated or derived, by leukapheresis. In another embodiment, the leukapheresis follows cytokine administration, from bone marrow, peripheral blood (PB) or neonatal umbilical cord blood. In another embodiment the stem or progenitor cells are characterized by their surface expression of the surface antigen marker known as CD34⁺, and exclusion of expression of the surface lineage antigen markers, Lin−.

In another embodiment, the subject is administered a peptide, composition or vaccine of this invention, in conjunction with bone marrow cells. In another embodiment, the administration together with bone marrow cells embodiment follows previous irradiation of the subject, as part of the course of therapy, in order to suppress, inhibit or treat cancer in the subject.

In another embodiment, the phrase “contacting a cell” or “contacting a population” refers to a method of exposure, which can be, in other embodiments, direct or indirect. In another embodiment, such contact comprises direct injection of the cell through any means well known in the art, such as microinjection. It is also envisaged, in another embodiment, that supply to the cell is indirect, such as via provision in a culture medium that surrounds the cell, or administration to a subject, via any route well known in the art, and as described herein.

In another embodiment, CTL generation of methods of the present invention is accomplished in vivo, and is effected by introducing into a subject an antigen presenting cell contacted in vitro with a peptide of this invention (See for example Paglia et al. (1996) J. Exp. Med. 183:317-322), administered together with at least one checkpoint inhibitor.

In another embodiment, the peptides of methods and compositions of the present invention are delivered to antigen-presenting cells (APC).

In another embodiment, the peptides are delivered to APC in the form of cDNA encoding the peptides. In another embodiment, the term “antigen-presenting cells” refers to dendritic cells (DC), monocytes/macrophages, B lymphocytes or other cell type(s) expressing the necessary MHC/co-stimulatory molecules, which effectively allow for T cell recognition of the presented peptide. In another embodiment, the APC is a cancer cell. Each possibility represents a separate embodiment of the present invention. In each embodiment, the vaccine or APC or any form of peptide delivery to the patient or subject is administered together with at least one checkpoint inhibitor. As noted herein, the administration of the at least one checkpoint inhibitor does not need to be in the same vaccine, formulation, administration site or time of administration of the WT1 vaccine or its alternate forms. As embodied herein, the administration of the checkpoint inhibitor contemporaneously with the WT1 vaccine, in any of its various forms, enhances the formation of WT1-specific CTLs in the subject in need thereof.

In another embodiment, the CTL are contacted with two or more antigen-presenting cell populations, together with at least one checkpoint inhibitor. In another embodiment, the two or more antigen presenting cell populations present different peptides. Each possibility represents a separate embodiment of the present invention.

In another embodiment, techniques that lead to the expression of antigen in the cytosol of APC (e.g. DC) are used to deliver the peptides to the APC. Methods for expressing antigens on APC are well known in the art. In another embodiment, the techniques include (1) the introduction into the APC of naked DNA encoding a peptide of this invention, (2) infection of APC with recombinant vectors expressing a peptide of this invention, and (3) introduction of a peptide of this invention into the cytosol of an APC using liposomes. (See Boczkowski D. et al. (1996) J. Exp. Med. 184:465-472; Rouse et al. (1994) J. Virol. 68:5685-5689; and Nair et al. (1992) J. Exp. Med. 175:609-612).

In another embodiment, foster antigen presenting cells such as those derived from the human cell line 174xCEM.T2, referred to as T2, which contains a mutation in its antigen processing pathway that restricts the association of endogenous peptides with cell surface MHC class I molecules (Zweerink et al. (1993) J. Immunol. 150:1763-1771), are used, as exemplified herein.

In another embodiment, any of the methods described herein is used to elicit CTL, which are elicited in vitro. In another embodiment, the CTL are elicited ex-vivo. In another embodiment, the CTL are elicited in vitro. The resulting CTL, are, in another embodiment, administered to the subject, thereby treating the condition associated with the peptide, an expression product comprising the peptide, or a homologue thereof, administered together with at least one checkpoint inhibitor. Each possibility represents a separate embodiment of the present invention.

In another embodiment, the methods of the invention entail introduction of the genetic sequence that encodes the combination of at least seven WT1 peptides of this invention. The nucleic acid may be included within one or more vectors; thus, in another embodiment, the method comprises administering to the subject a vector comprising a nucleotide sequence, which encodes a peptide of the present invention (Tindle, R. W. et al. Virology (1994) 200:54). In another embodiment, the method comprises administering to the subject naked nucleic acid (DNA or RNA) which encodes a peptide, or in another embodiment, two or more peptides of this invention (Nabel, et al. PNAS-USA (1990) 90: 11307). In another embodiment, multi-epitope, analogue-based cancer vaccines are utilized (Fikes et al., Expert Opin Biol Ther., 2003, September; 3(6):985-993). Each possibility represents a separate embodiment of the present invention. In each of the foregoing embodiments, the nucleic acid can encode each WT1 peptide individually, or combinations of up to seven or more of the WT1 peptides. Nucleic acids encoding WT1 peptides represent one form of WT1 delivery agent. The combination of at least seven WT1 peptides may be delivered by one form of WT1 delivery agent, such as peptides, or nucleic acids, or immune cells, or any combination of two or three of the foregoing.

A nucleic acid may encode a single WT1 peptide of the seven WT1 peptides, or a nucleic acid may encode a plurality of the seven WT1 peptides (e.g., two, three, four, five, six, or all seven WT1 peptides). Likewise, a nucleic acid may encode one or more of additional WT1 peptides, if utilized. Thus, the compositions and methods of the invention may use a single nucleic acid or multiple nucleic acids to serve as WT1 delivery agents. The compositions and methods of the invention may use a single vector to deliver the at least seven WT1 peptides or a plurality of vectors.

Nucleic acids (DNA or RNA) can be administered to a subject via any means as is known in the art, including parenteral or intravenous administration, or in another embodiment, by means of a gene gun. In another embodiment, the nucleic acids are administered in a composition, which correspond, in other embodiments, to any embodiment listed herein. DNA or RNA can be administered to a subject as a naked nucleic acid or carried by a vector.

Vectors for use according to methods of this invention can comprise, in another embodiment, any vector that facilitates or allows for the expression of a peptide of this invention (e.g., one or more of the at least seven WT1 peptides) in a cell in vitro or in a subject in vivo. The term “vector” is used to refer to any molecule (e.g., nucleic acid, plasmid, virus, particle) usable to transfer coding sequence information (e.g., nucleic acid sequence encoding a WT1 peptide) to a cell or subject. Nucleic acid vaccines for several cancers have entered clinical trials (Wahren B et al., “DNA Vaccines: Recent Developments and the Future,” Vaccines, 2014, 2:785-796; Fioretti D. et al., “DNA Vaccines: Developing New Strategies Against Cancer, Journal of Biomedicine and Biotechnology, 2010, 2010(938):174378). Strategies for expanding functional WT1-specific T cells using a DNA vaccine are known (Chaise C et al., “DNA vaccination induces WT1-specific T-cell responses with potential clinical relevance,” Blood, 2008, 112(7):2956-2964). In one embodiment, the vector is a viral vector. In another embodiment, the vector is a non-viral vector. In one embodiment the non-viral vector is a nucleic acid vector such as plasmid DNA or mRNA vector (see, for example, Weide B. et al, “Plasmid DNA- and messenger RNA-based Anti-Cancer Vaccination,” Immunol Lett, 2008, 115(1):33-42); Kim H. et al., “Self-Assembled Messenger RNA Nanoparticles (mRNA-NPs) for Efficient Gene Expression,” Sci Rep, 2015, 5:12737); Ulmer J. B. et al. “RNA-based Vaccines”, Vaccine, 2012, 30:4414-4418). In another embodiment, “vectors” includes attenuated viruses, such as vaccinia or fowlpox, such as described in, e.g., U.S. Pat. No. 4,722,848, incorporated herein by reference. In another embodiment, the vector is BCG (Bacille Calmette Guerin), such as described in Stover et al. (Nature 351:456-460 (1991)). Other vectors useful for therapeutic administration or immunization of the peptides of the invention, e.g., Salmonella typhi vectors and the like, will be apparent to those skilled in the art from the description herein. Non-limiting examples of vectors that may be used to administer nucleic acid molecules to subjects in vivo and cells in vitro include adenovirus, adeno-associated virus, retrovirus, lentivirus, pox virus, herpes virus, virus-like particles (VLPs), plasmids, cationic lipids, liposomes, and nanoparticles.

A “coding sequence” is a nucleic acid sequence that is transcribed into mRNA and/or translated into a polypeptide. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences. Variants or analogs may be prepared by the deletion of a portion of the coding sequence, by insertion of a sequence, and/or by substitution of one or more nucleotides within the sequence. Techniques for modifying nucleic acid sequences, such as site-directed mutagenesis, are well known to those skilled in the art (See, e.g., Sambrook et al., Molecular Cloning: A Laboratory Manual, Second Edition, 1989; DNA Cloning, Vols. I and II, D. N. Glover ed., 1985). Optionally, the nucleic acid sequences of the present invention, and composition and methods of the invention that utilize such polynucleotides, can include non-coding sequences.

The term “operably-linked” is used herein to refer to an arrangement of flanking control sequences wherein the flanking sequences so described are configured or assembled so as to perform their usual function. Thus, a flanking control sequence operably-linked to a coding sequence may be capable of effecting the replication, transcription and/or translation of the coding sequence under conditions compatible with the control sequences. For example, a coding sequence is operably-linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence, and the promoter sequence can still be considered “operably-linked” to the coding sequence. Each nucleic acid sequence coding for a polypeptide (e.g., a WT1 peptide) will typically have its own operably-linked promoter sequence.

In another embodiment, the vector further encodes for an immunomodulatory compound, as described herein. In another embodiment, the subject is administered an additional vector encoding same, concurrent, prior to or following administration of the vector encoding a peptide of this invention to the subject.

In another embodiment, the WT1 delivery agents, CTLs, compositions, and vaccines of this invention are administered to a subject, or utilized in the methods of this invention, in combination with other anti-cancer compounds and chemotherapeutics, including monoclonal antibodies directed against alternate cancer antigens, or, in another embodiment, epitopes that consist of an AA sequence which corresponds to, or in part to, that from which the peptides of this invention are derived. This is in addition to the use of at least one checkpoint inhibitor in the practice of the various embodiments of the invention.

In another embodiment, the present invention provides a method of detecting a WT1-specific CD4⁺ T cell response in a subject, the method comprising administering to the subject a WT1 delivery agent, vaccine, or composition of the present invention. In another embodiment, a delayed-type hypersensitivity test used to detect the WT1-specific CD4⁺ T cell response. In another embodiment, a peptide of present invention is superior to its unmutated counterpart in inducing a CD4⁺ T cell response in a subject. Each possibility represents a separate embodiment of the present invention.

As used herein, the terms “patient”, “subject”, and “individual” are used interchangeably and are intended to include human and non-human animal species. For example, the subject may be a human or non-human mammal. In some embodiments, the subject is a non-human animal model or veterinary patient. The subject may be any age or gender.

An immunogenic composition of methods and compositions of the present invention comprises, in another embodiment, an APC associated with one or more WT1 delivery agents and/or CTLs of the present invention. In another embodiment, the immunogenic composition consists of an APC associated with one or more WT1 delivery agents and/or CTLs of the present invention. In another embodiment, the immunogenic composition comprises or consists of an APC associated with the combination of at least seven WT1 peptides.

A composition of methods and compositions of the present invention is, in another embodiment, an immunogenic composition. In another embodiment, the composition is a pharmaceutical composition. In another embodiment, the composition is any other type of composition known in the art. Each possibility represents a separate embodiment of the present invention. Each composition further comprises at least one checkpoint inhibitor.

Various embodiments of dosage ranges are contemplated by this invention. In another embodiment, the dosage is 20 μg per peptide per day. In another embodiment, the dosage is 10 μg/peptide/day. In another embodiment, the dosage is 30 μg/peptide/day. In another embodiment, the dosage is 40 μg/peptide/day. In another embodiment, the dosage is 60 μg/peptide/day. In another embodiment, the dosage is 80 μg/peptide/day. In another embodiment, the dosage is 100 μg/peptide/day. In another embodiment, the dosage is 150 μg/peptide/day. In another embodiment, the dosage is 200 μg/peptide/day. In another embodiment, the dosage is 300 μg/peptide/day. In another embodiment, the dosage is 400 μg/peptide/day. In another embodiment, the dosage is 600 μg/peptide/day. In another embodiment, the dosage is 800 μg/peptide/day. In another embodiment, the dosage is 1000 μg/peptide/day.

In another embodiment, the dosage is 10 μg/peptide/dose. In another embodiment, the dosage is 30 μg/peptide/dose. In another embodiment, the dosage is 40 μg/peptide/dose. In another embodiment, the dosage is 60 μg/peptide/dose. In another embodiment, the dosage is 80 μg/peptide/dose. In another embodiment, the dosage is 100 μg/peptide/dose. In another embodiment, the dosage is 150 μg/peptide/dose. In another embodiment, the dosage is 200 μg/peptide/dose. In another embodiment, the dosage is 300 μg/peptide/dose. In another embodiment, the dosage is 400 μg/peptide/dose. In another embodiment, the dosage is 600 μg/peptide/dose. In another embodiment, the dosage is 800 μg/peptide/dose. In another embodiment, the dosage is 1000 μg/peptide/dose.

In another embodiment, the dosage is 10-20 μg/peptide/dose. In another embodiment, the dosage is 20-30 μg/peptide/dose. In another embodiment, the dosage is 20-40 μg/peptide/dose. In another embodiment, the dosage is 30-60 μg/peptide/dose. In another embodiment, the dosage is 40-80 μg/peptide/dose. In another embodiment, the dosage is 50-100 μg/peptide/dose. In another embodiment, the dosage is 50-150 μg/peptide/dose. In another embodiment, the dosage is 100-200 μg/peptide/dose. In another embodiment, the dosage is 200-300 μg/peptide/dose. In another embodiment, the dosage is 300-400 μg/peptide/dose. In another embodiment, the dosage is 400-600 μg/peptide/dose. In another embodiment, the dosage is 500-800 μg/peptide/dose. In another embodiment, the dosage is 800-1000 μg/peptide/dose.

In another embodiment, the total amount of peptide per dose or per day is one of the above amounts. In another embodiment, the total peptide dose per dose is one of the above amounts.

Each of the above doses represents a separate embodiment of the present invention.

In another embodiment, the present invention provides a kit comprising a peptide, composition or vaccine of the present invention, together with at least one checkpoint inhibitor. In another embodiment, the kit further comprises a label or packaging insert. In another embodiment, the kit is used for detecting a WT1-specific CD4 response through the use of a delayed-type hypersensitivity test. In another embodiment, the kit is used for any other method enumerated herein. In another embodiment, the kit is used for any other method known in the art. Each possibility represents a separate embodiment of the present invention.

### Example

Evaluation of Efficacy of Heptavalent WT1 Immunotherapy Composition Administered Together with Nivolumab in Patients with Ovarian Cancer

Eligible patients diagnosed with ovarian cancer will start the vaccination schedule within 4 months of completion of chemotherapy. Patients will initially receive 6 vaccinations of WT1 peptides over 12 weeks, and 7 infusions of the immune checkpoint inhibitor nivolumab over 14 weeks. Toxicity assessments will be performed with each dose of vaccine, and 3 weeks after the completion of therapy at week 15. Patients will be observed by the study staff for up to 30 minutes following treatment. No dose escalation is planned. Routine toxicity assessments will continue throughout the trial.

Patients who do not have disease progression at the week 15 evaluation are permitted to receive 4 additional vaccines administered approximately every 8 weeks. This maintenance vaccine course would begin at week 19.

Immune responses will be evaluated from 40 ml heparinized blood samples at 6 separate time-points: baseline (at consent and before first dose in order to determine baseline variations), before vaccines 5 and 6 as well as 3 weeks after the last nivolumab infusion. If feasible, an additional blood draw will be obtained at the 3-month follow-up. Using ELISA, antibody levels generated against the 4 WT1 peptides in the vaccine will be measured. Antibodies are generally present by completion of the fourth vaccination. T-cell proliferative response assays will be performed on peripheral blood lymphocytes including: flow cytometry for phenotypic analysis with FACS including leukocyte subset analysis, T regulatory cell assay (including CD3, CD4, CD8, FOXP3, ICOS and PD1) and myeloid derived suppressor cells (MDSCs, CD14+HLA-DRlow cells) in peripheral blood and also in tumor (if optional biopsy obtained). WT1 T cell specific CD4 and CD8 proliferative response will be measured using polyfunctional intracellular cytokine staining (ICS) and flow cytometric based cytotoxicity assays using Meso Scale Discovery System with functionality measured by IFN-gamma production. Detailed procedures for blood sample processing, T cell monitoring, antibody ELISA and polyfunctional T cell assay, are described in [29].

Baseline values and T cell response results will be correlated with duration of clinical remission.

If a patient is removed from study prior to week 15, blood for post study immunologic studies will be obtained. A CT scan will be performed at baseline and week 15 (or sooner if deemed medically necessary) and every 3 months thereafter for up to 1 year until disease progression. MM abdomen and pelvis may be used in lieu of the CT abdomen and pelvis. The reference radiologist will use immune-related response criteria to determine disease progression [57]. CA125 will be obtained at baseline, weeks 6 and 15 and then every 3 months thereafter for up to 1 year until disease progression. CA125 will not be used to determine disease progression due to the confounding possibility of inflammation in vaccinated patients. Patients will remain on study until the time of progression, development of unacceptable toxicity, completion of the vaccine sequence or patient withdrawal.

WT1 Vaccine: The vaccine that will be used in this study contains seven separate WT1 peptides:
YMFPNAPYL (SEQ ID NO:124; WT1-A1): HLA class I peptide with a mutated amino acid R126Y to stimulate CD8+ responses.
SGQAYMFPNAPYLPSCLES (SEQ ID NO:125; WT1-122A1 long): HLA class II peptide containing an embedded WT1-A1 heteroclitic sequence within the longer peptide to stimulate both CD4+ and CD8+ responses according to data from preclinical and phase 1 studies.
RSDELVRHHNMHQRNMTKL (SEQ ID NO:1; WT1-427 long) and PGCNKRYFKLSHLQMHSRKHTG (SEQ ID NO:2; WT1-331 long): HLA class II peptides inducing CD4+ responses that could provide help for long lasting CD8+ T cell responses.
NLMNLGATL (SEQ ID NO: 21; NLM short),
WNLMNLGATLKGVAA (SEQ ID NO: 26; NLM long), and
WNYMNLGATLKGVAA (SEQ ID NO: 205; NYM long).

Drug Product: The seven peptides are provided in a sterile solution with phosphate buffered saline to produce the vaccine product (“WT1 Vax”). Each vial contains 280 mcg of each peptide in a total volume of 0.7 ml (0.4 mg/ml of each peptide, overfill of 40%). Vialing under GMP conditions and sterility testing was performed. The vaccine emulsion will be individually prepared prior to use. This will require mixture of the peptide solution with the immunologic adjuvant Montanide ISA 51 VG.

Intended Dose: The 200 mcg dose for each peptide is chosen because it is within the range of safe and active doses used by others. Peptide vaccines have generated immune and clinical responses within a wide range of doses (100-2000 mcg injected) without clear evidence of dose-response relationships. Higher doses have the theoretical possibility of stimulating lower affinity TCRs on T cells and making a reduced response [30, 33, 34]. Vial Size: Each single-dose vial contains 0.7 ml Route of Administration: Subcutaneous

Nivolumab: Intended Dose: 3 mg/kg; Vial Size: 10 mL; Route of Administration: Intravenous. Nivolumab will be dosed at 3 mg/kg and administered intravenously as a 60-minute IV infusion once every 2 weeks. At the end of the infusion, flush the line with a sufficient quantity of normal saline. If the subject's weight differs >10% from the previous weight used to calculate the required dose, a required dose, a corrected dose should be calculated. There will be no dose escalations or reductions of nivolumab allowed. There are no premedications recommended for the first nivolumab treatment.

Subjects may be dosed no less than 12 days between nivolumab doses and no more than 3 days after the scheduled dosing date. Dose given after the 3 days window is considered a dose delay. Treatment may be delayed for up to a maximum of 6 weeks from the previous dose.

Tumor assessments by CT or MM should continue as per protocol even if dosing is delayed.

### Treatment/Intervention Plan

Patients will be treated as outpatients.
WT1 vaccines will be administered on weeks 0, 2, 4, 6, 8 and 10.
All injections will be administered subcutaneously with sites rotating between extremities.
All patients will receive Sargramostim (GM-CSF) 70 mcg injected subcutaneously on days 0 and −2. Patients may self administer the GM-CSF if they have been appropriately instructed on SQ injection administration. Patients will be informed of the expected reactions such as irritation at the injection site. Patients will keep a logbook noting the time and placement of the injection.
Patients will also receive 1.0 ml of emulsion of WT1 peptides with Montanide. It will be administered by a nurse (it may not be self-administered) subcutaneously at the same anatomical site as the GM-CSF.
Patients will be observed for approximately 30 minutes after vaccination.
Nivolumab will be administered intravenously as a 60-minute infusion on weeks 0, 2, 4, 6, 8, 10 and 12. Subjects may be dosed no less than 12 days between nivolumab doses and no more than 3 days after the scheduled dosing date. Dose given after the 3-day window is considered a dose delay. Treatment may be delayed for up to a maximum of 6 weeks from the previous dose.

Combination treatment of the WT1 vaccine and nivolumab is expected to increase the WT1 specific CTL population in the patient and afford increased activity against the WT1 expressing tumor, as compared to WT1 vaccination alone or nivolumab treatment alone.

### REFERENCES

1. Siegel, R., D. Naishadham, and A. Jemal, Cancer statistics, 2012. CA Cancer J Clin, 2012. 62(1): p. 10-29.
2. Hoskins, W. J., C. A. Perez, and R. C. Young, Principles and practice of gynecologic oncology. 3rd ed. 2000, Philadelphia: Lippincott Williams & Wilkins. xxi, 1268 p.
3. Barnhill, D. R., et al., The second-look surgical reassessment for epithelial ovarian carcinoma. Gynecol Oncol, 1984. 19(2): p. 148-54.
4. Rubin, S. C., et al., Recurrence after negative second-look laparotomy for ovarian cancer: analysis of risk factors. Am J Obstet Gynecol, 1988. 159(5): p. 1094-8.
5. Markman, M., et al., Second-line platinum therapy in patients with ovarian cancer previously treated with cisplatin. J Clin Oncol, 1991. 9(3): p. 389-93.
6. Zhang, H., et al., Antibodies against GD2 ganglioside can eradicate syngeneic cancer micrometastases. Cancer Res, 1998. 58(13): p. 2844-9.
7. Zhang, L., et al., Intratumoral T cells, recurrence, and survival in epithelial ovarian cancer. N Engl J Med, 2003. 348(3): p. 203-13.
8. Curiel, T. J., et al., Specific recruitment of regulatory T cells in ovarian carcinoma fosters immune privilege and predicts reduced survival. Nat Med, 2004. 10(9): p. 942-9.
9. Iasonos, A., et al., Identifying clinical improvement in consolidation single-arm phase 2 trials in patients with ovarian cancer in second or greater clinical remission. Int J Gynecol Cancer, 2012. 22(1): p. 63-9.
10. Berek, J. S., et al., Randomized, placebo-controlled study of oregovomab for consolidation of clinical remission in patients with advanced ovarian cancer. J Clin Oncol, 2004. 22(17): p. 3507-16.
11. Reinartz, S., et al., Vaccination of patients with advanced ovarian carcinoma with the anti-idiotype ACA125: immunological response and survival (phase Ib/II). Clin Cancer Res, 2004. 10(5): p. 1580-7.
12. Bookman, M. A., et al., Evaluation of monoclonal humanized anti-HER2 antibody, trastuzumab, in patients with recurrent or refractory ovarian or primary peritoneal carcinoma with overexpression of HER2: a phase II trial of the Gynecologic Oncology Group. J Clin Oncol, 2003. 21(2): p. 283-90.
13. Allavena, P., et al., Intraperitoneal recombinant gamma-interferon in patients with recurrent ascitic ovarian carcinoma: modulation of cytotoxicity and cytokine production in tumor-associated effectors and of major histocompatibility antigen expression on tumor cells. Cancer Res, 1990. 50(22): p. 7318-23.
14. Pujade-Lauraine, E., et al., Intraperitoneal recombinant interferon gamma in ovarian cancer patients with residual disease at second-look laparotomy. J Clin Oncol, 1996. 14(2): p. 343-50.
15. Recchia, F., et al., Interleukin-2 and 13-cis retinoic acid as maintenance therapy in advanced ovarian cancer. Int J Oncol, 2005. 27(4): p. 1039-46.
16. Sabbatini, P. J., et al., Immunization of ovarian cancer patients with a synthetic Lewis(y)-protein conjugate vaccine: a phase 1 trial. Int J Cancer, 2000. 87(1): p. 79-85.
17. Nicholson, S., et al., A phase I trial of idiotypic vaccination with HMFG1 in ovarian cancer. Cancer Immunol Immunother, 2004. 53(9): p. 809-16.
18. Diefenbach, C. S., et al., Safety and immunogenicity study of NY-ESO-1b peptide and montanide ISA-51 vaccination of patients with epithelial ovarian cancer in high-risk first remission. Clin Cancer Res, 2008. 14(9): p. 2740-8.
19. Keilholz, U., et al., Wilms' tumour gene 1 (WT1) in human neoplasia. Leukemia, 2005. 19(8): p. 1318-23.
20. Oji, Y., et al., Expression of the Wilms' tumor gene WT1 in solid tumors and its involvement in tumor cell growth. Jpn J Cancer Res, 1999. 90(2): p. 194-204.
21. Scharnhorst, V., et al., Internal translation initiation generates novel WT1 protein isoforms with distinct biological properties. J Biol Chem, 1999. 274(33): p. 23456-62.
22. Haber, D. A., et al., Alternative splicing and genomic structure of the Wilms tumor gene WT1. Proc Natl Acad Sci USA, 1991. 88(21): p. 9618-22.
23. Mundlos, S., et al., Nuclear localization of the protein encoded by the Wilms' tumor gene WT1 in embryonic and adult tissues. Development, 1993. 119(4): p. 1329-41.
24. Buckler, A. J., et al., Isolation, characterization, and expression of the murine Wilms' tumor gene (WT1) during kidney development. Mol Cell Biol, 1991. 11(3): p. 1707-12.
25. Fraizer, G. C., et al., Expression of the tumor suppressor gene WT1 in both human and mouse bone marrow. Blood, 1995. 86(12): p. 4704-6.
26. Al-Hussaini, M., et al., WT1 assists in distinguishing ovarian from uterine serous carcinoma and in distinguishing between serous and endometrioid ovarian carcinoma. Histopathology, 2004. 44(2): p. 109-15.
27. Pinilla-Ibarz, J., et al., Improved human T-cell responses against synthetic HLA-0201 analog peptides derived from the WT1 oncoprotein. Leukemia, 2006. 20(11): p. 2025-33.
28. May, R. J., et al., Peptide epitopes from the Wilms' tumor 1 oncoprotein stimulate CD4+ and CD8+ T cells that recognize and kill human malignant mesothelioma tumor cells. Clin Cancer Res, 2007. 13(15 Pt 1): p. 4547-55.
29. Krug, L. M., et al., WT1 peptide vaccinations induce CD4 and CD8 T cell immune responses in patients with mesothelioma and non-small cell lung cancer. Cancer Immunol Immunother, 2010. 59(10): p. 1467-79.
30. Oka, Y., et al., Induction of WT1 (Wilms' tumor gene)-specific cytotoxic T lymphocytes by WT1 peptide vaccine and the resultant cancer regression. Proc Natl Acad Sci USA, 2004. 101(38): p. 13885-90.
31. Letsch, A., et al., Effect of vaccination of leukemia patients with a MHC class I peptide of Wilms tumor gene 1 (WT1) peptide with unspecific T helper stimulation on WT1-specific IgM responses and on IgG responses. J Clin Oncol, 2008. 26: p. Abstr 3054.
32. Ohno, S., et al., Wilms' tumor 1 (WT1) peptide immunotherapy for gynecological malignancy. Anticancer Res, 2009. 29(11): p. 4779-84.
33. Schaed, S. G., et al., T-cell responses against tyrosinase 368-376(370D) peptide in HLA*A0201+ melanoma patients: randomized trial comparing incomplete Freund's adjuvant, granulocyte macrophage colony-stimulating factor, and QS-21 as immunological adjuvants. Clin Cancer Res, 2002. 8(5): p. 967-72.
34. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.
35. Faries, M. B., et al., Effect of granulocyte/macrophage colony-stimulating factor on vaccination with an allogeneic whole-cell melanoma vaccine. Clin Cancer Res, 2009. 15(22): p. 7029-35.
36. Keilholz, U., et al., A clinical and immunologic phase 2 trial of Wilms tumor gene product 1 (WT1) peptide vaccination in patients with AML and MDS. Blood, 2009. 113(26): p. 6541-8.
37. Weber, J., et al., Granulocyte-macrophage-colony-stimulating factor added to a multipeptide vaccine for resected Stage II melanoma. Cancer, 2003. 97(1): p. 186-200.
38. Keir, M. E., et al., PD-1 and its ligands in tolerance and immunity. Annu Rev Immunol, 2008. 26: p. 677-704.
39. Freeman, G. J., et al., Engagement of the PD-1 immunoinhibitory receptor by a novel B7 family member leads to negative regulation of lymphocyte activation. J Exp Med, 2000. 192(7): p. 1027-34.
40. Latchman, Y., et al., PD-L2 is a second ligand for PD-1 and inhibits T cell activation. Nat Immunol, 2001. 2(3): p. 261-8.
41. Hamanishi, J., et al., Programmed cell death 1 ligand 1 and tumor-infiltrating CD8+T lymphocytes are prognostic factors of human ovarian cancer. Proc Natl Acad Sci USA, 2007. 104(9): p. 3360-5.
42. Mu, C. Y., et al., High expression of PD-L1 in lung cancer may contribute to poor prognosis and tumor cells immune escape through suppressing tumor infiltrating dendritic cells maturation. Med Oncol, 2011. 28(3): p. 682-8.
43. Pardoll, D. M., The blockade of immune checkpoints in cancer immunotherapy. Nat Rev Cancer, 2012. 12(4): p. 252-64.
44. Nivolumab (BMS-936558) Investigator Brochure, Version 12. 2013.
45. Hwang, W. T., et al., Prognostic significance of tumor-infiltrating T cells in ovarian cancer: a meta-analysis. Gynecol Oncol, 2012. 124(2): p. 192-8.
46. Matsuzaki, J., et al., Tumor-infiltrating NY-ESO-1-specific CD8+ T cells are negatively regulated by LAG-3 and PD-1 in human ovarian cancer. Proc Natl Acad Sci USA, 2010. 107(17): p. 7875-80.
47. Brahmer, J. R., et al., Safety and activity of anti-PD-L1 antibody in patients with advanced cancer. N Engl J Med, 2012. 366(26): p. 2455-65.
48. Page, D. B., et al., Immune modulation in cancer with antibodies. Annu Rev Med, 2014. 65: p. 185-202.
49. Harrison, M. L., et al., Duration of second or greater complete clinical remission in ovarian cancer: exploring potential endpoints for clinical trials. Gynecol Oncol, 2007. 106(3): p. 469-75.
50. Juretzka, M., et al., A phase 2 trial of oral imatinib in patients with epithelial ovarian, fallopian tube, or peritoneal carcinoma in second or greater remission. Eur J Gynaecol Oncol, 2008. 29(6): p. 568-72.
51. Levine, D., et al., A phase II evaluation of goserelin and bicalutamide in patients with ovarian cancer in second or higher complete clinical disease remission. Cancer, 2007. 110(11): p. 2448-56.
52. Walter, S., et al., Multipeptide immune response to cancer vaccine IMA901 after single-dose cyclophosphamide associates with longer patient survival. Nat Med, 2012.
53. Wolchok, J. D., et al., Development of ipilimumab: a novel immunotherapeutic approach for the treatment of advanced melanoma. Ann N Y Acad Sci, 2013. 1291(1): p. 1-13.
54. Hodi, F. S., et al., Immunologic and clinical effects of antibody blockade of cytotoxic T lymphocyte-associated antigen 4 in previously vaccinated cancer patients. Proc Natl Acad Sci USA, 2008. 105(8): p. 3005-10.
55. Quezada, S. A., et al., CTLA4 blockade and GM-CSF combination immunotherapy alters the intratumor balance of effector and regulatory T cells. J Clin Invest, 2006. 116(7): p. 1935-45.
56. Duraiswamy, J., et al., Dual blockade of PD-1 and CTLA-4 combined with tumor vaccine effectively restores T-cell rejection function in tumors. Cancer Res, 2013. 73(12): p. 3591-603.
57. Wolchok, J. D., et al., Guidelines for the evaluation of immune therapy activity in solid tumors: immune-related response criteria. Clin Cancer Res, 2009. 15(23): p. 7412-20.
58. Dupont, J., et al., Wilms Tumor Gene (WT1) and p53 expression in endometrial carcinomas: a study of 130 cases using a tissue microarray. Gynecol Oncol, 2004. 94(2): p. 449-55.
59. Eisenhauer, E. A., et al., New response evaluation criteria in solid tumours: revised RECIST guideline (version 1.1). Eur J Cancer, 2009. 45(2): p. 228-47.

1. An immunotherapy composition comprising:
(a) a combination of at least seven isolated peptides consisting of:
(SEQ ID NO: 124)
YMFPNAPYL,
(SEQ ID NO: 1)
RSDELVREIFINMHQRNMTKL,
(SEQ ID NO: 2)
PGCNKRYFKLSHLQMEISRKHTG,
(SEQ ID NO: 125)
SGQAYMFPNAPYLPSCLES,
(SEQ ID NO: 21)
NLMNLGATL,
(SEQ ID NO: 26)
WNLMNLGATLKGVAA,
and
(SEQ ID NO: 205)
WNYMNLGATLKGVAA;
(b) a nucleic acid encoding the combination of at least seven isolated peptides of (a); or
(c) an immune cell comprising a nucleic acid encoding the combination of at least seven peptides of (a), and/or comprising or presenting the at least seven peptides of (a); or
(d) cytotoxic T cells (CTLs) induced by the combination of the at least seven isolated peptides of (a); or
(e) a combination of two, three, or all four from among (a), (b), (c), and (d).

2. The immunotherapy composition of claim 1, wherein the composition comprises (a) a combination of at least seven isolated peptides consisting of:
(SEQ ID NO: 124)
YMFPNAPYL,
(SEQ ID NO: 1)
RSDELVREIFINMHQRNMTKL,
(SEQ ID NO: 2)
PGCNKRYFKLSHLQMEISRKHTG,
(SEQ ID NO: 125)
SGQAYMFPNAPYLPSCLES,
(SEQ ID NO: 21)
NLMNLGATL,
(SEQ ID NO: 26)
WNLMNLGATLKGVAA,
and
(SEQ ID NO: 205)
WNYMNLGATLKGVAA;

3. The immunotherapy composition of claim 1, wherein the composition comprises (b) a nucleic acid encoding the combination of at least seven isolated peptides of:
(SEQ ID NO: 124)
YMFPNAPYL,
(SEQ ID NO: 1)
RSDELVREIFINMHQRNMTKL,
(SEQ ID NO: 2)
PGCNKRYFKLSHLQMEISRKHTG,
(SEQ ID NO: 125)
SGQAYMFPNAPYLPSCLES,
(SEQ ID NO: 21)
NLMNLGATL,
(SEQ ID NO: 26)
WNLMNLGATLKGVAA,
and
(SEQ ID NO: 205)
WNYMNLGATLKGVAA;

4. The immunotherapy composition of claim 1, wherein the nucleic acid of (b) is in, or otherwise associated with, a viral or non-viral vector.

5. The immunotherapy composition of claim 1, wherein the composition comprises (c) an immune cell comprising a nucleic acid encoding the combination of at least seven peptides of (a), and/or comprising or presenting the at least seven peptides of (a):
(SEQ ID NO: 124)
YMFPNAPYL,
(SEQ ID NO: 1)
RSDELVREIFINMHQRNMTKL,
(SEQ ID NO: 2)
PGCNKRYFKLSHLQMEISRKHTG,
(SEQ ID NO: 125)
SGQAYMFPNAPYLPSCLES,
(SEQ ID NO: 21)
NLMNLGATL,
(SEQ ID NO: 26)
WNLMNLGATLKGVAA,
and
(SEQ ID NO: 205)
WNYMNLGATLKGVAA;

6. The immunotherapy composition of claim 1, wherein the composition comprises (d) CTLs induced by the combination of at least seven isolated peptides of (a), and wherein the CTLs are produced in vitro, or produced ex vivo, or produced in vivo and obtained from a donor.

7. The immunotherapy composition of claim 1, wherein the composition comprises (e) a combination of two, three, or all four from among (a), (b), (c), and (d).

8. (canceled)

9. The immunotherapy composition of claim 1, wherein the combination of peptides consists of only the seven isolated peptides.

10. The immunotherapy composition of claim 1, further comprising an antigen presenting cell, carrier, vehicle, diluent, or adjuvant.

11. The immunotherapy composition of claim 10, wherein the composition further comprises the adjuvant, and the adjuvant is QS21, Freund's incomplete adjuvant, aluminum phosphate, aluminum hydroxide, BCG, alum, a growth factor, a cytokine, a chemokine, an interleukin, Montanide ISA 51, or GM-CSF.

12. The immunotherapy composition of claim 1, wherein the seven peptides are present in equal amounts.

13. The immunotherapy composition of claim 1, wherein the seven peptides are not present in equal amounts.

14. The immunotherapy composition of claim 1, wherein the combination of peptides induce a class I response and a class II response.

15. The immunotherapy composition of claim 1, wherein the combination of peptides induces a CD4+ response, a CD8+ response, or the combination thereof.

16. The immunotherapy composition of claim 1, wherein T cells are formed in subjects having HLA*A02, HLA*A03, HLA*B07, HLA*A24, or any combination of two or more of the foregoing.

17. The immunotherapy composition of claim 1, wherein the ratio of seven peptides comprises 0.1 to 10 parts YMFPNAPYL (SEQ ID NO: 124), 0.1 to 10 parts RSDELVRHHNMHQRNMTKL (SEQ ID NO: 1), 0.1 to 10 parts PGCNKRYFKLSHLQMHSRKHTG (SEQ ID NO: 2), 0.1 to 10 parts SGQAYMFPNAPYLPSCLES (SEQ ID NO: 125), 0.1 to 10 parts NLMNLGATL (SEQ ID NO: 103), 0.1 to 10 parts WNLMNLGATLKGVAA (SEQ ID NO: 26), and 0.1 to 10 parts WNYMNLGATLKGVAA (SEQ ID NO: 205).

18. The immunotherapy composition of claim 1, wherein the ratio of seven peptides to one another is proportionate to the relative strength of the seven peptides' HLA binding scores from one or more predictive algorithms.

19. A method for treating, reducing the incidence of, or inducing an immune response against a WT1-expressing cancer, the method comprising administering to a subject in need thereof one or more of the following:
(a) a combination of at least seven isolated peptides consisting of:
(SEQ ID NO: 124)
YMFPNAPYL,
(SEQ ID NO: 1)
RSDELVREIFINMHQRNMTKL,
(SEQ ID NO: 2)
PGCNKRYFKLSHLQMEISRKHTG,
(SEQ ID NO: 125)
SGQAYMFPNAPYLPSCLES,
(SEQ ID NO: 21)
NLMNLGATL,
(SEQ ID NO: 26)
WNLMNLGATLKGVAA,
and
(SEQ ID NO: 205)
WNYMNLGATLKGVAA;
(b) a nucleic acid encoding the combination of at least seven isolated peptides of (a); or
(c) an immune cell comprising a nucleic acid encoding the combination of at least seven peptides of (a), and/or comprising or presenting the at least seven peptides of (a); or
(d) cytotoxic T cells (CTLs) against the WT1-expressing cancer, wherein the CTLs are induced by the combination of the at least seven isolated peptides of (a); or
(e) a combination of two, three, or all four from among (a), (b), (c), and (d).

20-31. (canceled)

32. The method of claim 19, further comprising administering at least one checkpoint inhibitor to the subject.

33-34. (canceled)

35. A method for inducing the formation and proliferation of T cells specific for a WT1-expressing cancer in a subject, the method comprising administering to a subject one or more of the following WT1 delivery agents:
(a) a combination of at least seven isolated peptides consisting of:
(SEQ ID NO: 124)
YMFPNAPYL,
(SEQ ID NO: 1)
RSDELVREIFINMHQRNMTKL,
(SEQ ID NO: 2)
PGCNKRYFKLSHLQMEISRKHTG,
(SEQ ID NO: 125)
SGQAYMFPNAPYLPSCLES,
(SEQ ID NO: 21)
NLMNLGATL,
(SEQ ID NO: 26)
WNLMNLGATLKGVAA,
and
(SEQ ID NO: 205)
WNYMNLGATLKGVAA;
(b) a nucleic acid encoding the combination of at least seven isolated peptides of (a);
(c) an immune cell comprising a nucleic acid encoding the combination of at least seven peptides of (a), and/or comprising or presenting the at least seven peptides of (a); or
(d) a combination of two or three from among (a), (b), and (c).

36-49. (canceled)


SEQUENCE LISTING (205 sequences)

SEQ ID NO: 1 - protein, 19 aa, Homo sapiens
RSDELVRHHNMHQRNMTKL

SEQ ID NO: 2 - protein, 22 aa, Homo sapiens
PGCNKRYFKLSHLQMHSRKHTG

SEQ ID NO: 3 - protein, 15 aa, Homo sapiens
LVRHHNMHQRNMTKL

SEQ ID NO: 4 - protein, 15 aa, Homo sapiens
NKRYFKLSHLQMHSR

SEQ ID NO: 5 - protein, 19 aa, Homo sapiens
SGQARMFPNAPYLPSCLES

SEQ ID NO: 6 - protein, 15 aa, Homo sapiens
QARMFPNAPYLPSCL

SEQ ID NO: 7 - protein, 9 aa, Homo sapiens
RMFPNAPYL

SEQ ID NO: 8 - protein, 9 aa, Homo sapiens
SLGEQQYSV

SEQ ID NO: 9 - protein, 9 aa, Homo sapiens
ALLPAVPSL

SEQ ID NO: 10 - protein, 9 aa, Homo sapiens
NLGATLKGV

SEQ ID NO: 11 - protein, 9 aa, Homo sapiens
DLNALLPAV

SEQ ID NO: 12 - protein, 9 aa, Homo sapiens
GVFRGIQDV

SEQ ID NO: 13 - protein, 9 aa, Homo sapiens
KRYFKLSHL

SEQ ID NO: 14 - protein, 9 aa, Homo sapiens
ALLLRTPYS

SEQ ID NO: 15 - protein, 9 aa, Homo sapiens
CMTWMQMNL

SEQ ID NO: 16 - protein, 9 aa, Homo sapiens
NMHQRNMTK

SEQ ID NO: 17 - protein, 9 aa, Homo sapiens
QMNLGATLK

SEQ ID NO: 18 - protein, 10 aa, Homo sapiens
FMCAYPGCNK

SEQ ID NO: 19 - protein, 10 aa, Homo sapiens
KLSHLQMHSR

SEQ ID NO: 20 - protein, 9 aa, Homo sapiens
NQMNLGATL

SEQ ID NO: 21 - protein, 9 aa, Artificial Sequence
  Mutated Homo sapiens
NLMNLGATL

SEQ ID NO: 22 - protein, 9 aa, Artificial Sequence
  Mutated Homo sapiens
NYMNLGATL

SEQ ID NO: 23 - protein, 15 aa, Homo sapiens
CMTWNQMNLGATLKG

SEQ ID NO: 24 - protein, 15 aa, Artificial Sequence
  Mutated Homo sapiens
CMTWNLMNLGATLKG

SEQ ID NO: 25 - protein, 15 aa, Homo sapiens
WNQMNLGATLKGVAA

SEQ ID NO: 26 - protein, 15 aa, Artificial Sequence
  Mutated Homo sapiens
WNLMNLGATLKGVAA

SEQ ID NO: 27 - protein, 15 aa, Homo sapiens
MTWNQMNLGATLKGV

SEQ ID NO: 28 - protein, 15 aa, Homo sapiens
TWNQMNLGATLKGVA

SEQ ID NO: 29 - protein, 15 aa, Homo sapiens
CMTWNLMNLGATLKG

SEQ ID NO: 30 - protein, 15 aa, Artificial Sequence
  Mutated Homo sapiens
MTWNLMNLGATLKGV

SEQ ID NO: 31 - protein, 15 aa, Artificial Sequence
  Mutated Homo sapiens
TWNLMNLGATLKGVA

SEQ ID NO: 32 - protein, 15 aa, Artificial Sequence
  Mutated Homo sapiens
WNLMNLGATLKGVAA

SEQ ID NO: 33 - protein, 15 aa, Homo sapiens
MTWNYMNLGATLKGV

SEQ ID NO: 34 - protein, 15 aa, Artificial Sequence
  Mutated Homo sapiens
TWNYMNLGATLKGVA

SEQ ID NO: 35 - protein, 17 aa, Homo sapiens
CMTWNQMNLGATLKGVA

SEQ ID NO: 36 - protein, 9 aa, Homo sapiens
WNQMNLGAT

SEQ ID NO: 37 - protein, 9 aa, Homo sapiens
TWNQMNLGA

SEQ ID NO: 38 - protein, 9 aa, Homo sapiens
MTWNQMNLG

SEQ ID NO: 39 - protein, 17 aa, Artificial Sequence
  Mutated Homo sapiens
CMTWNLMNLGATLKGVA

SEQ ID NO: 40 - protein, 9 aa, Artificial Sequence
  Mutated Homo sapiens
WNLMNLGAT

SEQ ID NO: 41 - protein, 9 aa, Artificial Sequence
  Mutated Homo sapiens
MNLGATLKG

SEQ ID NO: 42 - protein, 9 aa, Homo sapiens
MTWNQMNLG

SEQ ID NO: 43 - protein, 17 aa, Artificial Sequence
  Mutated Homo sapiens
CMTWNYMNLGATLKGVA

SEQ ID NO: 44 - protein, 9 aa, Homo sapiens
MNLGATLKG

SEQ ID NO: 45 - protein, 9 aa, Homo sapiens
MTWNQMNLG

SEQ ID NO: 46 - protein, 9 aa, Homo sapiens
GALRNPTAC

SEQ ID NO: 47 - protein, 9 aa, Homo sapiens
GYLRNPTAC

SEQ ID NO: 48 - protein, 9 aa, Homo sapiens
GALRNPTAL

SEQ ID NO: 49 - protein, 9 aa, Homo sapiens
YALRNPTAC

SEQ ID NO: 50 - protein, 9 aa, Homo sapiens
GLLRNPTAC

SEQ ID NO: 51 - protein, 9 aa, Homo sapiens
RQRPHPGAL

SEQ ID NO: 52 - protein, 9 aa, Homo sapiens
RYRPHPGAL

SEQ ID NO: 53 - protein, 9 aa, Homo sapiens
YQRPHPGAL

SEQ ID NO: 54 - protein, 9 aa, Homo sapiens
RLRPHPGAL

SEQ ID NO: 55 - protein, 9 aa, Homo sapiens
RIRPHPGAL

SEQ ID NO: 56 - protein, 9 aa, Homo sapiens
GALRNPTAC

SEQ ID NO: 57 - protein, 9 aa, Homo sapiens
GALRNPTAL

SEQ ID NO: 58 - protein, 9 aa, Homo sapiens
RQRPHPGAL

SEQ ID NO: 59 - protein, 9 aa, Homo sapiens
RLRPHPGAL

SEQ ID NO: 60 - protein, 9 aa, Homo sapiens
RIRPHPGAL

SEQ ID NO: 61 - protein, 15 aa, Homo sapiens
QFPNHSFKHEDPMGQ

SEQ ID NO: 62 - protein, 15 aa, Homo sapiens
QFPNHSFKHEDPMGQ

SEQ ID NO: 63 - protein, 9 aa, Homo sapiens
HSFKHEDPM

SEQ ID NO: 64 - protein, 9 aa, Homo sapiens
HSFKHEDPY

SEQ ID NO: 65 - protein, 9 aa, Homo sapiens
HSFKHEDPK

SEQ ID NO: 66 - protein, 15 aa, Homo sapiens
KRPFMCAYPGCYKRY

SEQ ID NO: 67 - protein, 15 aa, Homo sapiens
SEKRPFMCAYPGCNK

SEQ ID NO: 68 - protein, 13 aa, Homo sapiens
KRPFMCAYPGCNK

SEQ ID NO: 69 - protein, 9 aa, Homo sapiens
FMCAYPGCN

SEQ ID NO: 70 - protein, 9 aa, Homo sapiens
FMCAYPGCY

SEQ ID NO: 71 - protein, 9 aa, Homo sapiens
FMCAYPGCK

SEQ ID NO: 72 - protein, 9 aa, Homo sapiens
RQRPHPGAL

SEQ ID NO: 73 - protein, 9 aa, Homo sapiens
GALRNPTAC

SEQ ID NO: 74 - protein, 9 aa, Homo sapiens
PLPHFPPSL

SEQ ID NO: 75 - protein, 9 aa, Homo sapiens
HFPPSLPPT

SEQ ID NO: 76 - protein, 9 aa, Homo sapiens
THSPTHPPR

SEQ ID NO: 77 - protein, 9 aa, Homo sapiens
AILDFLLLQ

SEQ ID NO: 78 - protein, 9 aa, Homo sapiens
PGCLQQPEQ

SEQ ID NO: 79 - protein, 11 aa, Homo sapiens
PGCLQQPEQQG

SEQ ID NO: 80 - protein, 9 aa, Homo sapiens
KLGAAEASA

SEQ ID NO: 81 - protein, 9 aa, Homo sapiens
ASGSEPQQM

SEQ ID NO: 82 - protein, 10 aa, Homo sapiens
RDLNALLPAV

SEQ ID NO: 83 - protein, 10 aa, Homo sapiens
GGCALPVSGA

SEQ ID NO: 84 - protein, 9 aa, Homo sapiens
GAAQWAPVL

SEQ ID NO: 85 - protein, 9 aa, Homo sapiens
LDFAPPGAS

SEQ ID NO: 86 - protein, 11 aa, Homo sapiens
LDFAPPGASAY

SEQ ID NO: 87 - protein, 9 aa, Homo sapiens
SAYGSLGGP

SEQ ID NO: 88 - protein, 9 aa, Homo sapiens
PAPPPPPPP

SEQ ID NO: 89 - protein, 9 aa, Homo sapiens
ACRYGPFGP

SEQ ID NO: 90 - protein, 9 aa, Homo sapiens
SGQARMFPN

SEQ ID NO: 91 - protein, 9 aa, Homo sapiens
RMFPNAPYL

SEQ ID NO: 92 - protein, 9 aa, Homo sapiens
PSCLESQPA

SEQ ID NO: 93 - protein, 9 aa, Homo sapiens
NQGYSTVTF

SEQ ID NO: 94 - protein, 9 aa, Homo sapiens
HHAAQFPNH

SEQ ID NO: 95 - protein, 9 aa, Homo sapiens
HSFKHEDPM

SEQ ID NO: 96 - protein, 9 aa, Homo sapiens
CHTPTDSCT

SEQ ID NO: 97 - protein, 9 aa, Homo sapiens
CTGSQALLL

SEQ ID NO: 98 - protein, 9 aa, Homo sapiens
TDSCTGSQA

SEQ ID NO: 99 - protein, 9 aa, Homo sapiens
RTPYSSDNL

SEQ ID NO: 100 - protein, 10 aa, Homo sapiens
NLYQMTSQLE

SEQ ID NO: 101 - protein, 9 aa, Homo sapiens
WNQMNLGAT

SEQ ID NO: 102 - protein, 9 aa, Artificial Sequence
  Mutated Homo sapiens
NQMNLGATL

SEQ ID NO: 103 - protein, 11 aa, Homo sapiens
WNQMNLGATLK

SEQ ID NO: 104 - protein, 15 aa, Homo sapiens
CMTWNQMNLGATLKG

SEQ ID NO: 105 - protein, 9 aa, Homo sapiens
NLGATLKGV

SEQ ID NO: 106 - protein, 10 aa, Homo sapiens
LGATLKGVAA

SEQ ID NO: 107 - protein, 8 aa, Homo sapiens
TLGVAAGS

SEQ ID NO: 108 - protein, 9 aa, Homo sapiens
GYESDNHTT

SEQ ID NO: 109 - protein, 10 aa, Homo sapiens
FMCAYPGCNK

SEQ ID NO: 110 - protein, 11 aa, Homo sapiens
KRPFMCAYPGC

SEQ ID NO: 111 - protein, 9 aa, Homo sapiens
RKFSRSDHL

SEQ ID NO: 112 - protein, 10 aa, Homo sapiens
LKTHTTRTHT

SEQ ID NO: 113 - protein, 10 aa, Homo sapiens
NMHQRNHTKL

SEQ ID NO: 114 - protein, 9 aa, Homo sapiens
LLAAILDFL

SEQ ID NO: 115 - protein, 10 aa, Homo sapiens
CLQQPEQQGV

SEQ ID NO: 116 - protein, 9 aa, Homo sapiens
DLNALLPAV

SEQ ID NO: 117 - protein, 9 aa, Homo sapiens
ALLPAVPSL

SEQ ID NO: 118 - protein, 9 aa, Homo sapiens
VLDFAPPGA

SEQ ID NO: 119 - protein, 9 aa, Homo sapiens
CMTWNQMNL

SEQ ID NO: 120 - protein, 10 aa, Homo sapiens
QARMFPNAPY

SEQ ID NO: 121 - protein, 10 aa, Homo sapiens
ALRNPTACPL

SEQ ID NO: 122 - protein, 9 aa, Homo sapiens
YPGCNKRYF

SEQ ID NO: 123 - protein, 15 aa, Homo sapiens
APVLDFAPPGASAYG

SEQ ID NO: 124 - protein, 9 aa, Artificial Sequence
  Mutated Homo sapiens
YMFPNAPYL

SEQ ID NO: 125 - protein, 19 aa, Artificial Sequence
  Mutated Homo sapiens
SGQAYMFPNAPYLPSCLES

SEQ ID NO: 126 - protein, 15 aa, Artificial Sequence
  Mutated Homo sapiens
QAYMFPNAPYLPSCL

SEQ ID NO: 127 - protein, 9 aa, Homo sapiens
YLGEQQYSV

SEQ ID NO: 128 - protein, 9 aa, Homo sapiens
YLLPAVPSL

SEQ ID NO: 129 - protein, 9 aa, Homo sapiens
YLGATLKGV

SEQ ID NO: 130 - protein, 9 aa, Homo sapiens
YLNALLPAV

SEQ ID NO: 131 - protein, 9 aa, Homo sapiens
GLRRGIQDV

SEQ ID NO: 132 - protein, 9 aa, Homo sapiens
KLYFKLSHL

SEQ ID NO: 133 - protein, 9 aa, Homo sapiens
ALLLRTPYV

SEQ ID NO: 134 - protein, 9 aa, Homo sapiens
YMTWNQMNL

SEQ ID NO: 135 - protein, 9 aa, Homo sapiens
NMYQRNMTK

SEQ ID NO: 136 - protein, 9 aa, Homo sapiens
NMHQRVMTK

SEQ ID NO: 137 - protein, 9 aa, Homo sapiens
NMYQRVMTK

SEQ ID NO: 138 - protein, 9 aa, Homo sapiens
QMYLGATLK

SEQ ID NO: 139 - protein, 9 aa, Homo sapiens
QMNLGVTLK

SEQ ID NO: 140 - protein, 9 aa, Homo sapiens
QMYLGVTLK

SEQ ID NO: 141 - protein, 10 aa, Homo sapiens
FMYAYPGCNK

SEQ ID NO: 142 - protein, 10 aa, Homo sapiens
FMCAYPFCNK

SEQ ID NO: 143 - protein, 10 aa, Homo sapiens
FMYAYPFCNK

SEQ ID NO: 144 - protein, 10 aa, Homo sapiens
KLYHLQMHSR

SEQ ID NO: 145 - protein, 10 aa, Homo sapiens
KLSHLQMHSK

SEQ ID NO: 146 - protein, 10 aa, Homo sapiens
KLYHLQMHSK

SEQ ID NO: 147 - protein, 9 aa, Homo sapiens
NQMNLGATL

SEQ ID NO: 148 - skipped

SEQ ID NO: 149 - protein, 9 aa, Homo sapiens
NYMNLGATL

SEQ ID NO: 150 - protein, 15 aa, Homo sapiens
CMTWNQMNLGATLKG

SEQ ID NO: 151 - protein, 15 aa, Homo sapiens
CMTWNLMNLGATLKG

SEQ ID NO: 152 - protein, 15 aa, Homo sapiens
WNQMNLGATLKGVAA

SEQ ID NO: 153 - skipped

SEQ ID NO: 154 - protein, 15 aa, Homo sapiens
MTWNQMNLGATLKGV

SEQ ID NO: 155 - protein, 15 aa, Homo sapiens
TWNQMNLGATLKGVA

SEQ ID NO: 156 - protein, 15 aa, Homo sapiens
CMTWNLMNLGATLKG

SEQ ID NO: 157 - protein, 15 aa, Homo sapiens
MTWNLMNLGATLKGV

SEQ ID NO: 158 - protein, 15 aa, Homo sapiens
TWNLMNLGATLKGVA

SEQ ID NO: 159 - protein, 15 aa, Homo sapiens
WNLMNLGATLKGVAA

SEQ ID NO: 160 - protein, 15 aa, Homo sapiens
MTWNYMNLGATLKGV

SEQ ID NO: 161 - protein, 15 aa, Homo sapiens
TWNYMNLGATLKGVA

SEQ ID NO: 162 - protein, 17 aa, Homo sapiens
CMTWNQMNLGATLKGVA

SEQ ID NO: 163 - protein, 9 aa, Homo sapiens
WNQMNLGAT

SEQ ID NO: 164 - protein, 9 aa, Homo sapiens
TWNQMNLGA

SEQ ID NO: 165 - protein, 9 aa, Homo sapiens
MTWNQMNLG

SEQ ID NO: 166 - protein, 17 aa, Homo sapiens
CMTWNLMNLGATLKGVA

SEQ ID NO: 167 - protein, 9 aa, Homo sapiens
WNLMNLGAT

SEQ ID NO: 168 - protein, 9 aa, Homo sapiens
MNLGATLKG

SEQ ID NO: 169 - protein, 9 aa, Homo sapiens
MTWNQMNLG

SEQ ID NO: 170 - protein, 17 aa, Homo sapiens
CMTWNYMNLGATLKGVA

SEQ ID NO: 171 - protein, 9 aa, Homo sapiens
MNLGATLKG

SEQ ID NO: 172 - protein, 9 aa, Homo sapiens
MTWNQMNLG

SEQ ID NO: 173 - protein, 9 aa, Homo sapiens
GALRNPTAC

SEQ ID NO: 174 - protein, 9 aa, Homo sapiens
GYLRNPTAC

SEQ ID NO: 175 - protein, 9 aa, Homo sapiens
GALRNPTAL

SEQ ID NO: 176 - protein, 9 aa, Homo sapiens
YALRNPTAC

SEQ ID NO: 177 - protein, 9 aa, Homo sapiens
GLLRNPTAC

SEQ ID NO: 178 - protein, 9 aa, Homo sapiens
RQRPHPGAL

SEQ ID NO: 179 - protein, 9 aa, Homo sapiens
RYRPHPGAL

SEQ ID NO: 180 - protein, 9 aa, Homo sapiens
YQRPHPGAL

SEQ ID NO: 181 - protein, 9 aa, Homo sapiens
RLRPHPGAL

SEQ ID NO: 182 - protein, 9 aa, Homo sapiens
RIRPHPGAL

SEQ ID NO: 183 - protein, 9 aa, Homo sapiens
GALRNPTAC

SEQ ID NO: 184 - protein, 9 aa, Homo sapiens
GALRNPTAL

SEQ ID NO: 185 - protein, 9 aa, Homo sapiens
RQRPHPGAL

SEQ ID NO: 186 - protein, 9 aa, Homo sapiens
RLRPHPGAL

SEQ ID NO: 187 - protein, 9 aa, Homo sapiens
RIRPHPGAL

SEQ ID NO: 188 - protein, 15 aa, Homo sapiens
QFPNHSFKHEDPMGQ

SEQ ID NO: 189 - protein, 15 aa, Homo sapiens
QFPNHSFKHEDPMGQ

SEQ ID NO: 190 - protein, 9 aa, Homo sapiens
HSFKHEDPM

SEQ ID NO: 191 - protein, 9 aa, Homo sapiens
HSFKHEDPY

SEQ ID NO: 192 - protein, 9 aa, Homo sapiens
HSFKHEDPK

SEQ ID NO: 193 - protein, 15 aa, Homo sapiens
KRPFMCAYPGCYKRY

SEQ ID NO: 194 - protein, 15 aa, Homo sapiens
SEKRPFMCAYPGCNK

SEQ ID NO: 195 - protein, 13 aa, Homo sapiens
KRPFMCAYPGCNK

SEQ ID NO: 196 - protein, 9 aa, Homo sapiens
FMCAYPGCN

SEQ ID NO: 197 - protein, 9 aa, Homo sapiens
FMCAYPGCY

SEQ ID NO: 198 - protein, 9 aa, Homo sapiens
FMCAYPGCK

SEQ ID NO: 199 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 200 - protein, 453 aa, Homo sapiens
AAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 201 - protein, 514 aa, Homo sapiens
MQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 202 - protein, 168 aa, Homo sapiens
MGHHHHHHHHHHSSGHIEGRHMRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 203 - protein, 18 aa, Homo sapiens
GATLKGVAAGSSSSVKWT

SEQ ID NO: 204 - protein, 15 aa, Homo sapiens
LKGVAAGSSSSVKWT

SEQ ID NO: 205 - protein, 15 aa, Artificial Sequence
  Mutated Homo sapiens
WNYMNLGATLKGVAA